(19) United States
(12) Patent Application Publication
(10) Pub. No.: US 2023/0026840 A1
(43) Pub. Date: Jan. 26, 2023

(54) COMPOUND USED AS EGFR KINASE INHIBITOR AND USE THEREOF

(71) Applicant: TYK MEDICINES, INC., Huzhou (CN)

(72) Inventors: Yusheng WU, Huzhou (CN); Jun LI, Huzhou (CN); Chengshan NIU, Huzhou (CN); Maolin ZHENG, Huzhou (CN); Apeng LIANG, Huzhou (CN)

(73) Assignee: TYK MEDICINES, INC., Huzhou (CN)

(21) Appl. No.: 17/764,903

(22) PCT Filed: Nov. 23, 2020
(86) PCT No.: PCT/CN2020/130948

§ 371 Date: Mar. 29, 2022

(30) Foreign Application Priority Data
Nov. 21, 2019 (CN) 201911150532.2

(51) Int. Cl. C07F 9/6558; C07F 9/6561; A61P 35/00

(52) U.S. Cl. CPC C07F 9/65583; C07F 9/6561; A61P 35/00

(57) ABSTRACT

The present invention relates to a compound used as an EGFR kinase inhibitor and the use thereof. The compound has a structure as shown in formula I, and can be used to adjust the kinase activity or treat related diseases, especially non-small cell lung cancer.

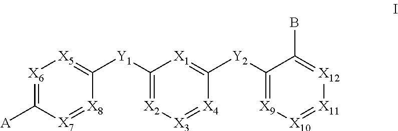

### TECHNICAL FIELD

The invention relates to the technical field of medicine, in particular to a compound used as EGFR kinase inhibitor and preparation thereof, for regulating EGFR kinase activity or treating related diseases, especially non-small cell lung cancer.

### BACKGROUND OF THE INVENTION

Tumor is one of the most important problems that endanger human health, and lung cancer is one of the most threatening malignant tumors to people's health and life. Lung cancer is mainly divided into small cell lung cancer (SCLC) and non-small cell lung cancer (NSCLC), of which about 80% are NSCLC. The most common mutation and the mutation having targeted drugs in non-small cell lung cancer is the “epidermal growth factor receptor” (EGFR) mutation. Therefore, the use of epidermal growth factor receptor EGFR inhibitors (EGFR-TKI targeted drugs) is one of the biggest research hotspots in the treatment of lung cancer. EGFR (Epidermal Growth Factor Receptor) is a receptor for epithelial growth factor (EGF) cell proliferation and signal transduction. Studies have shown that there are high or abnormal expressions of EGFR in many solid tumors. EGFR is related to the inhibition of tumor cell proliferation, angiogenesis, tumor invasion, metastasis and apoptosis.

Currently, the EGFR-TKIs that have been marketed include the first-generation inhibitors Iressa, Tarceva, and Conmana, the second-generation inhibitors Afatinib and Dacomitinib, and the third-generation inhibitor Osimertinib, making EGFR-positive non-small cells lung cancer patient benefit from EGFR-TKI treatment. However, in the course of treatment, the resistance of tumors to drug-resistant mutations is an inevitable problem. After first-generation and second-generation EGFR-TKI treatments, approximately 60% of patients will develop T790M resistance mutations, causing the first and second-generation drugs to lose their therapeutic effects. As the third-generation EGFR-TKI, Osimertinib has very good inhibitory activity on T790M, which can bring better treatment effects and survival benefits to patients. However, after Osimertinib is used for a period of time, the tumor will develop drug-resistant mutations again, and about 20-30% of patients will develop C797S drug-resistant mutations (nature medicine, 21, 560-562, 2016). With the approval of Osimertinib as the first-line treatment for EGFR-positive non-small cell lung cancer, more drug-resistant patients with C797S mutations will appear. At present, there is no targeted drug that can treat this drug-resistant mutation on the market.

An EGFR allosteric inhibitor EAI045 (nature, 534, 129-132, 2016) was reported in Nature in 2016, and its combination with cetuximab can inhibit tumors with L858/T790M/C797S mutations. In 2017, “nature communications, 8, 14768, 2017” reported that the combination of brigatinib and cetuximab in the PC9 (EGFR-C797S/T790M/del19) mouse pharmacodynamic model showed better pharmacodynamic efficacy. However, until now, there have been no clinical reports of brigatinib in this field.

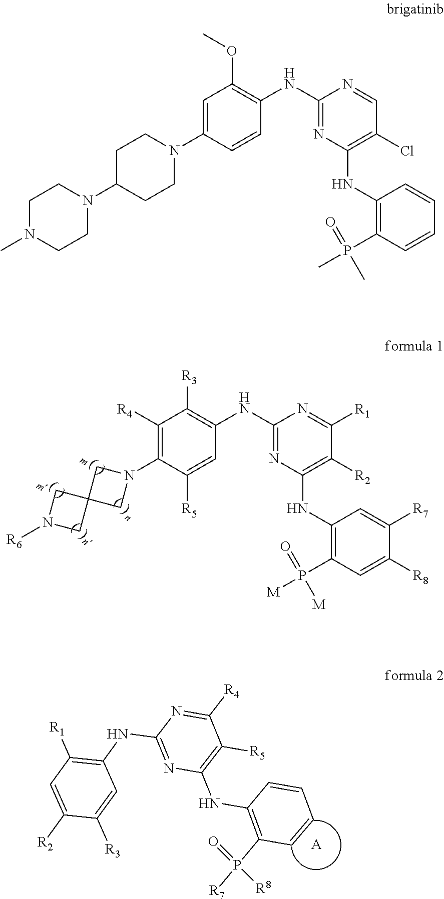

WO2018108064A1 reported a spirocyclic aryl phosphorus oxide compound (general formula shown in formula 1) as a fourth-generation EGFR inhibit or. According to the activity data in the patent, most of the compounds inhibit C797S/T790M at 100 nM and above. WO2019015655A1 discloses aryl phosphorus-oxygen compounds (general formula shown in formula 2) as EGFR kinase inhibitors. The compounds in this patent have good enzymatic activity against EGFR(deI19/T790M/C797S) and while the cell activity is only in the tens of nanomoles for individual compounds, and most of them are in the hundreds to thousands of nanomoles, and the R₂ in the general formula is selected from H, F, Cl, Br, CN, OH, NH₂, NO₂,

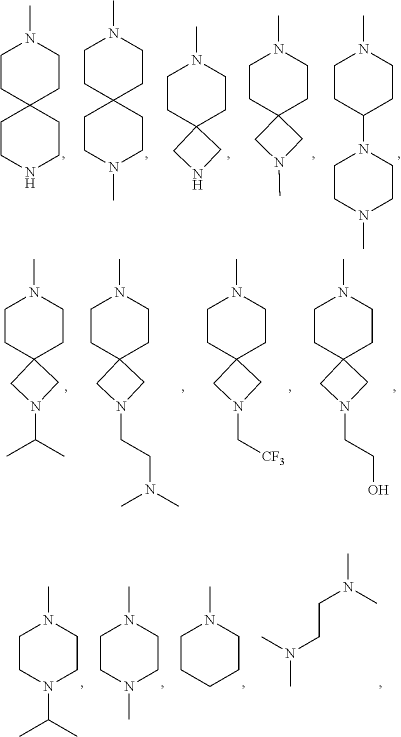

ethylamine, methylamine and dimethylamine. The examples of this patent are basically spirocyclic amine derivatives and

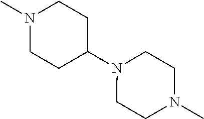

derivatives. WO2019007293A1 reports an aryl phosphorus-oxygen compound with a similar structure, but it is a compound used for ALK inhibitors. Recently, CN110305161A also reported an aryl phosphorus oxide compound similar to the patent WO2019015655A1. Therefore, targeting the C797S mutation, overcoming the resistance of Osimertinib, and providing patients with safer and more effective fourth-generation EGFR inhibitors have very important research significance.

### SUMMARY OF THE INVENTION

In the first aspect of the invention, it provides a compound of formula I or a pharmaceutically acceptable salt, solvate or prodrug thereof,

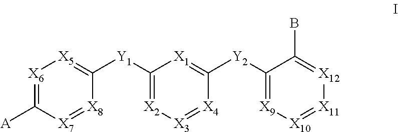

wherein,

X₁ is selected from N or CR₁;

X₂ is selected from N or CR₂;

X₃ is selected from N or CR₃;

X₄ is selected from N or CR₄;

X₅ is selected from N or CR₅;

X₆ is selected from N or CR₆;

X₇ is selected from N or CR₇;

X₈ is selected from N or CR₈;

X₉ is selected from N or CR₉;

X₁₀ is selected from N or CR₁₀;

X₁₁ is selected from N or CR₁₁;

X₁₂ is selected from N or CR₁₂;

Y₁ and Y₂ are each independently selected from the divalent group consisting of —O—, —S—, —S(O)—, —S(O)₂—,

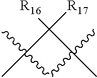

### and —NR₁₈—;

A is selected from the group consisting of

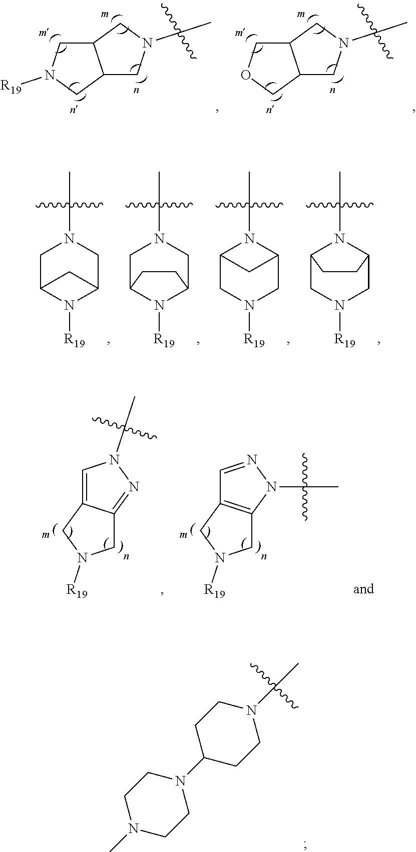

or A and X₇ or X₆ form a substituted 5-7 membered ring;

B is selected from the group consisting of

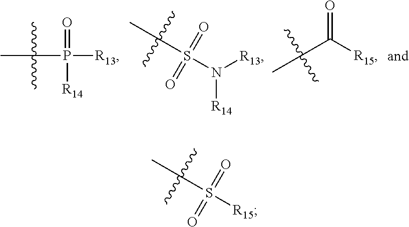

R₁, R₂, R₃, R₄, R₅, R₆, R₇, R₈, R₉, R₁₀, R₁₁ and R₁₂ are each independently selected from the substituted or unsubstituted group consisting of H, halogen, CN, NH₂, ester, carbamido, carbamate, amido, C₁₋₆ alkyl, C₁₋₆ alkoxy, C₃₋₆ cycloalkyl, C₃₋₆ cycloalkoxy, sufonamido, amino, 3-10 membered heterocyclyl, C₆-C₁₀ aryl, and 5-14 membered heteroaryl;

or R₃ and X₂ or X₄ form a substituted or unsubstituted 5-7 membered ring containing 0-3 heteroatoms selected from O, S, and N;

or R₁₁ and X₁₀ or X₁₂ form a substituted or unsubstituted 5-7 membered ring containing 0-3 heteroatoms selected from O, S, and N;

or R₁₀ and X₉ or X₁₁ form a substituted or unsubstituted 5-7 membered ring containing 0-3 heteroatoms selected from O, S, and N;

or R₅ and X₆ form a substituted or unsubstituted 5-7 membered ring containing 0-3 heteroatoms selected from O, S, and N;

or R₇ and X₈ form a substituted or unsubstituted 5-7 membered ring containing 0-3 heteroatoms selected from O, S, and N;

R₁₃, R₁₄ and R₁₅ are each independently selected from the substituted or unsubstituted group consisting of H, C₁₋₆ alkyl, C₁₋₆ alkoxy, C₃₋₈ cycloalkyl, C₃₋₈ cycloalkoxy, C₆-C₁₀ aryl, and 5-14 membered heteroaryl;

or R₁₃ and R₁₄ together with the P or N atoms to which they are attached form a substituted or unsubstituted 4-8 membered heterocyclyl;

R₁₆, R₁₇ and R₁₈ are each independently selected from the substituted or unsubstituted group consisting of H, halogen, C₁₋₆ alkyl, C₁₋₆ alkoxy, C₃₋₈ cycloalkyl, C₃₋₈ cycloalkoxy, C₆-C₁₀ aryl, and 5-14 membered heteroaryl;

or R₁₆ and R₁₇ together with the C atoms to which they are attached form a substituted or unsubstituted C₄₋₈ cycloalkyl or 4-8 membered heterocyclyl;

R₁₉ is selected from the substituted or unsubstituted group consisting of H, C₁₋₆ alkyl, C₁₋₆ alkoxy, C₃₋₈ cycloalkyl, C₃₋₈ cycloalkoxy, C₆-C₁₀ aryl, 5-14 membered heteroaryl, C₁₋₆ alkoxycarbonyl, C₁₋₆ alkylcarbonyl, and C₁₋₆ alkyl-S(═O)₂—;

m, n, m′ and n′ are each independently 0, 1, 2, or 3;

with the proviso that

when A is

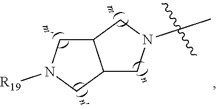

X₁ and X₂ are not N at the same time;

or X₅ is CR₅, and R₅ is selected from the substituted or unsubstituted group consisting of halogen, CN, NH₂, ester, carbamido, carbamate, amido, C₁₋₆ alkyl, C₁₋₆ alkoxy, C₃₋₆ cycloalkyl, C₃₋₆ cycloalkoxy, C₆-C₁₀ aryl, and 5-14 membered heteroaryl;

or X₆ is CR₆, and R₆ is selected from the substituted or unsubstituted group consisting of halogen, CN, NH₂, ester, carbamido, carbamate, amido, C₁₋₆ alkyl, C₁₋₆ alkoxy, C₃₋₆ cycloalkyl, C₃₋₆ cycloalkoxy, C₆-C₁₀ aryl, and 5-14 membered heteroaryl;

or X₈ is selected from CR₈, and R₈ is selected from the substituted or unsubstituted group consisting of halogen, CN, NH₂, ester, carbamido, carbamate, amido, C₁₋₆ alkyl, C₁₋₆ alkoxy, C₃₋₆ cycloalkyl, C₃₋₆ cycloalkoxy, C₆-C₁₀ aryl, and 5-14 membered heteroaryl;

when A is

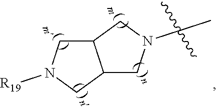

when both X₁ and X₂ are N, R₃ and X₄ form a substituted or unsubstituted 5-7 membered ring containing 0-3 heteroatoms selected from O, S, and N;

or R₁₁ and X₁₀ or X₁₂ form a substituted or unsubstituted 5-7 membered ring containing 0-3 heteroatoms selected from O, S, and N;

or R₁₀ and X₉ or X₁₁ form a substituted or unsubstituted 5-7 membered ring containing 0-3 heteroatoms selected from O, S, and N;

or R₅ and X₆ form a substituted or unsubstituted 5-7 membered ring containing 0-3 heteroatoms selected from O, S, and N;

or R₇ and X₈ form a substituted or unsubstituted 5-7 membered ring containing 0-3 heteroatoms selected from O, S, and N;

or R₁₃ and R₁₄ together with the P atom to which they are attached form a substituted or unsubstituted 4-8 membered heterocyclyl;

or B is selected from the group consisting of

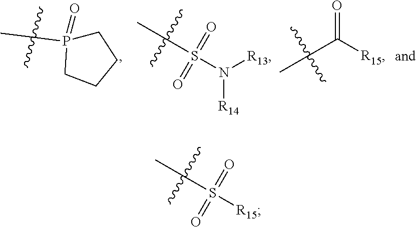

when A is

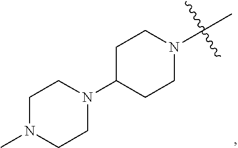 [SMILES: *N1CCC(N2CCN(C)CC2)CC1]

X₁ and X₂ are not N at the same time;

R₃ and X₂ or X₄ form a substituted or unsubstituted 5-7 membered ring containing 0-3 heteroatoms selected from O, S, and N;

or R₁₁ and X₁₀ or X₁₂ form a substituted or unsubstituted 5-7 membered ring containing 0-3 heteroatoms selected from O, S, and N;

or R₁₀ and X₉ or X₁₁ form a substituted or unsubstituted 5-7 membered ring containing 0-3 heteroatoms selected from O, S, and N;

or R₅ and X₆ form a substituted or unsubstituted 5-7 membered ring containing 0-3 heteroatoms selected from O, S, and N;

or R₇ and X₈ form a substituted or unsubstituted 5-7 membered ring containing 0-3 heteroatoms selected from O, S, and N;

or R₁₃ and R₁₄ together with the P atom to which they are attached form a substituted or unsubstituted 4-8 membered heterocyclyl;

or B is selected from the group consisting of

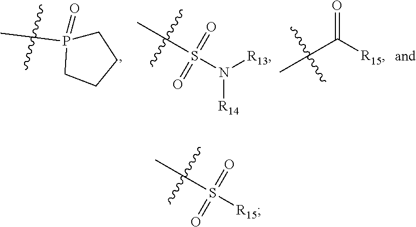

wherein, the substituted means being substituted by one or more groups selected from the group consisting of halogen, CN, OH, NH₂, ester, carbamido, carbamate, amido, C₁₋₆ alkyl, C₁₋₆ alkoxy, C₃₋₆ cycloalkyl, C₃₋₆ cycloalkoxy, C₂₋₆ alkenyl, C₂₋₆ alkynyl, C₆-C₁₀ aryl, 5-14 membered heteroaryl, and

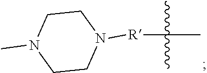

R′ is selected from the group consisting of C₁₋₆ alkylene, C₁₋₆ alkylene-CO—, and —CO—C₁₋₆ alkylene.

In another preferred embodiment, R₈ is a deuterated group or a halogenated group.

In another preferred embodiment, R₈ is deuterated C₁₋₆ alkoxy, deuterated C₁₋₆ alkyl, deuterated C₁₋₆ haloalkoxy, deuterated C₁₋₆ haloalkyl.

In another preferred embodiment, R₈ is selected from the group consisting of —O—CDF₂, —O—CD₃-, —O—CD₂F, —O—CF₃, —CD₃, —CDF₂, and —CD₂F.

In another preferred embodiment, R₁₁ and X₁₀ or X₁₂ form a substituted or unsubstituted 5-7 membered ring containing 0-3 heteroatoms selected from O, S, and N;

In another preferred embodiment, R₁₀ and X₉ or X₁₁ form a substituted or unsubstituted 5-7 membered ring containing 0-3 heteroatoms selected from O, S, and N.

In another preferred embodiment, R₅ and X₆ form a substituted or unsubstituted 5-7 membered ring containing 0-3 heteroatoms selected from O, S, and N.

In another preferred embodiment, R₇ and X₈ form a substituted or unsubstituted 5-7 membered ring containing 0-3 heteroatoms selected from O, S, and N.

In another preferred embodiment, R₁₃ and R₁₄ together with the P or N atom to which they are attached form a substituted or unsubstituted 4-8 membered heterocyclyl.

In another preferred embodiment, R₁₆ and R₁₇ together with the C atom to which they are attached form a substituted or unsubstituted 4-8 membered heterocyclyl.

In another preferred embodiment, R₁₁ and X₁₀ or X₁₂ form oxazolyl or imidazolyl.

In another preferred embodiment, R₃ and X₂ or X₄ form a substituted or unsubstituted 5-7 membered ring containing 0-3 heteroatoms selected from O, S, and N.

In another preferred embodiment, A and X₇ or X₆ form a substituted 5-7 membered ring, wherein, the substituted means that H on the 5-7 membered ring is substituted by one or more groups selected from the group consisting of halogen, CN, OH, NH₂, ester, carbamido, carbamate, amido, C₁₋₆ alkyl, C₁₋₆ alkoxy, C₃₋₆ cycloalkyl, C₃₋₆ cycloalkoxy, C₂₋₆ alkenyl, C₂₋₆ alkynyl, C₆-C₁₀ aryl, 5-14 membered heteroaryl, and

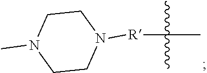

R′ is selected from the group consisting of C₁₋₆ alkylene, C₁₋₆ alkylene-CO—, and —CO—C₁₋₆ alkylene.

In another preferred embodiment, when A is

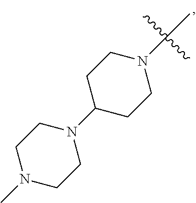 [SMILES: *N1CCC(N2CCN(C)CC2)CC1]

X₁ is CR₁ and/or X₂ is CR₂;

or R₃ and X₂ or X₄ form a substituted or unsubstituted 5-7 membered ring containing 0-3 heteroatoms selected from O, S, and N; R₁₁ and X₁₀ or X₁₂ form a substituted or unsubstituted 5-7 membered ring containing 0-3 heteroatoms selected from O, S, and N; R₁₀ and X₉ or X₁₁ form a substituted or unsubstituted 5-7 membered ring containing 0-3 heteroatoms selected from O, S, and N; wherein, the substituted means being substituted by one or more groups selected from the group consisting of halogen, CN, OH, NH₂, ester, carbamido, carbamate, amido, C₁₋₆ alkyl, C₁₋₆ alkoxy, C₃₋₆ cycloalkyl, C₃₋₆ cycloalkoxy, C₂₋₆ alkenyl, C₂₋₆ alkynyl, C₆-C₁₀ aryl, 5-14 membered heteroaryl, and

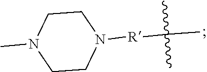

R′ is selected from the group consisting of C₁₋₆ alkylene, C₁₋₆ alkylene-CO—, and —CO—C₁₋₆ alkylene.

In another preferred embodiment, the compound has the structure of formula II, formula II′, formula III, formula IV or formula V

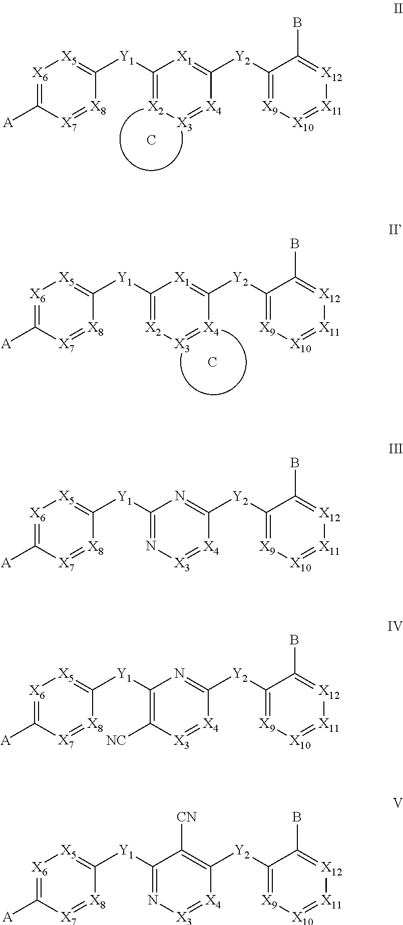

wherein,

ring C is a substituted or unsubstituted 5-7 membered ring;

X₁, X₂, X₃, X₄, X₅, X₆, X₇, X₈, X₉, X₁₀, X₁₁, X₁₂, Y₁, Y₂, A and B are as defined above.

with the proviso that

in formula III, when A is

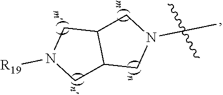

R₁₁ and X₁₀ or X₁₂ form a substituted or unsubstituted 5-7 membered ring containing 0-3 heteroatoms selected from O, S, and N; or X₃ and X₄ are each independently selected from N; or X₃ is CR₃, X₄ is CR₄, wherein, R₃ and R₄ are each independently selected from the group consisting of halogen, CN, NH₂, ester, carbamido, carbamate, amido, C₁₋₆ alkyl, C₁₋₆ alkoxy, C₃₋₆ cycloalkyl, C₃₋₆ cycloalkoxy, sulfonamido, amino, 3-10 membered heterocyclyl, C₆-C₁₀ aryl, and 5-14 membered heteroaryl; or R₃ and R₄ form a substituted or unsubstituted 5-7 membered ring containing 0-3 heteroatoms selected from O, S, and N; wherein, the substituted means being substituted by one or more groups selected from the group consisting of halogen, CN, OH, NH₂, ester, carbamido, carbamate, amido, C₁₋₆ alkyl, C₁₋₆ alkoxy, C₃₋₆ cycloalkyl, C₃₋₆ cycloalkoxy, C₂₋₆ alkenyl, C₂₋₆ alkynyl, C₆-C₁₀ aryl, and 5-14 membered heteroaryl; wherein, R₁₉, m, n, m′, n′ and R₁₁ are as defined above.

In another preferred embodiment, the compound has a structure of formula II or III, ring C is a substituted or unsubstituted 5-membered, 6-membered or 7-membered ring.

In another preferred embodiment, the ring C is saturated or unsaturated.

In another preferred embodiment, the ring C is aromatic or non-aromatic.

In another preferred embodiment, ring C is selected from the group consisting of substituted or unsubstituted C5, C6, or C7 cycloalkyl; substituted or unsubstituted 5 membered, 6 membered or 7 membered heterocyclyl; substituted or unsubstituted 5 membered or 6 membered heteroaryl; or C6 aryl, wherein, the heterocyclyl or heteroaryl has 1-3 heteroatoms selected from N, S and O, wherein, the “substituted” means being substituted by one or more groups selected from the group consisting of halogen, CN, OH, NH₂, ester, carbamido, carbamate, amido, C₁₋₆ alkyl, C₁₋₆ alkoxy, C₃₋₆ cycloalkyl, C₃₋₆ cycloalkoxy, C₂₋₆ alkenyl, C₂₋₆ alkynyl, C₆-C₁₀ aryl, and 5-14 membered heteroaryl.

In another preferred embodiment, the compound has the structure of formula X or formula XI

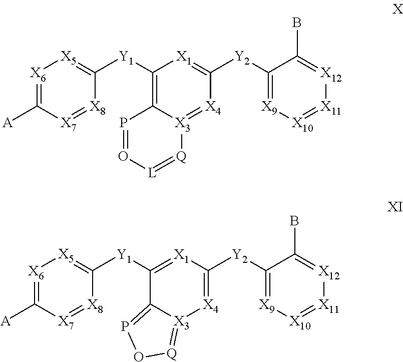

wherein, in formula X, O, P, Q and L are each independently selected from N or CR₁;

in formula XI, P and Q are each independently selected from N or CR₁, O is independently selected from N, O, S or CR₁;

X₁, X₃, X₄, X₅, X₆, X₇, X₈, X₉, X₁₀, X₁₁, X₁₂, Y₁, Y₂, A and B are as defined above.

In another preferred embodiment, the compound has the structure of formula VI, formula VII, formula VIII, or formula IX,

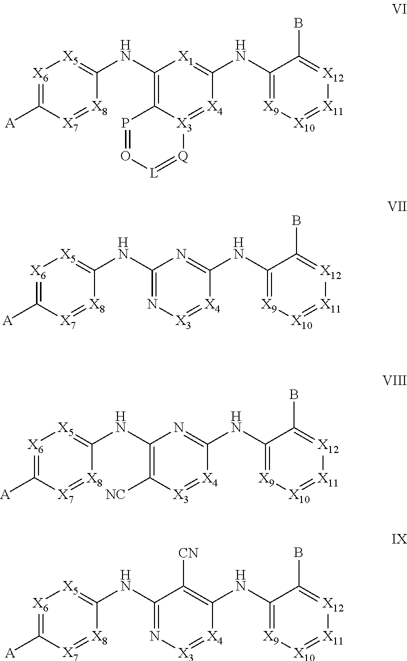

wherein, O, P, Q and L are each independently selected from N or CR₁;

with the proviso that in formula VII, when A is

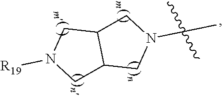

R₁₁ and X₁₀ or X₁₂ form a substituted or unsubstituted 5-7 membered ring containing 0-3 heteroatoms selected from O, S, and N; or R₃ and X₄ form a substituted or unsubstituted 5-7 membered ring containing 0-3 heteroatoms selected from O, S, and N;

wherein, the substituted means being substituted by one or more groups selected from the group consisting of halogen, CN, OH, NH₂, ester, carbamido, carbamate, amido, C₁₋₆ alkyl, C₁₋₆ alkoxy, C₃₋₆ cycloalkyl, C₃₋₆ cycloalkoxy, C₂₋₆ alkenyl, C₂₋₆ alkynyl, C₆-C₁₀ aryl, and 5-14 membered heteroaryl;

wherein, X₁, X₃, X₄, X₅, X₆, X₇, X₈, X₉, X₁₀, X₁₁, X₁₂, R₁₁, R₁₉, A, B, m, n, m′ and n′ are as defined above.

In another preferred embodiment, X₁, X₃, X₄, X₅, X₆, X₇, X₈, X₉, X₁₀, X₁₁, X₁₂, R₁₁, R₁₉, A, B, Y₁ and Y₂ are the specific group corresponding to each specific compound in the EXAMPLE.

In another preferred embodiment, the compound is selected from the group consisting of

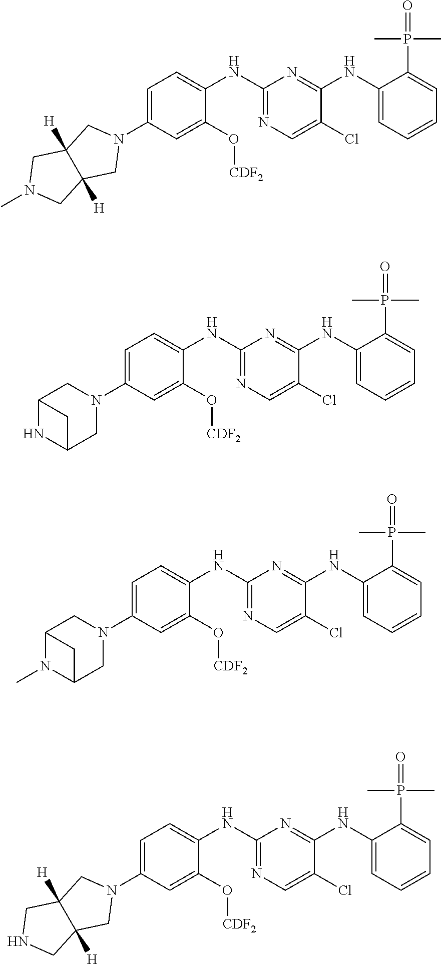

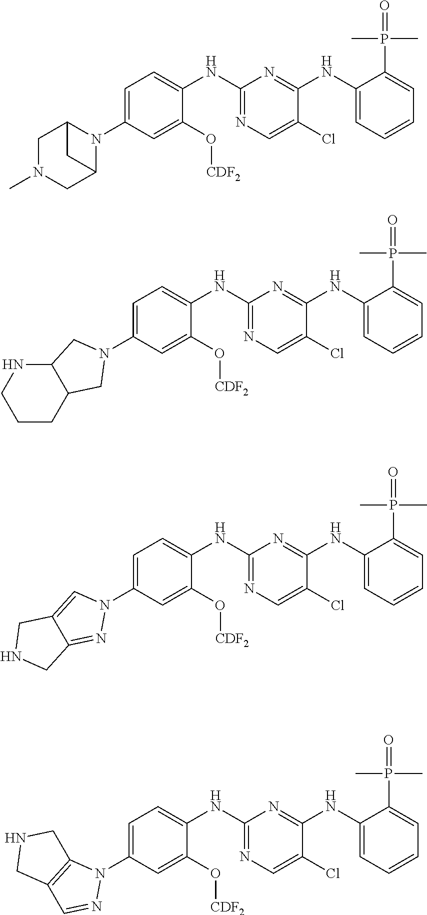

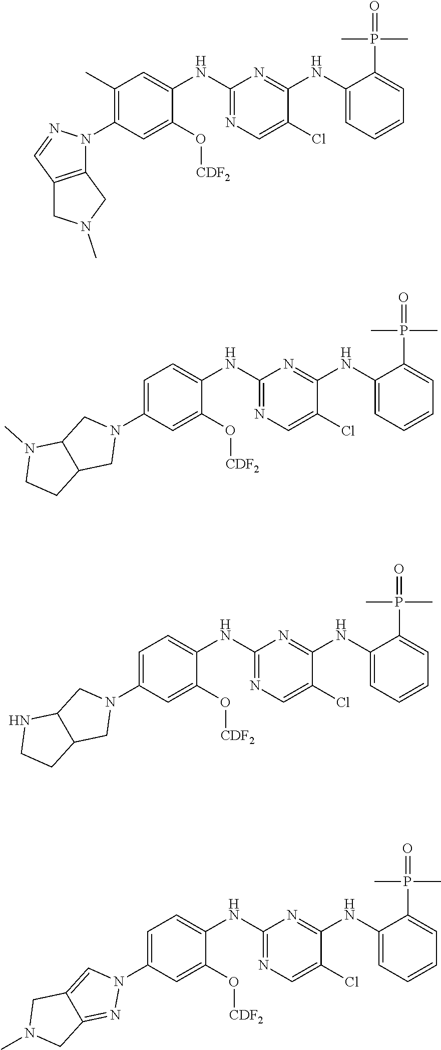

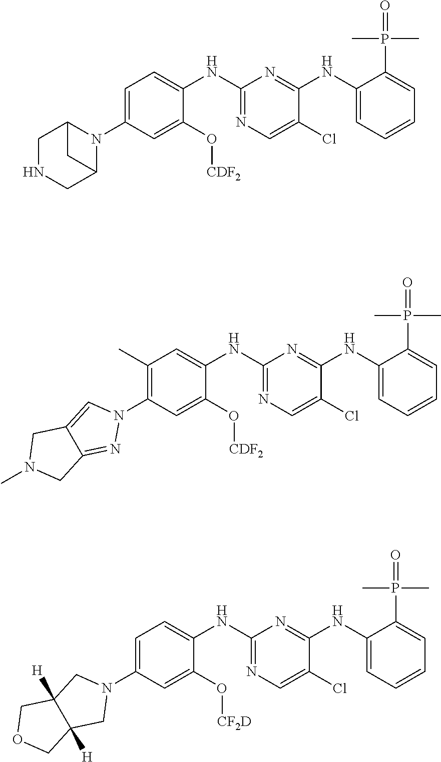

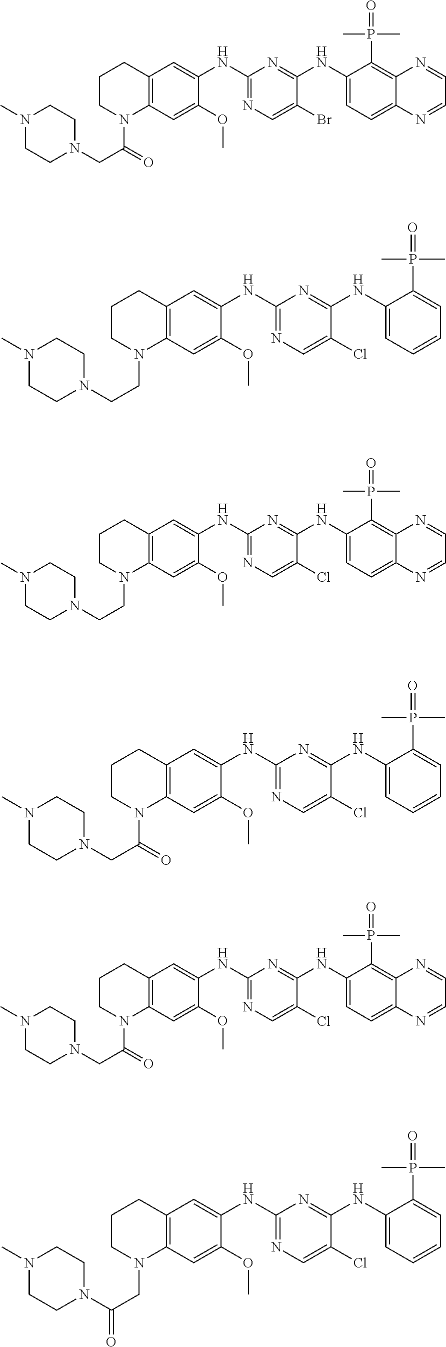

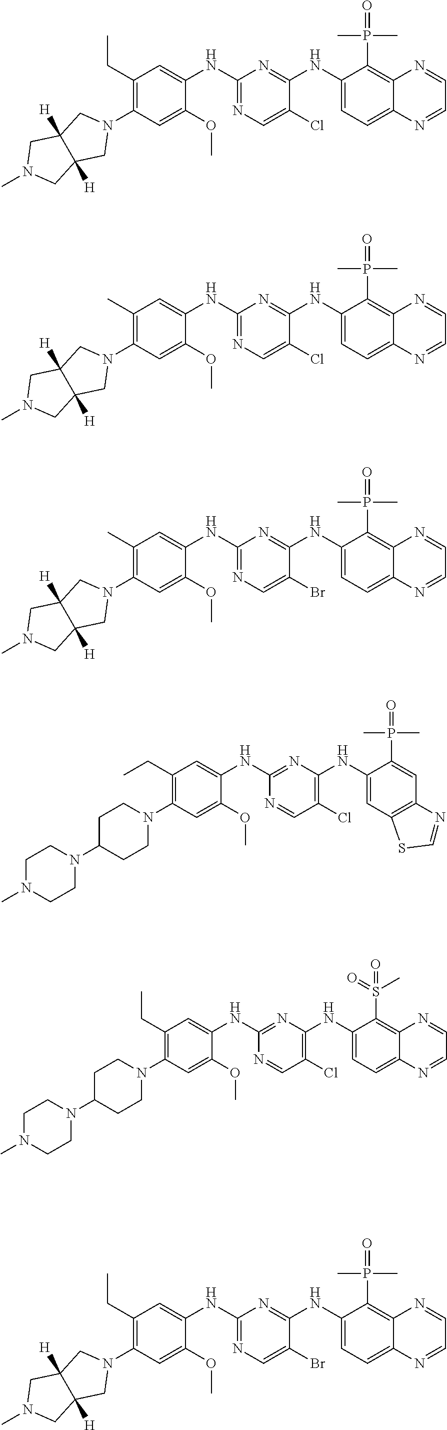 [SMILES: CCc1cc(Nc2ncc(Cl)c(Nc3ccc4nccnc4c3P(C)(C)=O)n2)c(OC)cc1N1C[C@H]2CN(C)C[C@H]2C1 ; COc1cc(N2C[C@H]3CN(C)C[C@H]3C2)c(C)cc1Nc1ncc(Cl)c(Nc2ccc3nccnc3c2P(C)(C)=O)n1 ; COc1cc(N2C[C@H]3CN(C)C[C@H]3C2)c(C)cc1Nc1ncc(Br)c(Nc2ccc3nccnc3c2P(C)(C)=O)n1 ; CCc1cc(Nc2ncc(Cl)c(Nc3cc4scnc4cc3P(C)(C)=O)n2)c(OC)cc1N1CCC(N2CCN(C)CC2)CC1 ; CCc1cc(Nc2ncc(Cl)c(Nc3ccc4nccnc4c3S(C)(=O)=O)n2)c(OC)cc1N1CCC(N2CCN(C)CC2)CC1 ; CCc1cc(Nc2ncc(Br)c(Nc3ccc4nccnc4c3P(C)(C)=O)n2)c(OC)cc1N1C[C@H]2CN(C)C[C@H]2C1]

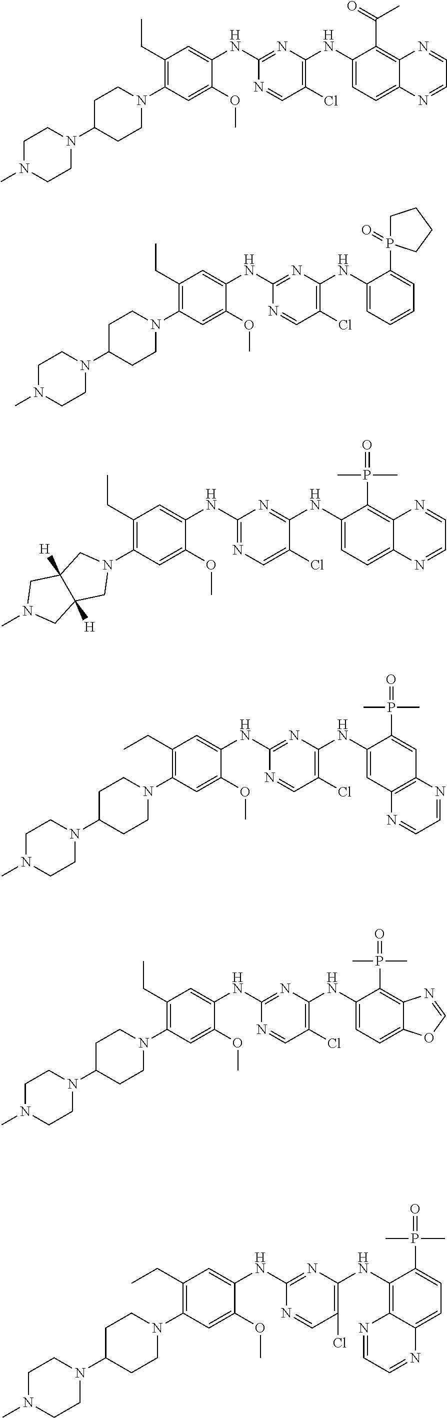

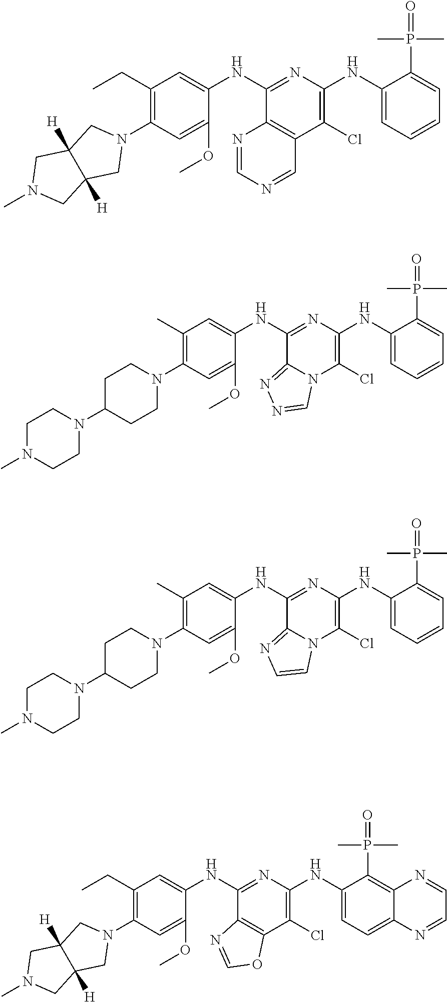

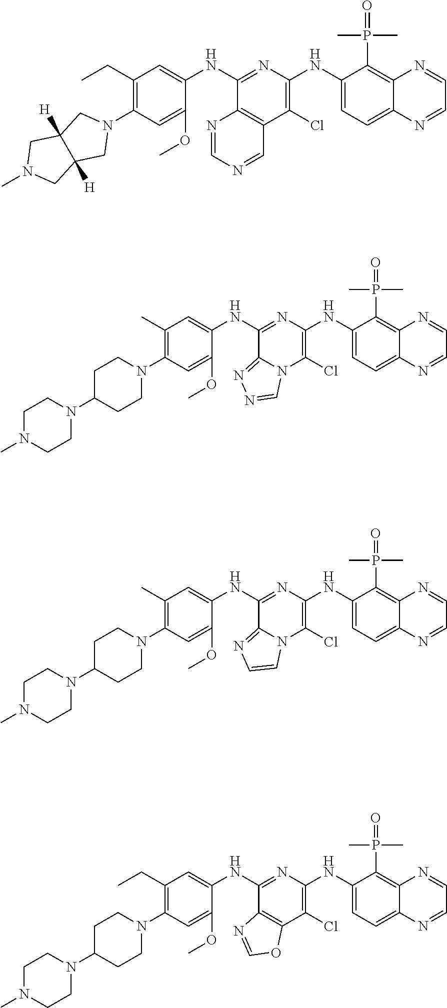 [SMILES: CCc1cc(Nc2nc(Nc3ccc4nccnc4c3P(C)(C)=O)c(Cl)c3cncnc23)c(OC)cc1N1C[C@H]2CN(C)C[C@H]2C1 ; COc1cc(N2CCC(N3CCN(C)CC3)CC2)c(C)cc1Nc1nc(Nc2ccc3nccnc3c2P(C)(C)=O)c(Cl)n2cnnc12 ; COc1cc(N2CCC(N3CCN(C)CC3)CC2)c(C)cc1Nc1nc(Nc2ccc3nccnc3c2P(C)(C)=O)c(Cl)n2ccnc12 ; CCc1cc(Nc2nc(Nc3ccc4nccnc4c3P(C)(C)=O)c(Cl)c3ocnc23)c(OC)cc1N1CCC(N2CCN(C)CC2)CC1]

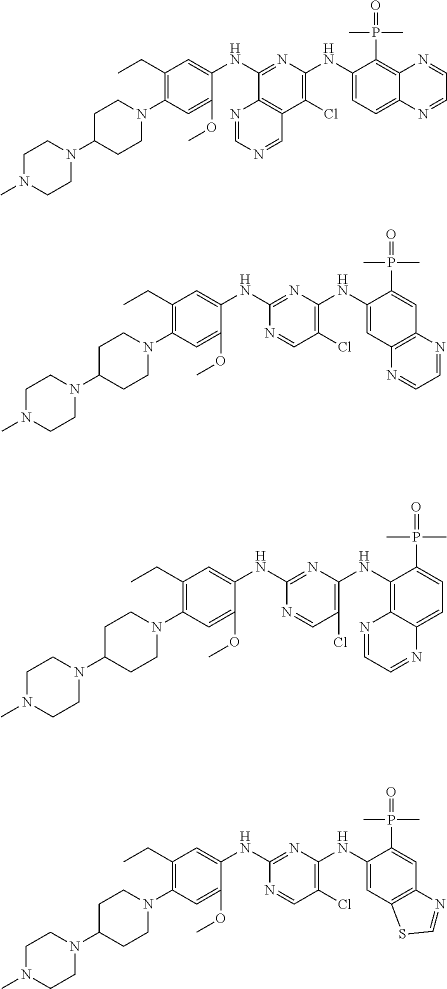

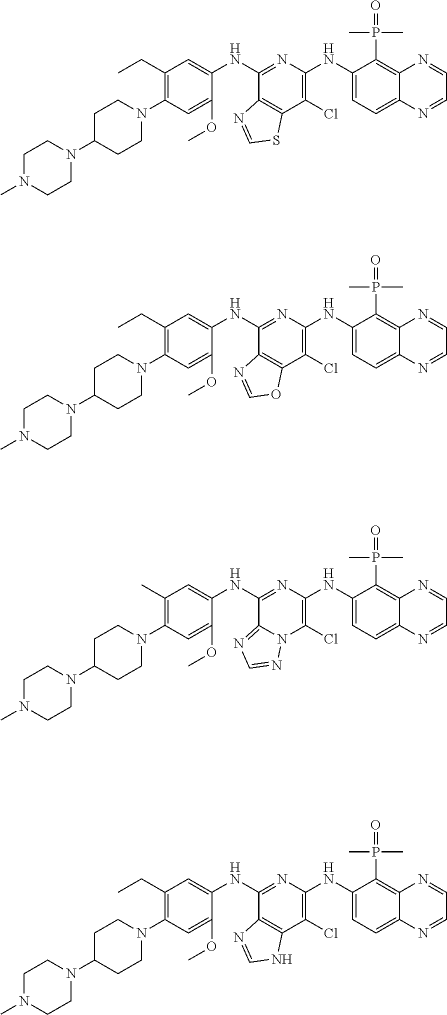 [SMILES: CCc1cc(Nc2nc(Nc3ccc4nccnc4c3P(C)(C)=O)c(Cl)c3scnc23)c(OC)cc1N1CCC(N2CCN(C)CC2)CC1 ; CCc1cc(Nc2nc(Nc3ccc4nccnc4c3P(C)(C)=O)c(Cl)c3ocnc23)c(OC)cc1N1CCC(N2CCN(C)CC2)CC1 ; COc1cc(N2CCC(N3CCN(C)CC3)CC2)c(C)cc1Nc1nc(Nc2ccc3nccnc3c2P(C)(C)=O)c(Cl)n2ncnc12 ; CCc1cc(Nc2nc(Nc3ccc4nccnc4c3P(C)(C)=O)c(Cl)c3[nH]cnc23)c(OC)cc1N1CCC(N2CCN(C)CC2)CC1]

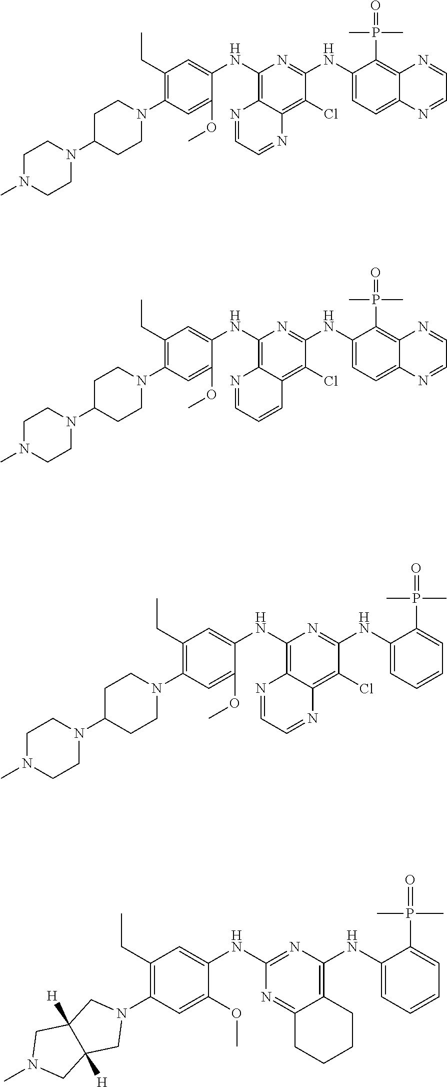

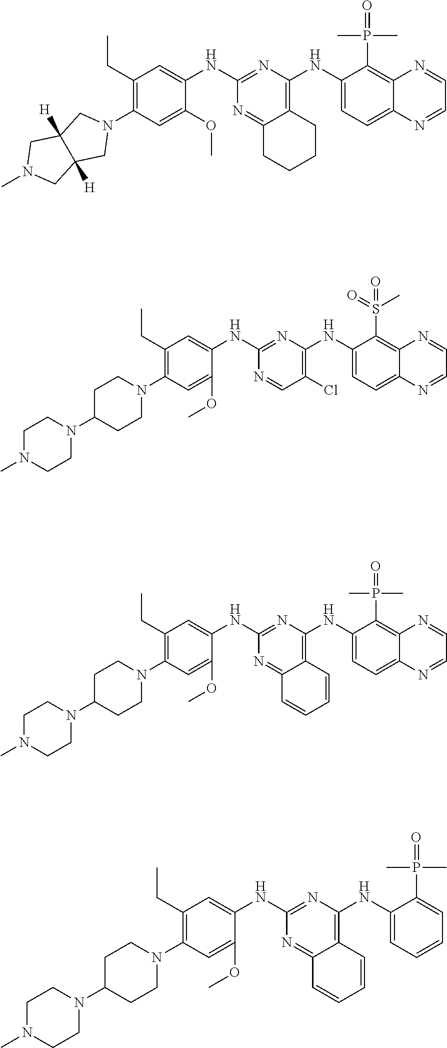

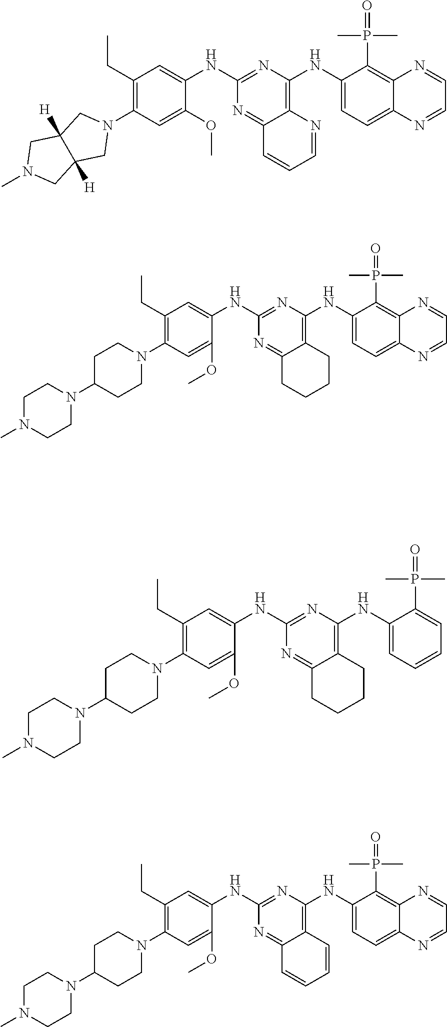

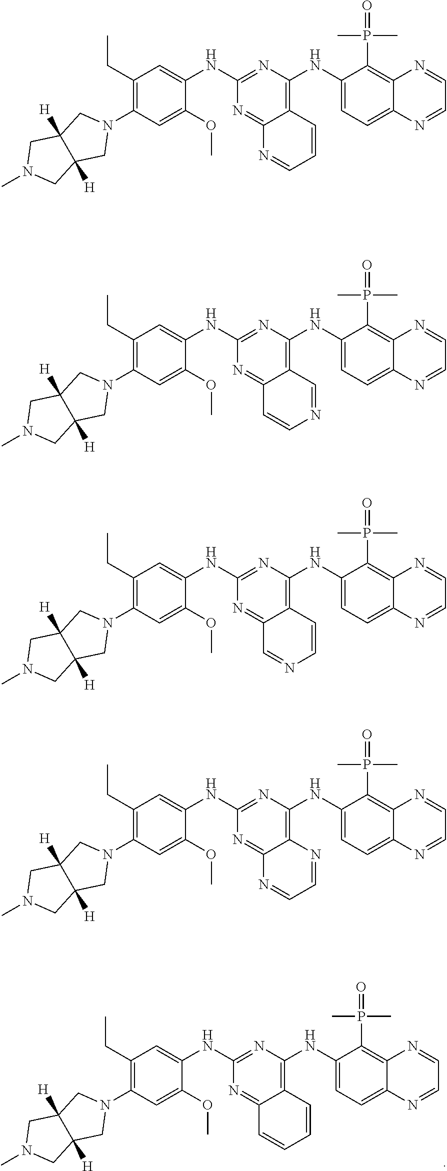 [SMILES: CCc1cc(Nc2nc(Nc3ccc4nccnc4c3P(C)(C)=O)c3cccnc3n2)c(OC)cc1N1C[C@H]2CN(C)C[C@H]2C1 ; CCc1cc(Nc2nc(Nc3ccc4nccnc4c3P(C)(C)=O)c3cnccc3n2)c(OC)cc1N1C[C@H]2CN(C)C[C@H]2C1 ; CCc1cc(Nc2nc(Nc3ccc4nccnc4c3P(C)(C)=O)c3ccncc3n2)c(OC)cc1N1C[C@H]2CN(C)C[C@H]2C1 ; CCc1cc(Nc2nc(Nc3ccc4nccnc4c3P(C)(C)=O)c3nccnc3n2)c(OC)cc1N1C[C@H]2CN(C)C[C@H]2C1 ; CCc1cc(Nc2nc(Nc3ccc4nccnc4c3P(C)(C)=O)c3ccccc3n2)c(OC)cc1N1C[C@H]2CN(C)C[C@H]2C1]

In another preferred embodiment, the compound is selected from the compounds shown in the EXAMPLE.

In the second aspect of the invention, it provides a pharmaceutical composition comprising the compound of the first aspect, or the pharmaceutically acceptable salt, the solvate or the prodrug thereof, and a pharmaceutically acceptable carrier.

In another preferred embodiment, it provides a preparation method of a pharmaceutical composition which comprises the step of mixing a pharmaceutically acceptable carrier with the compound of formula I, the pharmaceutically acceptable salt, the solvate or the prodrug thereof, thereby forming a pharmaceutical composition.

In another preferred embodiment, the pharmaceutical composition further comprises EGFR monoclonal antibody or MEK inhibitor.

In another preferred embodiment, the EGFR monoclonal antibody is selected from the group consisting of cetuximab, panitumumab, necitumumab, nimotuzumab, or a combination thereof.

In another preferred embodiment, the MEK inhibitor is selected from the group consisting of selumetinib, trametinib, PD0325901, U0126, Pimasertib (AS-703026), PD184352 (CI-1040), or a combination thereof.

In the third aspect of the invention, it provides a use of the compound of the first aspect, or the pharmaceutically acceptable salt, the solvate or the prodrug thereof in the preparation of a inhibitor or medicament for inhibiting mutant EGFR.

In another preferred embodiment, the mutant EGFR inhibitor is used for the treatment of cancer.

In another preferred embodiment, the cancer is selected from the group consisting of non-small cell lung cancer, small cell lung cancer, lung adenocarcinoma, lung squamous cell carcinoma, pancreatic cancer, breast cancer, prostate cancer, liver cancer, skin cancer, epithelial cell carcinoma, gastrointestinal stromal tumor, leukemia, histiocytic lymphoma, nasopharyngeal carcinoma, head and neck cancer, colon cancer, rectal cancer, glioma or a combination thereof.

In another preferred embodiment, the medicament is used to treat lung cancer caused by mutations in EGFR C797S.

In another preferred embodiment, the medicament is used to treat lung cancer caused by mutations in EGFRL858R/T790M/C797S.

In the fourth aspect of the present invention, it provides a method for treating cancer, which comprises the following steps: administering an effective amount of the above compound or the pharmaceutical composition to a subject in need thereof.

It should be understood that in the present invention, any of the technical features specifically described above and below (such as in the Example) can be combined with each other, thereby constituting new or preferred technical solutions which will not redundantly be described one by one herein.

### DETAILED DESCRIPTION OF THE INVENTION

After extensive and in-depth research, the inventors obtained a class of compounds that have a good inhibitory effect on EGFR (L858R/T790M/C797S) kinase, which can be used to prepare a medicament for regulating EGFR (L858R/T790M/C797S) kinase activity or treating EGFR (L858R/T790M/C797S) related diseases. On this basis, the inventor completed the present invention.

### Terms

In the present invention, unless otherwise specified, the terms used have the general meanings known to those skilled in the art.

The term “C₁₋₆ alkyl” refers to a linear or branched alkyl, including 1-6 carbon atoms, such as methyl, ethyl, propyl, isopropyl

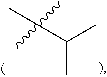 [SMILES: *C(C)C]

n-butyl, tert-butyl, isobutyl (such as

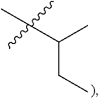 [SMILES: *C(C)CC]

n-pentyl, isopentyl, n-hexyl, isohexyl. “Substituted alkyl” refers to one or more positions in the alkyl are substituted, especially 1-4 substituents, which can be substituted at any position. Typical substituents include, but are not limited to one or more of the following groups: such as deuterium, halogen (such as monohalogenated substituent or polyhalogenated substituents, and the latter such as trifluoromethyl or alkyl containing Cl₃), cyano, nitro, oxo (such as ═O), trifluoromethyl, trifluoromethoxy, cycloalkyl, alkenyl, cycloalkenyl, alkynyl, heterocyclyl, aromatic ring, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}Rₑ, C(═O)OR_{d}, C(═O)Rₐ, C(═O)NR_{b}R_{c}, C(═O)Rₐ, C(═O)NR_{b}R_{c}, NR_{b}C(═O)ORₑ, NR_{d}C(═O)NR_{b}Rₑ, NR_{d}S(═O)₂NR_{b}R_{c}, NR_{d}P(═O)₂NR_{b}R_{c}, NR_{b}C(═O)Rₐ, or NR_{b}P(═O)₂R_{c}, wherein Rₐ can independently represent hydrogen, deuterium, alkyl, cycloalkyl, alkenyl, cycloalkenyl, alkynyl, heterocyclyl or aromatic ring, R_{b}, R_{c} and R_{d} can independently represent hydrogen, deuterium, alkyl, cycloalkyl, heterocyclyl or aromatic ring, or R_{b} and R_{c} together with the N atom form a heterocycle, R_{c} can independently represent hydrogen, alkyl, cycloalkyl, alkenyl, cycloalkenyl, alkynyl, heterocyclyl or aromatic ring. The above typical substituents, such as alkyl, cycloalkyl, alkenyl, cycloalkenyl, alkynyl, heterocyclyl or aromatic ring can be optionally substituted.

The term “alkylene” refers to a group formed by “alkyl” removing a hydrogen atom, such as methylene, ethylene, propylene, and isopropylene (such as

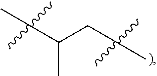 [SMILES: *CC(*)C]

butylene (such as

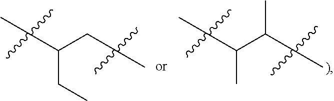

pentylidene (such as

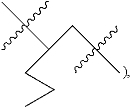 [SMILES: *CC(*)CCC]

hexylidene (such as

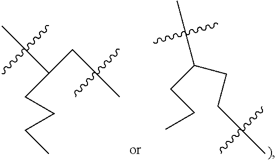

heptylene (such as

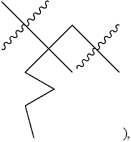 [SMILES: *CC(*)(C)CCCC]

etc. Among them, the H on alkylene can be substituted by alkyl, cycloalkyl, alkenyl, cycloalkenyl, alkynyl, heterocyclyl or aromatic ring.

The term “C₃₋₆ cycloalkyl” refers to a completely saturated cyclic hydrocarbon group, and each ring contains 3-6 carbon atoms. “Substituted cycloalkyl” refers to one or more positions in the cycloalkyl are substituted, especially 1-4 substituents, which can be substituted at any position. Typical substituents include, but are not limited to one or more of the following groups: such as deuterium, halogen (such as monohalogenated substituent or polyhalogenated substituent, and the latter such as trifluoromethyl or alkyl containing Cl₃), cyano, nitro, oxo (such as ═O), trifluoromethyl, trifluoromethoxy, cycloalkyl, alkenyl, cycloalkenyl, alkynyl, heterocyclyl, aromatic ring, ORₐ, SRₐ, S(═O)R_{c}, S(═O)₂R_{c}, P(═O)₂Rₑ, S(═O)₂OR_{c}, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}Rₑ, C(═O)OR_{d}, C(═O)Rₐ, C(═O)NR_{b}R_{c}, C(═O)Rₐ, C(═O)NR_{b}R_{c}, NR_{b}C(═O)OR_{c}, NR_{d}C(═O)NR_{b}R_{c}, NR_{d}S(═O)₂NR_{b}R_{c}, NR_{d}P(═O)₂NR_{b}Rₑ, NR_{b}C(═O)Rₐ, or NR_{b}P(═O)₂R_{c}, wherein Rₐ can independently represent hydrogen, deuterium, alkyl, cycloalkyl, alkenyl, cycloalkenyl, alkynyl, heterocyclyl or aromatic ring, R_{b}, R_{c} and R_{d} can independently represent hydrogen, deuterium, alkyl, cycloalkyl, heterocyclyl or aromatic ring, or R_{b} and R_{c} together with the N atom form a heterocycle, Rₑ can independently represent hydrogen, deuterium, alkyl, cycloalkyl, alkenyl, cycloalkenyl, alkynyl, heterocyclyl or aromatic ring. The above typical substituents can be optionally substituted. Typically substituent also includes spiro, bridged or fused ring, especially spiro cycloalkyl, spiro cycloalkenyl, spiro heterocyclyl (excluding heteroaryl ring), bridged cycloalkyl, bridged cycloalkenyl, bridged heterocyclyl (excluding heteroaryl ring), fused cycloalkyl, fused cycloalkenyl, fused heterocyclyl or fused aryl, the above cycloalkyl, cycloalkenyl, heterocyclyl and heteroaryl can be optionally substituted.

The term “3-10 membered heterocyclyl” refers to a completely saturated or partially unsaturated cyclic group (including monocyclic, bicyclic, or tricyclic system) in which at least one heteroatom is present in a ring having at least one carbon atom. Each heterocyclyl containing heteroatom can have 1, 2, 3 heteroatoms, and these heteroatoms are selected from nitrogen atom, oxygen atom or sulfur atom, wherein the nitrogen atom or sulfur atom can be oxidized, and the nitrogen atom can also be quaternized. 3-8 and 3-6 membered heterocyclyl have similar meanings. Heterocyclyl can be attached to the residue of any heteroatom or carbon atom of the ring or ring molecule. Typical monocyclic heterocyclyls include, but are not limited to azetidinyl, pyrrolidyl, oxetanyl, pyrazolinyl, imidazolinyl, imidazolidinyl, oxazolidinyl, isoxazolidinyl, thiazolidinyl, isothiazolidinyl, tetrahydrofuryl, piperidyl, piperazinyl, 2-oxoppiperazinyl, 2-oxopiperidyl, 2-oxopyrrolidyl, hexahydroacridheptyl, 4-piperidinone, tetrahydropyranyl, morpholinyl, thiomorpholinyl, thiomorpholinylsulfoxide, thiomorpholinylsulfone, 1,3-dioxane and tetrahydro-1,1-dioxythienyl, etc. The polycyclic heterocyclyl includes spiro, fused, and bridged heterocyclyls. The spiro, fused, and bridged heterocyclyls involved are optionally connected with other groups by single bond, or are further fused with other cycloalkyl, heterocyclyl, aryl and heteroaryl by any two or more atoms of the ring. The heterocyclyl may be substituted or unsubstituted, when being substituted, the substituent is preferably one or more of the following groups, independently selected from alkyl, deuterated alkyl, haloalkyl, alkoxy, haloalkoxy, alkenyl, alkynyl, alkylthio, alkylamino, halogen, amino, nitro, hydroxy, thiol, cyano, cycloalkyl, heterocyclyl, aryl, heteroaryl, cycloalkylthio, oxo, carboxy, and ester.

The term “C₆-C₁₀ aryl” refers to an aromatic cyclic hydrocarbons group, particularly monocyclic and bicyclic groups such as phenyl, biphenyl or naphthyl. Any aromatic ring having two or more aromatic rings (bicyclic, etc.), the aromatic rings of aryl may be connected by single bond (such as biphenyl) or fused (such as naphthalene, anthracene, etc.). “Substituted aryl” refers to one or more positions in the aryl are substituted, especially 1-3 substituents, which can be substituted at any position. Typical substituents include, but are not limited to one or more of the following groups: such as deuterium, halogen (such as monohalogenated substituent or polyhalogenated substituent, and the latter such as trifluoromethyl or alkyl containing Cl₃), cyano, nitro, oxo (such as ═O), trifluoromethyl, trifluoromethoxy, C₁₋₆ alkyl, C₃₋₆ cycloalkyl, C₃₋₆ cycloalkenyl, C₆-C₁₀ aryl, 3-10 membered heterocyclyl, ORₐ, SRₐ, S(═O)Rₑ, S(═O)₂Rₑ, P(═O)₂Rₑ, S(═O)₂ORₑ, P(═O)₂ORₑ, NR_{b}Rₑ, NR_{b}S(═O)₂Rₑ, NR_{b}P(═O)₂R_{c}, S(═O)₂NR_{b}R_{c}, P(═O)₂NR_{b}R_{c}, C(═O)OR_{d}, C(═O)Rₐ, C(═O)NR_{b}R_{c}, C(═O)Rₐ, C(═O)NR_{b}R_{c}, NR_{b}C(═O)OR_{c}, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein Rₐ present here can independently represent hydrogen, deuterium, C₁₋₆ alkyl or substituted C₁₋₆ alkyl, C₃₋₆ cycloalkyl or substituted C₃₋₆ cycloalkyl, C₃₋₆ cycloalkenyl or substituted C₃₋₆ cycloalkenyl, C₆-C₁₀ aryl or substituted C₆-C₁₀ aryl, 3-10 membered heterocyclyl or substituted 3-10 membered heterocyclyl, R_{b}, R_{c} and R_{d} can independently represent hydrogen, deuterium, C₁₋₆ alkyl or substituted C₁₋₆ alkyl, C₃₋₆ cycloalkyl or substituted C₃₋₆ cycloalkyl, C₃₋₆ cycloalkenyl or substituted C₃₋₆ cycloalkenyl, C₆-C₁₀ aryl or substituted C₆-C₁₀ aryl, 3-10 membered heterocyclyl or substituted 3-10 membered heterocyclyl, or R_{b} and R_{c} together with the N atom form a 3-14 membered heterocyclyl, Rₑ can independently represent hydrogen, deuterium, C₁₋₆ alkyl or substituted C₁₋₆ alkyl, C₃₋₆ cycloalkyl or substituted C₃₋₆ cycloalkyl, C₃₋₆ cycloalkenyl or substituted C₃₋₆ cycloalkenyl, C₆-C₁₀ aryl or substituted C₆-C₁₀ aryl, 3-10 membered heterocyclyl or substituted 3-10 membered heterocyclyl. The above typical substituents can be optionally substituted. Typical substituent also includes fused ring, especially fused cycloalkyl, fused cycloalkenyl, fused heterocyclyl or fused aryl, the above cycloalkyl, cycloalkenyl, heterocyclyl and heteroaryl can be optionally substituted.

The term “5-14 membered heteroaryl” refers to a heteroaromatic system containing 1-4 heteroatoms, 5-14 ring atoms, wherein the heteroatom is selected from oxygen, nitrogen and sulfur. The heteroaryl is preferably 5 to 10 membered, more preferably 5 or 6 membered, such as pyrryl, pyrazolyl, imidazolyl, oxazolyl, isoxazolyl, thiazoly, thiadiazolyl, isothiazolyl, furanyl, pyridyl, pyrazinyl, pyrimidyl, pyridazinyl, triazinyl, triazolyl, and tetrazolyl, etc. The “heteroaryl” may be substituted or unsubstituted, when being substituted, the substituent is preferably one or more of the following groups, independently selected from alkyl, deuterated alkyl, haloalkyl, alkoxy, haloalkoxy, alkenyl, alkynyl, alkylthio, alkylamino, halogen, amino, nitro, hydroxy, thiol, cyano, cycloalkyl, heterocyclyl, aryl, heteroaryl, cycloalkylthio, oxo, carboxy, and ester.

The term “C₁₋₆ alkoxy” refers to a straight or branched chain alkoxy having from 1 to 6 carbon atoms with a structure of C₁₋₆ alkyl-O—, wherein the alkyl is defined as above, and includes but not limited to methoxyl, ethoxyl, propoxyl, isopropoxyl and butoxyl, etc. Preferably C₁₋₃ alkoxy.

The term “C₃₋₆ cycloalkoxy” refers to a cyclic alkoxy having 3-6 carbon atoms. It includes cyclopropyloxy, cyclobutyloxy, cyclopentyloxy and cyclohexyloxy.

The term “C₁₋₆ alkoxycarbonyl” refers to C₁₋₆ alkoxy-C(═O)—.

The term “C₁₋₆ alkylcarbonyl” refers to C₁₋₆ alkyl-C(═O)—.

The term “halogen” or “halo” is chlorine, bromine, fluorine, and iodine.

The term “hydroxy” refers to a group with a structure of OH.

The term “ester” refers to a group with a structure of —COOR, wherein R represents hydrogen, C₁₋₆ alkyl or substituted C₁₋₆ alkyl, C₃₋₆ cycloalkyl or substituted C₃₋₆ cycloalkyl, C₃₋₆ cycloalkenyl or substituted C₃₋₆ cycloalkenyl, C₆-C₁₀ aryl or substituted C₆-C₁₀ aryl, 3-10 membered heterocyclyl or substituted 3-10 membered heterocyclyl.

The term “amino” refers to a group with a structure of —NRR′, wherein R and R′ can independently represent hydrogen, C₁₋₆ alkyl or substituted C₁₋₆ alkyl, C₃₋₆ cycloalkyl or substituted C₃₋₆ cycloalkyl, C₃₋₆ cycloalkenyl or substituted C₃₋₆ cycloalkenyl, C₆-C₁₀ aryl or substituted C₆-C₁₀ aryl, 3-10 membered heterocyclyl or substituted 3-10 membered heterocyclyl. R and R′ may be the same or different in the dialkylamino segment.

The term “amido” refers to a group with a structure of —CONRR′, wherein R and R′ can independently represent C₁₋₆ alkyl or substituted C₁₋₆ alkyl, C₃₋₆ cycloalkyl or substituted C₃₋₆ cycloalkyl, C₃₋₆ cycloalkenyl or substituted C₃₋₆ cycloalkenyl, C₆-C₁₀ aryl or substituted C₆-C₁₀ aryl, 3-10 membered heterocyclyl or substituted 3-10 membered heterocyclyl. R and R′ may be the same or different in the dialkylamino segment.

The term “sulfonamido” refers to a group with a structure of —SO₂NRR′, wherein R and R′ can independently represent hydrogen, C₁₋₆ alkyl or substituted C₁₋₆ alkyl, C₃₋₆ cycloalkyl or substituted C₃₋₆ cycloalkyl, C₂₋₆ cycloalkenyl or substituted C₂₋₆ cycloalkenyl, C₆-C₁₀ aryl or substituted C₆-C₁₀ aryl, 3-10 membered heterocyclyl or substituted 3-10 membered heterocyclyl. R and R′ may be the same or different in the dialkylamino segment.

The term “carbamido” refers to a group with a structure of —NRCONR′R″, wherein R, R′ and R″ can independently represent hydrogen, C₁₋₆ alkyl or substituted C₁₋₆ alkyl, C₃₋₆ cycloalkyl or substituted C₃₋₆ cycloalkyl, C₃₋₆ cycloalkenyl or substituted C₃₋₆ cycloalkenyl, C₆-C₁₀ aryl or substituted C₆-C₁₀ aryl, 3-10 membered heterocyclyl or substituted 3-10 membered heterocyclyl. R, R′ and R″ may be the same or different in the dialkylamine segment.

The term “carbamate” refers to a group with a structure of

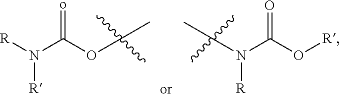

wherein R and R′ can independently represent hydrogen, C₁₋₆ alkyl or substituted C₁₋₆ alkyl, C₃₋₆ cycloalkyl or substituted C₃₋₆ cycloalkyl, C₃₋₆ cycloalkenyl or substituted C₃₋₆ cycloalkenyl, C₆-C₁₀ aryl or substituted C₆-C₁₀ aryl, 3-10 membered heterocyclyl or substituted 3-10 membered heterocyclyl. R and R′ may be the same or different in the dialkylamino segment.

In the present invention, the term “R₃ and X₂ or X₄ form a substituted or unsubstituted 5-7 membered ring containing 0-3 heteroatoms selected from O, S, and N” refers to when X₂ or X₄ is N, R₃ and its connected ring C atoms and adjacent N atoms form a substituted or unsubstituted 5-7 membered ring containing 0-3 heteroatoms selected from O, S, and N; when X₂ or X₄ is CR₂ or CR₃ respectively, R₃ and its connected ring C atom and CR₂ or CR₄ form a substituted or unsubstituted 5-7 membered ring containing 0-3 heteroatoms selected from O, S, and N. R₁₁ and X₁₀ or X₁₂ form a substituted or unsubstituted 5-7 membered ring containing 0-3 heteroatoms selected from O, S, and N, R₁₀ and X₉ or X₁₁ form a substituted or unsubstituted 5-7 membered ring containing 0-3 heteroatoms selected from O, S, and N, R₅ and X₆ form a substituted or unsubstituted 5-7 membered ring containing 0-3 heteroatoms selected from O, S, and N, and R₇ and X₈ form a substituted or unsubstituted 5-7 membered ring containing 0-3 heteroatoms selected from O, S, and N have similar meanings. 0-3 refers to the integers including 0, 1, 2, or 3.

5-7 refers to the integers including 5, 6, or 7.

The 5-7-membered ring comprises 5-7-membered cycloalkyl, 5-7-membered heterocyclyl, 5-7-membered aryl, 5-7-membered heteroaryl, such as cyclopentyl, cyclohexyl, tetrahydropyrrole, pyrrole, tetrahydrofuran, pyrazole, oxazole, imidazole, thiazole, isoxazole, isothiazole, phenyl, pyrazine, pyran, pyrimidine, pyridine, etc.

In the present invention, the term “substituted” refers to the substitution of one or more hydrogen atoms on a specific group by a specific substituent. The specific substituents are those described in the preceding paragraph or those present in each Example. Unless otherwise specified, a substituted group may have a substituent selected from a specific group at any substitutable position of the group, and the substituent may be the same or different at each position. Those skilled in the art should understand that the combinations of substituents contemplated by the present invention are those that are stable or chemically achievable. The substituent is such as (but is not limited to): deuterium, halogen, hydroxy, cyano, carboxy (—COOH), C1-C6 alkyl, C2-C6 alkenyl, C2-C6 alkynyl, C3-C8 cycloalkyl, 3- to 12 membered heterocyclyl, aryl, heteroaryl, C1-C8 aldehyde, C2-C10 acyl, C2-C10 ester, amino, C1-C6 alkoxy, C1-C10 sulfonyl, and C1-C6 uramido, etc.

Unless otherwise stated, it is assumed that any heteroatom with a lower valence state has enough hydrogen atoms to replenish its valence state.

When the substituent is a non-terminal substituent, it is a subunit of the corresponding substituent, such as alkyl corresponding to alkylene, cycloalkyl corresponding to cycloalkylene, heterocyclyl corresponding to heterocyclylene, alkoxy corresponding to alkyleneoxy, etc.

In the invention, the H on the compound or substituent group can be replaced by a deuterium atom.

Active Ingredient

As used herein, the terms “compounds of the invention” or “active ingredients of the invention” are used interchangeably and refer to compounds of formula I, or pharmaceutically acceptable salt, hydrate, solvate, isotope compound (e.g. deuterated compound) or prodrug thereof. The term also includes racemate and optical isomer.

The compound of formula I has the following structure:

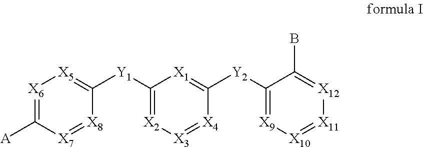

wherein, X₁, X₂, X₃, X₄, X₅, X₆, X₇, X₈, X₉, X₁₀, X₁₁, X₁₂, Y₁, Y₂, A and B are defined as above.

Preferably, in formula I, at least one of

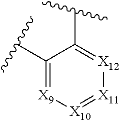

moiety,

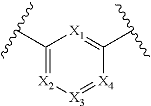

moiety or

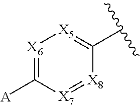

is a fused 9-10 membered bicycle.

Preferably, the compound of formula I has the structure of formula II, formula II′, formula III, formula IV or formula V,

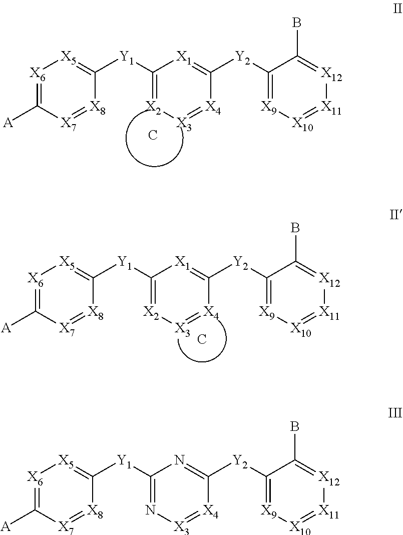

wherein,

ring C is a substituted or unsubstituted 5-7 membered ring;

with the proviso that

in formula III, when A is

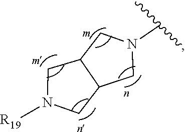

R₁₁ and X₁₀ or X₁₂ form a substituted or unsubstituted 5-7 membered ring containing 0-3 heteroatoms selected from O, S, and N; or X₃ and X₄ are each independently selected from N;

or X₃ is CR₃, X₄ is CR₄, wherein R₃ and R₄ are each independently selected from the group consisting of halogen, CN, NH₂, ester, carbamido, carbamate, amido, C₁₋₆ alkyl, C₁₋₆ alkoxy, C₃₋₆ cycloalkyl, C₃₋₆ cycloalkoxy, sulfonamido, amino, 3-10 membered heterocyclyl, C₆-C₁₀ aryl, 5-14 membered heteroaryl;

or R₃ and R₄ form a substituted or unsubstituted 5-7 membered ring containing 0-3 heteroatoms selected from O, S, and N;

wherein, the substituted means being substituted by one or more groups selected from the group consisting of halogen, CN, OH, NH₂, ester, carbamido, carbamate, amido, C₁₋₆ alkyl, C₁₋₆ alkoxy, C₃₋₆ cycloalkyl, C₃₋₆ cycloalkoxy, C₂₋₆ alkenyl, C₂₋₆ alkynyl, C₆-C₁₀ aryl, 5-14 membered heteroaryl;

wherein, X₁, X₂, X₃, X₄, X₅, X₆, X₇, X₈, X₉, X₁₀, X₁₁, X₁₂, Y₁, Y₂, A, B, R₁₉, m, n, m′, n′ and R₁₁ are defined as above.

Preferably, in formula I, R₃ and X₂ or X₄ form a substituted or unsubstituted 5-7 membered ring containing 0-3 heteroatoms selected from O, S, and N; and

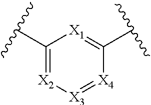

is not

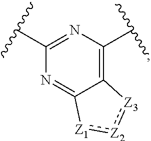

wherein, Z₁, Z₂ and Z₃ are each independently selected from CR₂₃, O, S, N or NR₂₃; each R₂₃ is independently H, C₁₋₆ alkyl; [symbol] is a single bond or a double bond;

wherein, the substituted means being substituted by one or more groups selected from the group consisting of halogen, CN, OH, NH₂, ester, carbamido, carbamate, amido, C₁₋₆ alkyl, C₁₋₆ alkoxy, C₃₋₆ cycloalkyl, C₃₋₆ cycloalkoxy, C₂₋₆ alkenyl, C₂₋₆ alkynyl, C₆-C₁₀ aryl, 5-14 membered heteroaryl,

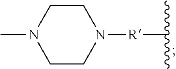

R′ is selected from the group consisting of C₁₋₆ alkylene, C₁₋₆ alkylene-CO—, —CO—C₁₋₆ alkylene.

Preferably, in formula I,

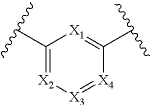

moiety is selected from

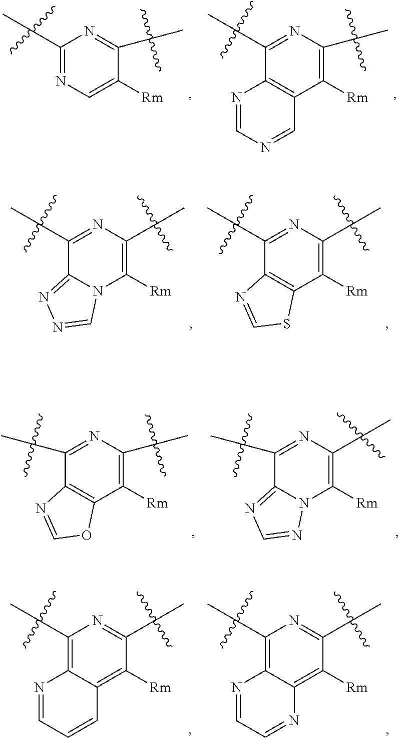

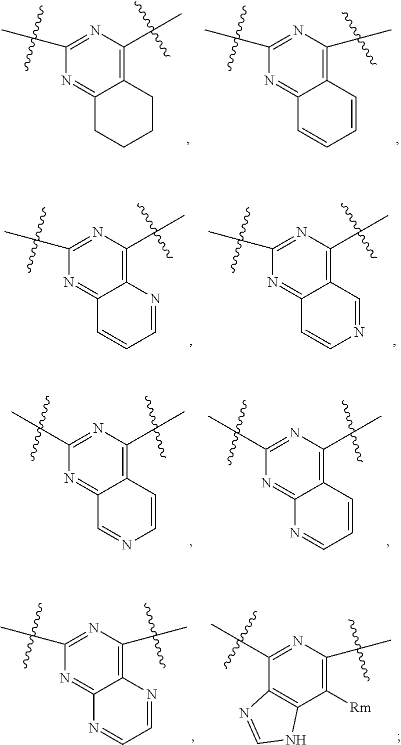

wherein Rm is halogen.

Preferably, in formula I, A is selected from

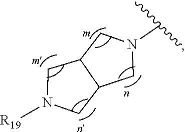

m, n, m′ and n′ are each independently selected from: 0, 1, 2 or 3, R₁₉ is selected from H, C₁₋₆ alkyl, C₁₋₆ alkoxy.

Preferably, in formula I,

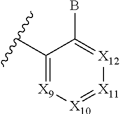

moiety is selected from

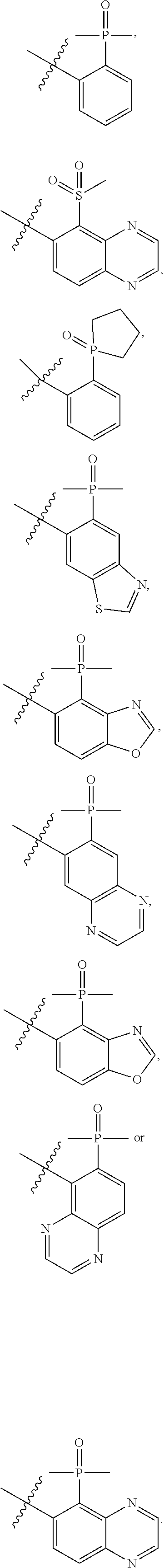

Preferably in formula I,

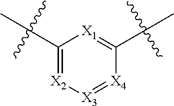

moiety is selected from

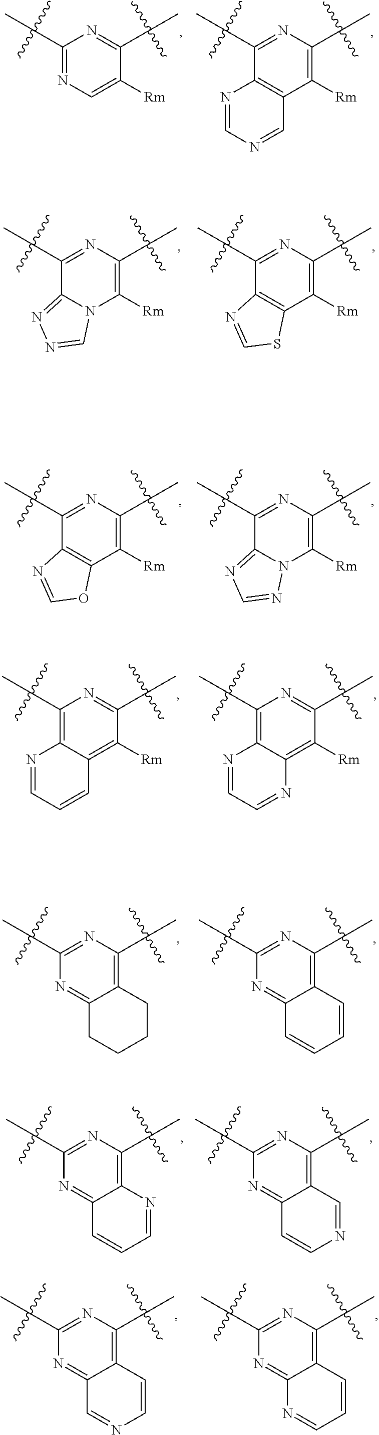

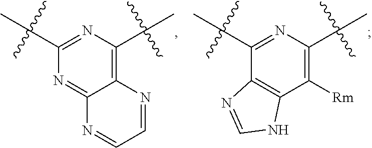

wherein Rm is halogen;

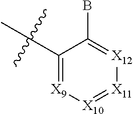

moiety is selected from

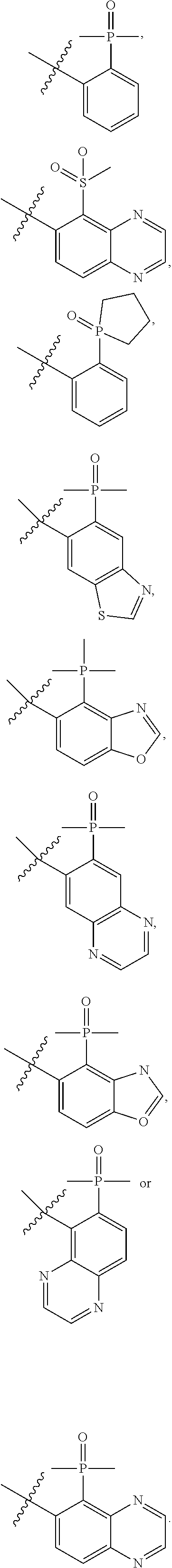

Preferably, in formula I,

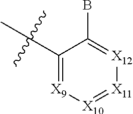

moiety is selected from

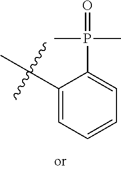 [SMILES: *c1ccccc1P(C)(C)=O]

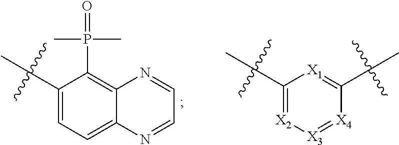

moiety is

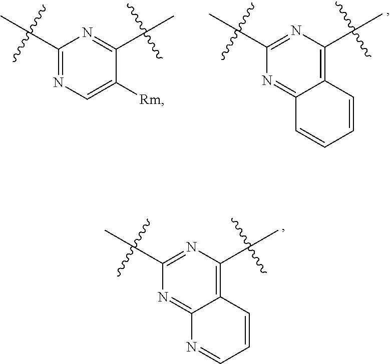

wherein, Rm is halogen.

Preferably, in the above each formula, both Y₁ and Y₂ are NH.

Preferably, R₈ is selected from the substituted or unsubstituted group consisting of H, halogen, CN, NH₂, ester, carbamido, carbamate, amido, C₁₋₆ alkyl and C₁₋₆ alkoxy; wherein the “substituted” means being substituted by one or more substituents selected from the group consisting of deuterium, halogen, CN, OH, NH₂, C₁₋₆ alkyl and C₁₋₆ alkoxy.

Preferably, R₆ is selected from the substituted or unsubstituted group consisting of C₁₋₆ alky and C₁₋₆ alkoxy; wherein the “substituted” means being substituted by one or more substituents selected from the group consisting of deuterium, halogen, CN, OH, NH₂, C₁₋₆ alkyl and C₁₋₆ alkoxy. The salt of the compound in the present invention that may be formed are also within the scope of the present invention. Unless otherwise stated, the compound in the present invention is understood to include its salt. The term “salt” as used herein refers to a salt formed in the form of acid or base from inorganic or organic acid and base. Further, when the compound in the present invention contains a base fragment, it includes, but is not limited to pyridine or imidazole, when it contains an acid segment, it includes, but is not limited to carboxylic acid. The zwitter-ion that may be formed (“inner salt”) is included within the scope of the term “salt”. Pharmaceutically acceptable (i.e., non-toxic, physiologically acceptable) salt is preferred, although other salts are also useful and may be used, for example, in the separation or purification steps of the preparation process. The compound of the present invention may form a salt, for example, compound I is reacted with a certain amount (such as an equivalent amount) of an acid or base, and precipitated in a medium, or freeze-dried in aqueous solution.

The base fragment contained in the compounds of the present invention includes but is not limited to amines or pyridine or imidazole rings, may form salt with organic or inorganic acid. Typical acids that form salts include acetate (such as acetate or trihalogenated acetic acid, such as trifluoroacetic acid), adipate, alginate, ascorbate, aspartate, benzoate, benzene sulfonate, disulfate, borate, butyrate, citrate, camphorate, camphor sulfonate, cyclopentane propionate, diethylene glycolate, lauryl sulfate, ethanesulphonate, fumarate, gluceptate, glycerophosphate, hemisulphate, enanthate, caproate, hydrochloride, hydrobromide, hydriodate, isethionate (e.g., 2-hydroxy-ethesulfonate), lactate, maleate, mesylate, naphthalenesulfonate (e.g., 2-naphthalenesulfonate), nicotinate, nitrate, oxalate, pectate, persulfate, phenylpropionate (e.g., 3-phenylpropionate), phosphate, picrate, pivalate, propionate, salicylate, succinate, sulfate (e.g., formed with sulfuric acid), sulfonate, tartrate, thiocyanate, toluenesulfonate (e.g., tosilate), dodecanoate, etc.

Some compounds of the invention may contain acidic fragments including, but not limited to carboxylic acid which may form salts with various organic or inorganic bases. Typical salt formed by base includes ammonium salt, alkali metal salt (such as sodium, lithium and potassium salts), alkaline earth metal salt (such as calcium and magnesium salts), and salt formed by organic bases (such as organic amines), such as benzathine, dicyclohexylamine, hydrabamine (salt formed with N,N-bis (dehydroabietyl) ethylenediamine), N-methyl-D-glucanamine, N-methyl-D-glucoamide, tert-butyllamine, and the salt formed with amino acids such as arginine, lysine, etc. Basic nitrogen-containing groups can form quaternary ammonium salts with halides, such as small molecular alkyl halides (such as chlorides, bromides and iodides of methyl, ethyl, propyl and butyl), dialkyl sulfate (such as dimethyl, diethyl, dibutyl, and dipentyl sulfates), long chain halides (such as chlorides, bromides and iodides of decyl, dodecyl, tetradecyl, and tetradecyl), aralkyl halides (such as bromides of benzyl and phenyl), etc.

The prodrug and solvate of the compound in the present invention are also included within the scope of the present invention. The term “prodrug” herein refers to a compound which will produce a compound, salt, or solvate of the present invention after chemical transformation of a metabolic or chemical process when it is used in the treatment of an associated disease. The compounds of the invention include solvates such as hydrates.

Compound, salt or solvate in the present invention, may be present in tautomeric forms such as amide and imino ether. All of these tautomers are part of the present invention.

Weight content of compound in the present invention obtained by preparation, separation and purification in turn is equal to or greater than 90%, such as equal to or greater than 95%, equal to or greater than 99% (“very pure” compound), and is listed in the description of the text. The “very pure” compound of the present invention is also part of the present invention.

In the entire specification, the groups and substituents can be selected to provide stable fragments and compounds.

The definitions of specific functional groups and chemical term are described as below in detail. For the purposes of the present invention, the chemical elements are consistent with Periodic Table of the Elements, CAS version, Handbook of Chemistry and Physics, 75^{th} Ed. The definition of a particular functional group is also described therein. In addition, the basic principles of Organic Chemistry as well as specific functional groups and reactivity are described in “Organic Chemistry”, Thomas Sorrell, University Science Books, Sausalito: 1999, the entire content of which is incorporated herein by reference.

The invention also includes isotope labeled compounds, which are equivalent to the original compounds disclosed herein. However, in practice, it usually occurs when one or more atoms are replaced by atoms with a different atomic weight or mass number. Examples of compound isotopes that may be listed in the present invention include hydrogen, carbon, nitrogen, oxygen, phosphorus, sulfur, fluorine and chlorine isotopes, such as ²H, ³H, ¹³C, ¹¹C, ¹⁴C, ¹⁵N, ¹⁸O, ¹⁷O, ³¹P, ³²P, ³⁵S, ¹⁸F and ³⁶Cl. The compound of the present invention, or the pharmaceutically acceptable salt, the solvate, or the prodrug thereof, and the compound containing isotopes or other isotope atoms of the above compound are all within the scope of the invention. Some isotope-labeled compounds in the present invention, such as the radioactive isotopes of ³H and ¹⁴C, are also included and are useful in experiments on the tissue distribution of drugs and substrates. Tritium (³H) and Carbon-14 (¹⁴C) are relatively easy to prepare and detect, and they are the preferred choice. In addition, heavier isotope substitutions such as deuterium, i.e. ²H, have advantages in certain therapies due to their good metabolic stability, such as increased half-life or reduced dosage in vivo, and thus may be preferred in certain situations. Isotope-labeled compounds can be prepared by conventional methods through substituting readily available isotope-labeled reagents for non-isotopic reagents by using the disclosed scheme shown in the Example.

When multiple locations in a particular structure are replaced by multiple specific substituents, the substituents at each location can be the same or different. The term “substituted” as used herein includes all substitution that allows organic compounds to be substituted. Broadly speaking, the allowable substituents include non-annular, cyclic, branched, non-branched, carbocyclic and heterocyclic, aromatic ring and non-aromatic organic compounds. In the present invention, such as heteroatom nitrogen, its valence state may be supplemented by a hydrogen substituent or by any permitted organic compound described above. Furthermore, the invention is unintentionally limited to the substituted organic compounds in any way. The present invention considers that a combination of substituents and variable groups is good for the treatment of diseases (such as infectious or hypertrophic diseases) in the form of stable compounds. The term “stable” herein refers to a stable compound which is sufficient for maintaining the integrity of the compound structure within a sufficiently long time, preferably be effective in a sufficiently long time, which is hereby used for the above purposes.

The metabolites of the compounds of the present application and their pharmaceutically acceptable salts, and prodrugs that can be converted into the compounds of the present application and their pharmaceutically acceptable salts thereof in vivo, are also included in the claims.

Preparation Method

### Route 1

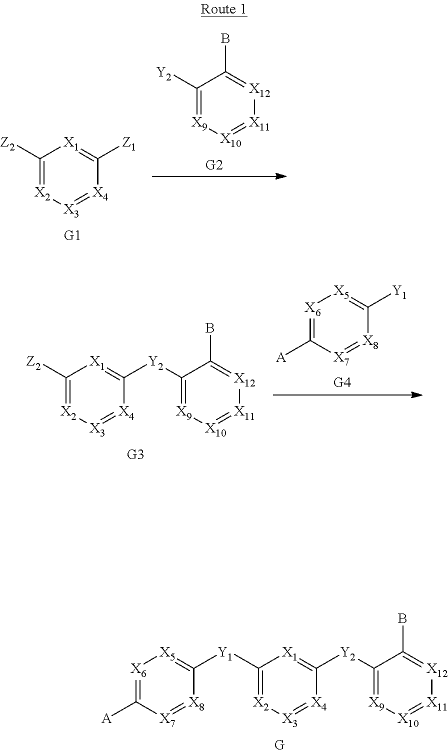

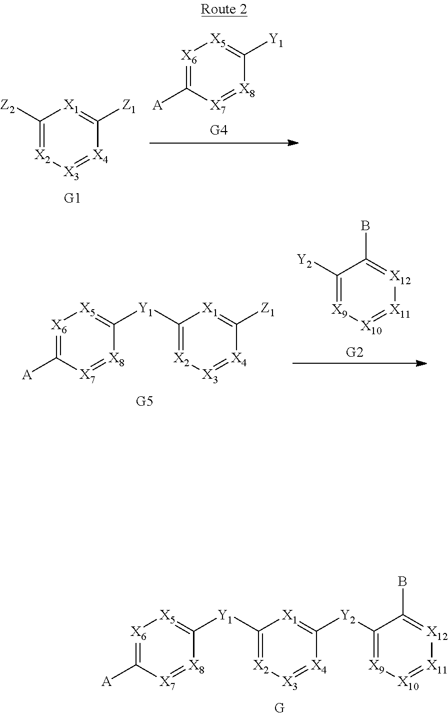

wherein, Z₁ and Z₂ are halogens

Route 1: Compound G1 and Compound G2 are reacted under the condition of acid or base or in the presence of appropriate catalyst and ligand to obtain Compound G3. Compound G3 and compound G4 are reacted under the condition of acid or base or in the presence of appropriate catalyst and ligand to obtain the target Compound G.

Route 2: Compound G1 and Compound G4 are reacted under the condition of acid or base or in the presence of appropriate catalyst and ligand to obtain Compound G5. Compound G5 and compound G2 are reacted under the condition of acid or base or in the presence of appropriate catalyst and ligand to obtain the target Compound G.

wherein, Z1, Z2 are halogens, X₁, X₂, X₃, X₄, X₅, X₆, X₇, X₈, X₉, X₁₀, X₁₁, X₁₂, Y₁, Y₂, A and B are defined as above.

Pharmaceutical Composition and Method of Administration

The compounds of the formula (I) may be used in combination with other drugs known to treat or improve similar conditions. When administered in combination, the original administration for the drug can remain unchanged, while compound of formula I may be administered simultaneously or subsequently. Pharmaceutical composition containing one or more known drugs and the compound of formula I may be preferred when administered in combination with one or more other drugs. The drug combination also includes administering the compound of formula I and other one or more known drugs at overlapping time. When the compound of formula I is combined with other one or more drugs, the dose of the compound or known drug may be lower than that of their individual use.

The dosage forms of the pharmaceutical composition of the present invention include (but are not limited to): injection, tablet, capsule, aerosol, suppository, pellicle, pill, liniment for external use, controlled release or sustained-release or nano formulation.

“Pharmaceutically acceptable carrier” refers to one or more compatible solid or liquid filler or gel substances, which are suitable for human use, and must be sufficiently pure and of sufficiently low toxicity. “Compatible” herein refers to each component of a composition can be mixed with the compound of the present invention and can be mixed with each other without appreciably reducing the efficacy of the compound. Examples of pharmaceutically acceptable carrier include cellulose and derivatives thereof (such as sodium carboxymethylcellulose, sodium ethylcellulose, cellulose acetate, etc.), gelatin, talc, solid lubricant (such as stearic acid, magnesium stearate), calcium sulfate, vegetable oil (such as soybean oil, sesame oil, peanut oil, olive oil, etc.), polyol (such as propylene glycol, glycerol, mannitol, sorbitol, etc.), emulsifier (such as Tween®), wetting agent (such as lauryl sodium sulfate), colorant, flavoring, stabilizer, antioxidant, preservative, pyrogen-free water, etc.

There is no special limitation of administration mode for the compound or pharmaceutical compositions of the present invention, and the representative administration mode includes (but is not limited to): oral, intratumorally, rectal, parenteral (intravenous, intramuscular or subcutaneous), and topical administration.

Solid dosage forms for oral administration include capsules, tablets, pills, powders and granules. In these solid dosage forms, the active compounds are mixed with at least one conventional inert excipient (or carrier), such as sodium citrate or dicalcium phosphate, or mixed with any of the following components: (a) fillers or compatibilizer, such as starch, lactose, sucrose, glucose, mannitol and silicic acid; (b) binders, such as hydroxymethyl cellulose, alginate, gelatin, polyvinylpyrrolidone, sucrose and arabic gum; (c) humectant, such as, glycerol; (d) disintegrating agent, such as agar, calcium carbonate, potato starch or tapioca starch, alginic acid, certain composite silicates, and sodium carbonate; (e) dissolution-retarding agents, such as paraffin; (f) absorption accelerators, such as quaternary ammonium compounds; (g) wetting agents, such as cetyl alcohol and glyceryl monostearate; (h) adsorbents, for example, kaolin; and (i) lubricants such as talc, calcium stearate, magnesium stearate, solid polyethylene glycol, lauryl sodium sulfate, or the mixtures thereof. In capsules, tablets and pills, the dosage forms may also contain buffering agents.

The solid dosage forms such as tablets, sugar pills, capsules, pills and granules can be prepared by using coating and shell materials, such as enteric coatings and any other materials known in the art. They can contain an opaque agent. The release of the active compounds or compounds in the compositions can be released in a delayed mode in a given portion of the digestive tract. Examples of the embedding components include polymers and waxes. If necessary, the active compounds and one or more above excipients can form microcapsules.

Liquid dosage forms for oral administration include pharmaceutically acceptable emulsions, solutions, suspensions, syrups or tinctures. In addition to the active compounds, the liquid dosage forms may contain any conventional inert diluents known in the art such as water or other solvents, solubilizers and emulsifiers, such as ethanol, isopropanol, ethyl carbonate, ethyl acetate, propylene glycol, 1,3-butanediol, dimethyl formamide, as well as oil, in particular, cottonseed oil, peanut oil, corn germ oil, olive oil, castor oil and sesame oil, or the combination thereof.

Besides these inert diluents, the composition may also contain additives such as wetting agents, emulsifiers, and suspending agent, sweetener, flavoring agents and perfume.

In addition to the active compounds, the suspension may contain suspending agent, for example, ethoxylated isooctadecanol, polyoxyethylene sorbitol and sorbitan esters, microcrystalline cellulose, methanol aluminum and agar, or the combination thereof.

The compositions for parenteral injection may comprise physiologically acceptable sterile aqueous or anhydrous solutions, dispersions, suspensions or emulsions, and sterile powders which can be re-dissolved into sterile injectable solutions or dispersions. Suitable aqueous and non-aqueous carriers, diluents, solvents or excipients include water, ethanol, polyols and any suitable mixtures thereof.

The dosage forms for topical administration of compounds of the invention include ointments, powders, patches, aerosol, and inhalants. The active ingredients are mixed with physiologically acceptable carriers and any preservatives, buffers, or propellant if necessary, under sterile conditions.

Compounds of the present invention can be administrated alone, or in combination with any other treatment means or therapeutic drugs.

When the pharmaceutical compositions are used, a safe and effective amount of compound of the present invention is administrated to a mammal (such as human) in need thereof, wherein the dose of administration is a pharmaceutically effective dose. For a person weighed 60 kg, the daily dose is usually 1-2000 mg, preferably 50-1000 mg. Of course, the particular dose should also depend on various factors, such as the route of administration, patient healthy status, which are well within the skills of an experienced physician.

The invention has the following main advantages:

(1) The compound of the invention has excellent inhibition ability to EGFR (C797S) kinase;

(2) The compound has better pharmacodynamics, pharmacokinetic properties and lower toxic and side effects.

The present invention will be further illustrated below with reference to the specific examples. It should be understood that these examples are only to illustrate the invention but not to limit the scope of the invention. The experimental methods without specific conditions in the following examples usually follow conventional conditions, such as Sambrook et al., Molecular Cloning: Laboratory Manual (New York: Cold Spring Harbor Laboratory Press, 1989), or according to the conditions recommended by the manufacturer. Unless otherwise stated, percentage and parts are calculated by weight.

All major and scientific terms used herein are the same as those familiar to those skilled in the art, unless otherwise defined. In addition, any method or material which is similar or equal to the recorded content may be applied to the method of the present invention. The preferred methods and materials described herein are described only for demonstration purposes.

The compound structure of the present invention was determined by nuclear magnetic resonance (NMR) and Liquid-mass chromatography (LC-MS).

NMR was detected by the Bruker AVANCE-400 NMR instrument, and the solvent included DMSO-d₆, CD₃COCD₃, CDCl₃ and CD₃OD, etc. The internal standard was tetramethylsilane (TMS), and the chemical shift was measured in percent per million (ppm).

LC-MS was detected by using Waters SQD2 mass spectrometry. HPLC was detected by using Agilent 1100 high voltage chromatograph (Microsorb 5 micron C18 100×3.0 mm column).

Qingdao GF254 silica gel plate was used for thin layer chromatography, 0.15-0.20 mm was used for TLC, and 0.9 mm-1 mm was used for preparative thin layer chromatography. Generally, Qingdao silica gel 200-300 mesh silica gel was used as carrier in column chromatography.

The starting materials in the Example of the present invention are known and commercially available, or may be synthesized by or in accordance with the literature reported in the art.

Unless otherwise specified, all reactions in the present invention are carried out by continuous magnetic stirring under the protection of dry inert gas (such as nitrogen or argon), and the reaction temperature is Celsius.

The preparation method of the compound of the formula (I) structure of the present invention is more specifically described below, but these specific methods do not constitute any limitation of the invention. The compound of the invention may also optionally be conveniently prepared by combining the various synthetic methods described in this specification or known in the art, such a combination may be easily performed by a skilled person in the art to which the invention belongs.

Typically, the preparation process for the compounds of the present invention is as follows, in which the raw materials and reagents used may be commercially purchased unless otherwise specified. The experimental methods without specific conditions in the following examples usually follow conventional conditions, or according to the conditions recommended by the manufacturer. Unless otherwise stated, percentage and parts are calculated by weight.

### EXAMPLE

### Example 1. Synthesis of Compound C1

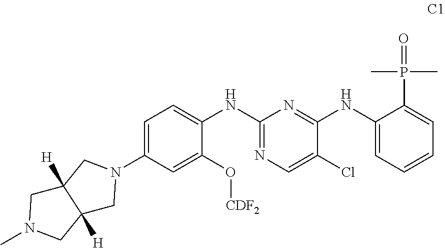

The experimental process was as follows:

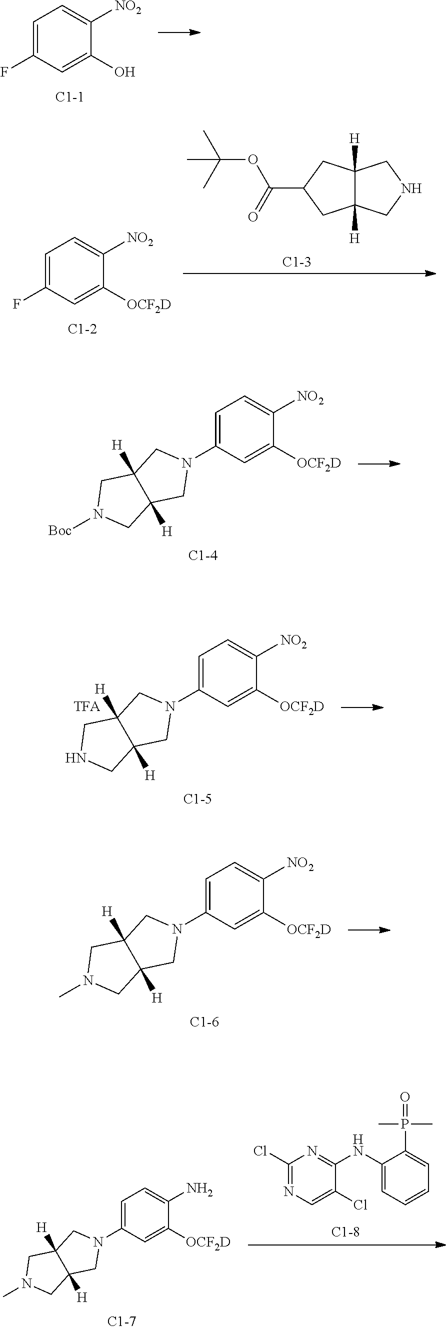

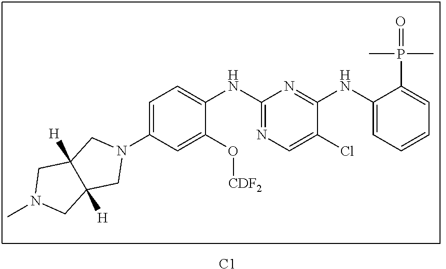

(1) Synthesis of C1-2

The Compound C1-1 (31.4 g, 0.2 mol) was dissolved in 600 ml of THF (dry), cooled to 0° C., NaH (80 g, 2 mol, 60% in oil) was added in batches, and reacted for 1 h. D₂O (200 g, 10 mol) was added at −10° C., and reacted at 0° C. for 1 h, then BrCF₂PO(OEt)₂ (106.8 g, 0.4 mol) was added dropwise at −10° C., and reacted at 5° C. for 0.5 h. TLC showed that the reaction was completed. Diluted with water, extracted with ethyl acetate, and the organic phases were combined, washed with saturated salt water, dried over anhydrous sodium sulfate, and concentrated to obtain a crude product, which was purified by column chromatography to obtain 40 g of the product. ¹H NMR (400 MHz, CDCl3) δ 7.96 (dd, J=9.1, 5.6 Hz, 1H), 7.05 (m, 2H), deuterium rate 99.2%.

(2) Synthesis of C1-4

The Compound C1-2 (700 mg, 3.37 mmol) and Compound C1-3 (928 mg, 4.38 mmol) were dissolved in DMF (15 ml), and anhydrous potassium carbonate (1.02 g, 7.40 mmol) was added, and then stirred overnight at 80° C. The solvent DMF was removed by evaporation under reduced pressure, the residue was diluted with water, extracted with ethyl acetate, and the combined organic phases were washed with saturated salt water, dried over anhydrous sodium sulfate, concentrated to obtain crude product which was purified by column chromatography to obtain 990 mg of Compound C1-4.

¹H NMR (400 MHz, CDCl₃) δ 8.06-8.04 (d, 1H, J=9.29 Hz), 6.39-636 (dd, 1H, J=9.32 Hz, 2.64 Hz), 6.32-6.31 (d, 1H, J=2.50 Hz), 3.67 (br, 4H), 3.38-3.27 (m, 4H), 3.07 (br, 2H), 1.46 (s, 9H).

(3) Synthesis of C1-5

The Compound C1-4 (350 mg, 0.77 mmol) was dissolved in trifluoroacetic acid (25 ml). After stirring at room temperature for 4 hours, the reaction solution was concentrated to give 665 mg of crude Compound C1-5 which was directly used for the next step without purification.

¹H NMR (400 MHz, CD₃OD) δ 7.95-7.92 (d, 1H, J=9.28 Hz), 6.51-6.48 (dd, 1H, J=9.26 Hz, 2.72 Hz), 6.36-6.35 (d, 1H, J=2.18 Hz), 3.58-3.50 (m, 4H), 3.42-3.39 (m, 2H), 3.25-3.19 (m, 4H).

(4) Synthesis of C1-6

The Compound C1-5 (665 mg, 1.68 mmol) was dissolved in methanol (20 ml), then 37% aqueous formaldehyde solution (544 mg, 6.71 mmol) and glacial acetic acid (403 mg, 6.71 mmol) were added, stirred at room temperature for 1 h, then sodium triacetoxyborohydride (1.8 g, 8.39 mmol) was added in batches and stirred overnight at room temperature. The reaction solution was concentrated, alkalized to pH >7 by adding saturated sodium bicarbonate solution, extracted with dichloromethane, washed with saturated salt water, dried over anhydrous sodium sulfate, concentrated to obtain crude product which was purified by column chromatography to obtain 302 mg Compound C1-6. Ms [M+H] 315.2.

(5) Synthesis of C1-7

The Compound C1-6 (302 mg, 0.96 mmol) was dissolved in methanol (10 ml), and Pd (OH)₂ (30 mg) was added, then stirred for 4h under hydrogen atmosphere. TLC detection showed the reaction was completed. The reaction solution was filtered through diatomite, washed with methanol, and the filtrate was concentrated to obtain 256 mg Compound C1-7.

(6) Synthesis of Compound C1

The Compound C1-7 (256 mg, 0.90 mmol) and Compound C1-8 (299 mg, 0.95 mmol) were dissolved in ethylene glycol monomethyl ether (25 ml), then 4MHCl/dioxane (0.68 ml, 2.70 mmol) was added, stirred overnight at 120° C., concentrated under reduced pressure, alkalized with saturated sodium bicarbonate solution, extracted with ethyl acetate. The combined organic phases were washed with saturated salt water, dried over anhydrous sodium sulfate, and concentrated to obtain crude product. Certain amount of the crude compound was purified by Prep-TLC to obtain 21 mg Compound CL. Ms[M+H] 564.2.

¹H NMR (400 MHz, CDCl₃) δ 10.91 (s, 1H), 8.59-8.56 (q, 1H), 8.07 (s, 1H), 7.99-7.97 (d, 1H, J=8.85 Hz), 7.46-7.42 (t, 1H), 7.31-7.25 (m, 1H), 7.13-7.09 (m, 1H), 6.92 (s, 1H), 6.54-6.48 (m, 2H), 3.32-3.20 (m, 8H), 2.66-2.60 (m, 5H), 1.85-1.81 (d, 6H, J=12.97 Hz).

### Example 2 Synthesis of Compound C2

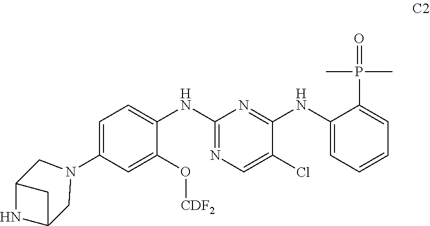

The experimental process was as follows:

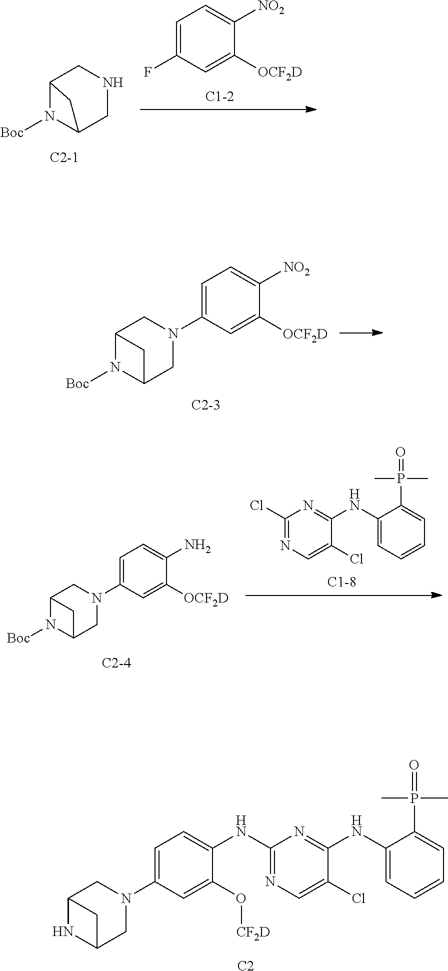

(1) Synthesis of C2-3

The Compound C2-1 (400 mg, 1.92 mmol) and Compound C1-2 (457 mg, 2.30 mmol) were dissolved in acetonitrile (10 ml), and potassium carbonate (530 mg, 3.84 mmol) was added, and then stirred overnight at 80° C. The solvent DMF was removed by evaporation under reduced pressure, the residue was diluted with water, extracted with ethyl acetate, and the combined organic phases were washed with saturated salt water, dried over anhydrous sodium sulfate, concentrated to obtain crude product which was purified by column chromatography to obtain 443 mg of Compound C2-3.

¹H NMR (400 MHz, CDCl₃) δ 8.10-8.07 (d, 1H, J=8.95 Hz), 6.61-6.58 (dd, 1H, J=9.37 Hz, 2.68 Hz), 6.54-6.53 (d, 1H, J=2.49 Hz), 4.33-4.32 (d, 2H, J=6.12 Hz), 3.38-3.35 (d, 2H, J=11.50 Hz), 2.75-2.69 (q, 1H), 1.51-1.49 (d, 1H, J=9.20 Hz), 1.36 (s, 9H).

(2) Synthesis of Compound C2-4

The Compound C2-3 (443 mg, 1.15 mmol) was dissolved in methanol (10 ml) and ethyl acetate (5 ml), Pd/C (45 mg) was added, and stirred overnight under hydrogen atmosphere. The reaction solution was filtered through diatomite, washed with methanol, and the filtrate was concentrated to obtain 367 mg Compound C2-4. Ms [M+H] 257.2.

(3) Synthesis of Compound C2

The Compound C2-4 (342 mg, 1.27 mmol) and Compound C1-8 (421 mg, 1.33 mmol) were dissolved in ethylene glycol monomethyl ether (15 ml), then TsOH (304 mg, 3.17 mmol) was added, stirred overnight at 90° C., concentrated under reduced pressure, alkalized with saturated sodium bicarbonate solution, extracted with ethyl acetate. The combined organic phases were washed with saturated salt water, dried over anhydrous sodium sulfate, and concentrated to obtain crude product which was purified by Prep-TLC to obtain 18 mg Compound C2. Ms [M+H] 536.2.

¹H NMR (400 MHz, CD₃OD) δ 8.37-8.34 (q, 1H), 8.03 (s, 1H), 7.60-7.55 (m, 2H), 7.47-7.43 (t, 1H), 7.24-7.20 (m, 1H), 6.70-6.66 (m, 2H), 4.52-4.51 (d, 1H, J=6.09 Hz), 3.90-3.87 (d, 2H, J=11.65 Hz), 3.81-3.78 (d, 2H, J=11.65 Hz), 3.09-3.03 (m, 1H), 2.02-2.00 (m, 1H), 1.86-1.83 (d, 6H, J=12.71 Hz).

### Example 3 Synthesis of Compound C3

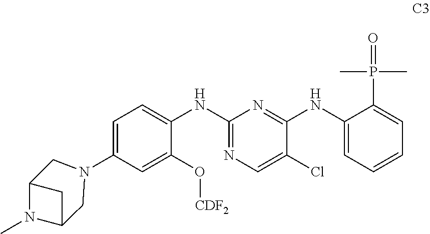

The experimental process was as follows:

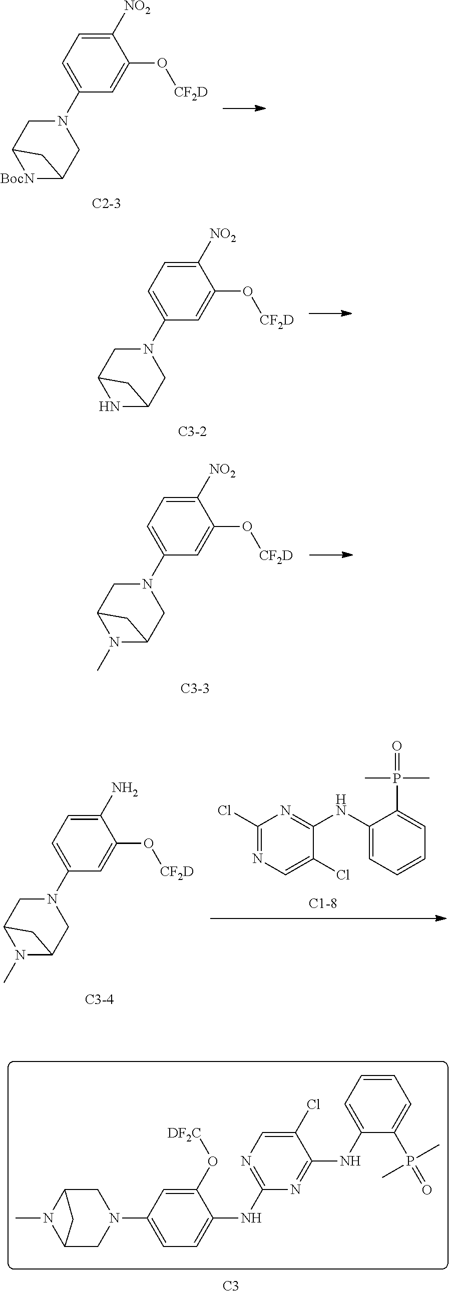

(1) Synthesis of C3-2

The Compound C2-3 (1.43 g) was dissolved in dichloromethane (20 ml), and trifluoroacetic acid (20 ml) was added and stirred at room temperature for 2 hours. After the reaction was completed, the reaction solution was concentrated under reduced pressure, and the residue was added with water and potassium carbonate, then the pH was adjusted to 9-10, extracted with dichloromethane for three times. The organic phase was washed with salt water, dried and concentrated to obtain 1.03 g of Compound C3-2.

¹HNMR (400 MHz, CDCl₃) δ8.09-8.07 (d, 1H, J=9.36), 6.60-6.57 (dd, 1H), 6.52-6.51 (d, 1H, J=2.6), 3.95-3.94 (d, 2H, J=5.96), 3.66-3.60 (m, 4H), 2.86-2.81 (m, 1H), 1.62-1.59 (d, 1H, J=9.08).

(2) Synthesis of C3-3

The Compound C3-2 (1.03 g), 37% aqueous formaldehyde solution (1.17 g) and acetic acid (0.86 g) were added into tetrahydrofuran (20 ml), and stirred at room temperature for 1 h. Sodium triacetoxyborohydride (3.82 g) was added in batches, and stirred overnight at room temperature. TLC showed that the reaction was completed. Water was added, and pH was adjusted to 9-10 with solid potassium carbonate, extracted with ethyl acetate. The organic phases were combined and washed with salt water, dried over anhydrous sodium sulfate, and concentrated to obtain 1.03 g of Compound C3-3;

¹H NMR (400 MHz, CDCl₃): δ 8.11-8.09 (d, 1H, J=9.32), 6.63-6.60 (dd, 1H), 6.56-6.55 (d, 1H, J=2.52), 3.75-3.74 (d, 2H, J=5.76), 3.67-3.64 (d, 2H, J=11.52), 3.40-3.37 (d, 2H, J=11.48), 2.74-2.69 (m, 1H), 2.20 (s, 3H), 1.61-1.59 (d, 1H, J=8.84.

(3) Synthesis of C3-4

The Compound C3-3 (1.03 g) was dissolved in methanol (25 ml), and palladium carbon (0.2 g) was added. The reaction was carried out in hydrogen for 15 hours, filtered, the filter cake was washed with methanol, and the filtrate was concentrated to obtain 1.02 g of Compound C3-4;

¹H NMR (400 MHz, CDCl₃): δ 6.81-6.79 (d, 1H, J=8.68), 6.50-6.49 (d, 1H, J=2.72), 6.48-6.46 (m, 1H), 3.71-3.70 (d, 1H, J=5.72), 3.27-3.24 (d, 1H, J=10.6), 2.65-2.60 (m, 1H), 2.14 (S, 3H), 1.65-1.63 (d, 1H, J=8.44).

(4) Synthesis of Compound C3

The Compound C3-4 (270 mg), Compound C1-8 (316 mg), and methanesulfonic acid (288 mg) were added to tert-butanol (10 ml) to react at 80° C. for 20h. After concentration, water was added, extracted with dichloromethane. The organic phase was washed with salt water, dried, concentrated and purified by column chromatography to obtain 151 mg of Compound C3;

¹H NMR (400 MHz, CDCl₃) δ10.90 (s, 1H), 8.62-8.59 (m, 1H), 8.07 (s, 1H), 7.92-7.89 (d, 1H, J=8.96), 7.45-7.41 (m, 1H), 7.30-7.24 (m, 1H), 7.11-7.07 (m, 1H), 6.80 (s, 1H), 6.61-6.58 (dd, 1H), 6.54-6.53 (d, 1H, J=2.48), 3.73-3.71 (d, 2H, J=5.84), 3.56-3.53 (d, 2H, J=10.84), 3.32-3.29 (d, 2H, J=10.76), 2.67-2.62 (m, 1H), 2.17 (s, 3H), 1.85 (s, 3H), 1.82 (s, 3H), 1.64-1.62 (d, 1H, J=8.56).

### Example 4 Synthesis of Compound C4

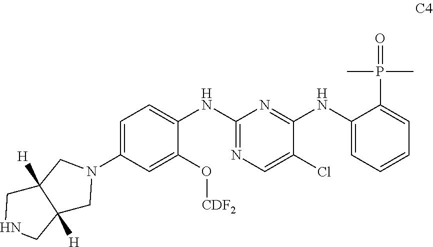

The experimental process was as follows:

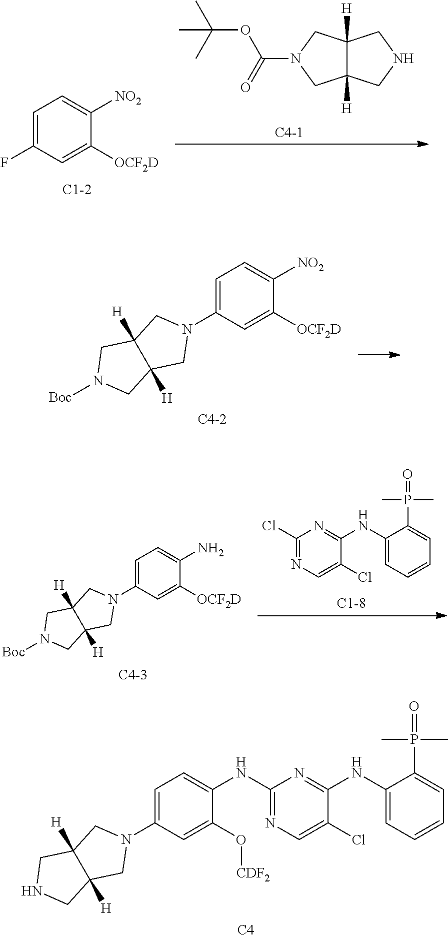

(1) Synthesis of C4-2

The Compound C1-2 (700 mg, 3.37 mmol) and Compound C4-1 (928 mg, 4.38 mmol) were dissolved in DMF (15 ml), and anhydrous potassium carbonate (1.02 g, 7.40 mmol) was added, and then stirred overnight at 80° C. The solvent DMF was removed by evaporation under reduced pressure, the residue was diluted with water, extracted with ethyl acetate, and the combined organic phases were washed with saturated salt water, dried over anhydrous sodium sulfate, concentrated to obtain crude product which was purified by column chromatography to obtain 990 mg of Compound C4-2.

¹H NMR (400 MHz, CDCl₃) δ 8.06-8.04 (d, 1H, J=9.29 Hz), 6.39-636 (dd, 1H, J=9.32 Hz, 2.64 Hz), 6.32-6.31 (d, 1H, J=2.50 Hz), 3.67 (br, 4H), 3.38-3.27 (m, 4H), 3.07 (br, 2H), 1.46 (s, 9H).

(2) Synthesis of C4-3

The Compound C4-2 (130 mg, 1.15 mmol) was dissolved in methanol (10 ml), then Pd (OH)₂/C (26 mg) was added, then stirred under hydrogen atmosphere for 4h. TLC detection showed the reaction was completed. The reaction solution was filtered through diatomite, washed with methanol, and the filterate was concentrated to obtain 115 mg Compound C4-3. Ms [M+H] 371.2.

(3) Synthesis of Compound C4

The Compound C4-3 (138 mg, 0.37 mmol) and Compound C1-8 (124 mg, 0.392 mmol) were dissolved in ethylene glycol monomethyl ether (10 ml), then 4M HCl/dioxane (0.28 ml, 1.12 mmol) was added, stirred overnight at 120° C., concentrated under reduced pressure, alkalized with saturated sodium bicarbonate solution, extracted with ethyl acetate. The combined organic phases were washed with saturated salt water, dried over anhydrous sodium sulfate, and concentrated to obtain crude product which was purified by Prep-TLC to obtain 20 mg Compound C4. Ms [M+H] 550.2.

¹H NMR (400 MHz, CD₃OD) δ 8.23-8.20 (q, 1H), 8.07 (s, 1H), 7.91 (s, 1H), 7.50-7.40 (m, 2H), 7.34-7.30 (t, 1H), 7.14-7.09 (m, 1H), 6.60-6.48 (m, 2H), 3.52-3.50 (m, 2H), 3.40-3.35 (m, 2H), 3.21-3.11 (m, 6H), 1.76-1.72 (d, 6H, J=13.73 Hz).

### Example 5 Synthesis of Compound C5

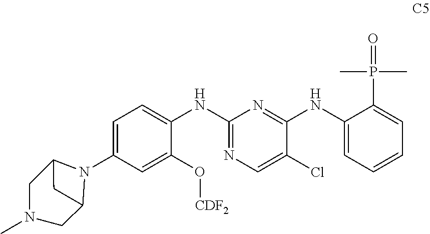

The experimental process was as follows:

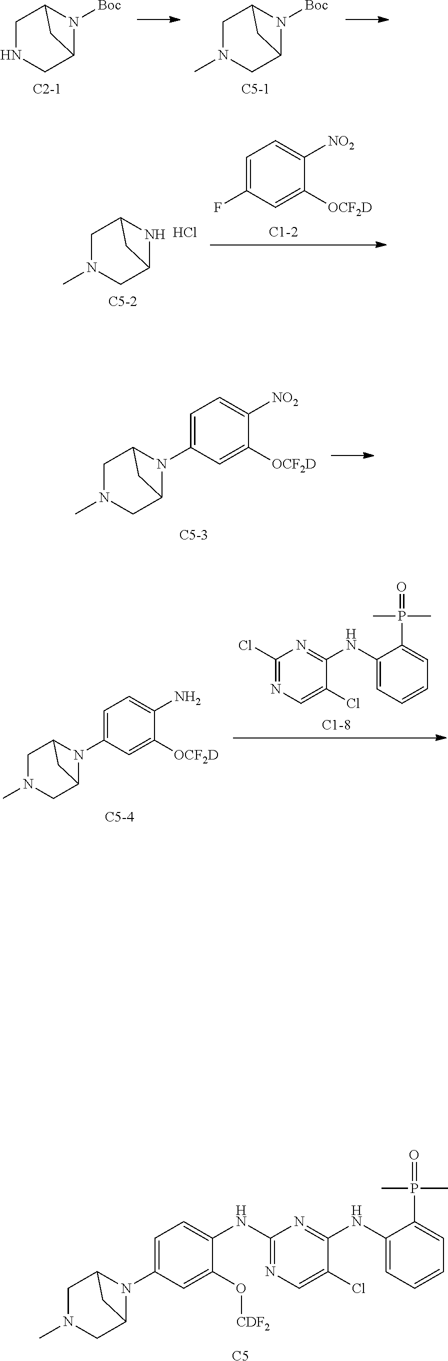

(1) Synthesis of C5-1

The Compound C2-1 (300 mg, 1.51 mmol) and 37% aqueous HCHO solution (492 mg, 6.04 mmol) were dissolved in THF (15 ml), and HOAc (363 mg, 6.04 mmol) was added, then stirred for 1 h at r.t, NaBH (OAc)₃ (1.28 g, 6.04 mmol) was added, and stirred overnight at room temperature. The solvent THF was removed by evaporation under reduced pressure, and pH was adjusted to >7 with saturated NaHCO₃, extracted with ethyl acetate, washed the combined organic phases with saturated salt water, dried over anhydrous sodium sulfate, and concentrated to obtain 274 mg of crude Compound C5-1.

¹H NMR (400 MHz, CD₃OD) δ 4.04 (s, 2H), 3.06-2.94 (d, 2H, J=8.95 Hz), 2.81-2.78 (d, 2H, J=10.64 Hz), 2.39-2.34 (m, 4H), 1.72-1.70 (d, 2H, J=7.76 Hz), 1.45 (s, 9H).

(2) Synthesis of C5-2

The Compound C5-1 (274 mg, 1.29 mmol) was dissolved in 0.5 ml dioxane, 4M HCl/dioxane (3 ml, 12 mmol) was added, then stirred for 3h at r.t, ether was added, stirred for 10 min, then filtered, and the filter cake was evaporated to dryness to obtain 244 mg of Compound C5-2 which was directly used for the next step.

(3) Synthesis of C5-3

The Compound C5-2 (223 mg, 1.51 mmol) and Compound C1-2 (241.1 mg, 1.16 mmol) were dissolved in 15 ml MeCN, then K₂CO₃ (799.7 mg, 5.79 mmol) was added, stirred overnight at 80° C., concentrated under reduced pressure. Water was added, extracted with ethyl acetate. The combined organic phases were washed with saturated salt water, dried over anhydrous sodium sulfate, and purified by column chromatography to obtain 134 mg of Compound C5-3.

¹H NMR (400 MHz, CDCl₃) δ 8.01-7.99 (d, 1H, J=9.08 Hz), 6.28-6.25 (dd, 1H, J=11.44 Hz), 6.20-6.19 (t, 1H), 4.36-4.34 (d, 2H, J=5.72 Hz), 2.99-2.97 (d, 2H, J=11.2 Hz), 2.93-2.90 (d, 2H, J=11.2 Hz), 2.65-2.60 (m, 1H), 2.26 (s, 1H), 2.17-2.15 (d, 1H, J=7.88 Hz).

(4) Synthesis of C5-4

The Compound C5-3 (134 mg, 0.45 mmol) was dissolved in MeOH (4 ml) and EA (4 ml), then Pd/C (13.4 mg) was added, stirred for 2h at r.t, filtered, and the filtrate was evaporated to dryness to obtain 110 mg of Compound C5-4.

¹H NMR (400 MHz, CD₃OD) δ 6.85-6.83 (d, 1H, J=9.12 Hz), 6.30-6.29 (m, 2H), 4.20-4.19 (d, 2H, J=5.4 Hz), 3.12-3.09 (d, 2H, J=11.6 Hz), 2.89-2.86 (d, 2H, J=11.68 Hz), 2.55-2.50 (m, 1H), 2.18 (s, 1H), 2.10-2.08 (d, 1H, J=8.12 Hz).

(5) Synthesis of Compound C5

The Compound C5-4 (103 mg, 0.38 mmol) and Compound C1-8 (126.6 mg, 0.4 mmol) were dissolved in ethylene glycol monomethyl ether (10 ml), then 4M HCl/dioxane (0.29 ml, 1.14 mmol) was added, stirred overnight at 120° C., concentrated under reduced pressure. The pH was adjusted to >7 with saturated sodium bicarbonate solution, extracted with ethyl acetate. The combined organic phases were washed with saturated salt water, dried over anhydrous sodium sulfate, and purified by Prep-TLC to obtain 134 mg Compound C5.

¹H NMR (400 MHz, CD₃OD) δ 8.33-8.30 (m, 1H), 8.01 (s, 1H), 7.98 (s, 1H), 7.59-7.53 (m, 1H), 7.42-7.38 (t, 1H), 7.36-7.34 (d, 1H, J=8.92 Hz), 7.22-7.20 (m, 1H), 6.55-6.52 (t, 2H), 4.45-4.43 (m, 1H), 3.91-3.89 (m, 1H), 2.80-2.69 (br, 3H), 2.35 (s, 3H), 2.29 (br, 1H), 2.06 (br, 2H), 1.87 (s, 3H), 1.84 (s, 3H).

### Example 6 Synthesis of Compound C6

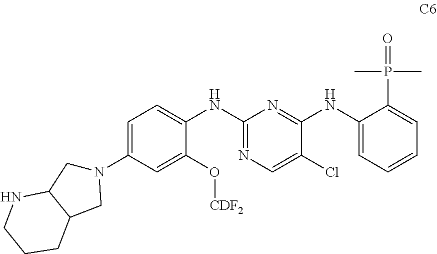

The experimental process was as follows:

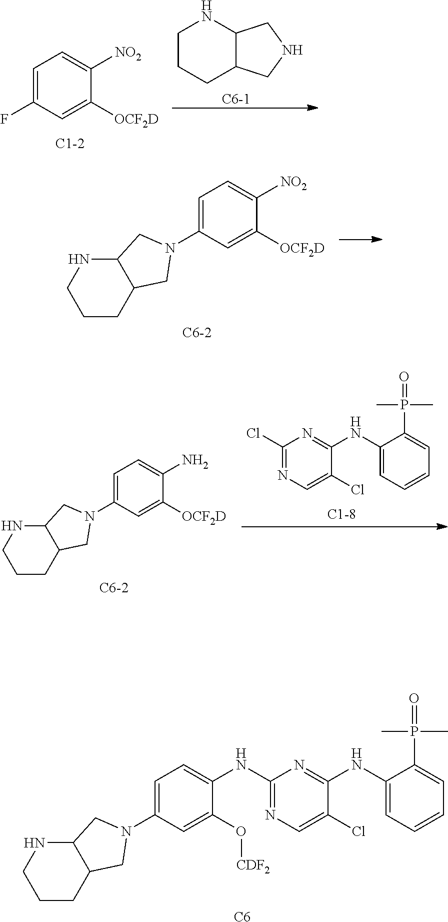

(1) Synthesis of C6-2

The Compound C1-2 (100 mg, 0.48 mmol) and Compound C6-1 (79 mg, 0.63 mmol) were dissolved in DMF (3 ml), and potassium carbonate (146 mg, 1.06 mmol) was added, and then stirred overnight at 85° C. The solvent DMF was removed by evaporation under reduced pressure, the residue was diluted with water, extracted with ethyl acetate, and the combined organic phases were washed with saturated salt water, dried over anhydrous sodium sulfate, concentrated to obtain crude product which was purified by column chromatography to obtain 180 mg of Compound C6-2.

¹H NMR (400 MHz, CDCl3) δ 8.04-8.02 (d, 1H, J=9.43 Hz), 6.36-6.33 (m, 1H), 6.29-6.28 (m, 1H), 3.58-3.52 (m, 3H), 3.41-3.36 (t, 1H), 3.31-3.27 (m, 1H), 3.06-3.02 (m, 1H), 2.74-2.67 (m, 1H), 2.49-2.43 (m, 1H), 2.21 (br, 1H), 1.83-1.54 (m, 4H).

(2) Synthesis of C6-3

The Compound C6-2 (180 mg, 0.57 mmol) was dissolved in methanol (10 ml), and palladium hydroxide (18 mg) was added and stirred for 6h at room temperature under hydrogen atmosphere. The reaction solution was filtered through diatomite, washed with methanol, and the filtrate was concentrated to obtain 134 mg Compound C6-3. MS[M+H] 285.

(3) Synthesis of Compound C6

The Compound C6-2 (108 mg, 0.38 mmol) and Compound C1-8 (126 mg, 0.40 mmol) were dissolved in ethylene glycol monomethyl ether (5 ml), then 4M HCl/dioxane (0.29 ml, 1.14 mmol) was added, stirred overnight at 120° C., concentrated under reduced pressure, alkalized with saturated sodium bicarbonate solution, extracted with ethyl acetate. The combined organic phases were washed with saturated salt water, dried over anhydrous sodium sulfate, and concentrated to obtain crude product. Certain amount of the crude compound was purified by Prep-TLC to obtain 18 mg Compound C6. Ms [M+H] 564.

¹H NMR (400 MHz, CD₃OD) δ, 8.38-8.35 (q, 1H), 8.02 (s, 1H), 7.61-7.56 (m, 1H), 7.52-7.50 (d, 1H, J=8.57 Hz), 7.46-742 (t, 1H), 7.25-7.21 (m, 1H), 6.50-6.46 (m, 2H), 3.94-3.92 (t, 1H), 3.68-3.64 (m, 1H), 3.55-3.45 (m, 3H), 3.37 (s, 2H), 3.31-3.30 (m, 1H), 3.09-3.03 (m, 1H), 2.88-2.84 (m, 1H), 1.99-1.95 (m, 2H), 1.87-1.84 (d, 6h, J=13.25 Hz).

### Example 7 Synthesis of Compounds C7 and C8

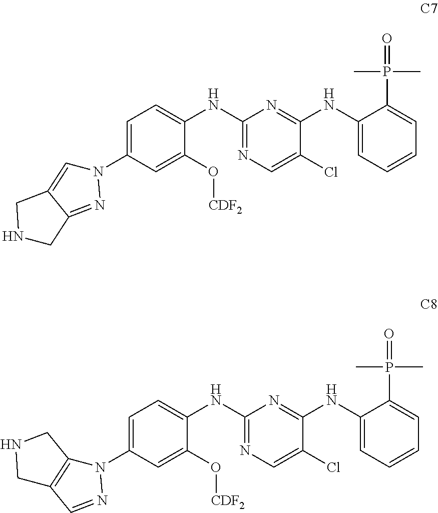

The experimental process was as follows:

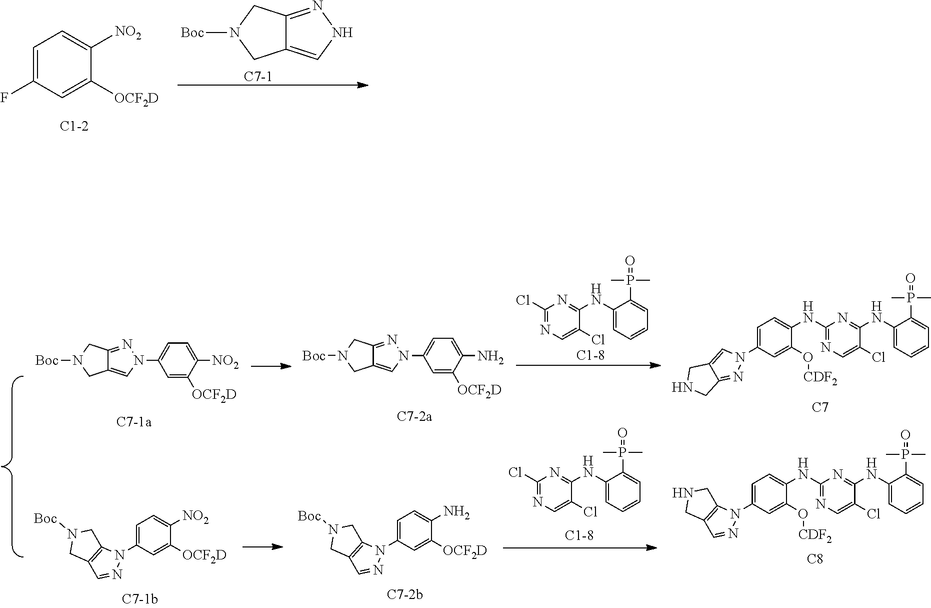

(1) Synthesis of Compounds C7-1a and C7-1b

The Compound C1-2 (500 mg, 2.40 mmol) and Compound C7-1 (654 mg, 3.13 mmol) were dissolved in DMF (10 ml), and potassium carbonate (730 mg, 5.29 mmol) was added, and then stirred overnight at 85° C. The solvent DMF was removed by evaporation under reduced pressure, the residue was diluted with water, extracted with ethyl acetate, and the combined organic phases were washed with saturated salt water, dried over anhydrous sodium sulfate, concentrated to obtain crude product which was purified by column chromatography to obtain 336 mg of Compound C7-1a and 172 mg of Compound C7-1b.

Compound C7-1a: ¹H NMR (400 MHz, CDCl₃) δ 8.11-8.09 (d, 1H, J=10.08 Hz), 7.76-7.71 (m, 2H), 7.64-7.61 (m, 1H), 4.58-4.50 (m, 4H), 1.53 (s, 9H).

Compound C7-1b: ¹H NMR (400 MHz, CDCl₃) δ 8.13-8.09 (m, 1H), 7.70 (s, 1H), 7.59-7.55 (d, 1H, J=17.70 Hz), 7.49-7.46 (m, 1H), 4.85-4.79 (m, 2H), 4.53-4.46 (m, 2H), 1.53 (s, 9H).

(2) Synthesis of C7-2a

The Compound C7-1a (140 mg, 0.35 mmol) was dissolved in methanol (5 ml), Pd/C (14 mg) was added, and stirred at room temperature under hydrogen atmosphere for 4h. The reaction solution was filtered through diatomite, washed with methanol, and the filtrate was concentrated to obtain 136 mg Compound C7-2a. MS[M+H] 368.

(3) Synthesis of Compound C7

The Compound C7-2a (104 mg, 0.28 mmol) and Compound C1-8 (94 mg, 0.30 mmol) were dissolved in ethylene glycol monomethyl ether (3 ml), then 4M HCl/dioxane (0.22 ml, 0.85 mmol) was added, stirred overnight at 120° C., concentrated under reduced pressure, alkalized with saturated sodium bicarbonate solution, extracted with ethyl acetate. The combined organic phases were washed with saturated salt water, dried over anhydrous sodium sulfate, and concentrated to obtain crude product which was purified by Prep-TLC to obtain 38 mg Compound C7. Ms [M+H] 547.2.

¹H NMR (400 MHz, CD₃OD) δ, 8.31-8.28 (q, 1H), 8.16-8.13 (m, 3H), 7.70-7.64 (m, 2H), 7.60-7.56 (m, 1H), 7.50-7.47 (m, 1H), 7.34-7.30 (m, 1H), 4.51-4.49 (d, 4H, J=6.37 Hz), 1.88-1.85 (d, 6H, J=12.53 Hz).

(4) Synthesis of C7-2b

The Compound C7-1b (70 mg, 0.18 mmol) was dissolved in methanol (5 ml), Pd/C (7 mg) was added, and stirred at room temperature under hydrogen atmosphere for 4h. The reaction solution was filtered through diatomite, washed with methanol, and the filtrate was concentrated to obtain 52 mg Compound C7-2b. MS[M+H] 368.

(5) Synthesis of Compound C8

The Compound C7-2b (52 mg, 0.14 mmol) and Compound C1-8 (47 mg, 0.15 mmol) were dissolved in ethylene glycol monomethyl ether (2 ml), then 4M HCl/dioxane (0.11 ml, 0.43 mmol) was added, stirred overnight at 120° C., concentrated under reduced pressure, alkalized with saturated sodium bicarbonate solution, extracted with ethyl acetate. The combined organic phases were washed with saturated salt water, dried over anhydrous sodium sulfate, and concentrated to obtain crude product which was purified by Prep-HPLC to obtain 4 mg Compound C8. Ms [M+H] 547.2.

¹H NMR (400 MHz, CD₃OD) δ 8.61-8.58 (m, 1H), 7.85 (s, 1H), 7.46-7.40 (m, 2H), 7.34 (s, 1H), 7.20-7.17 (m, 1H), 7.10-7.06 (m, 2H), 6.83-6.81 (d, 1H, J=8.43 Hz), 4.68-4.63 (m, 2H), 4.44 (br, 2H), 1.77-1.74 (d, 6H, J=14.13 Hz).

### Example 8 Synthesis of Compounds C9 and C10

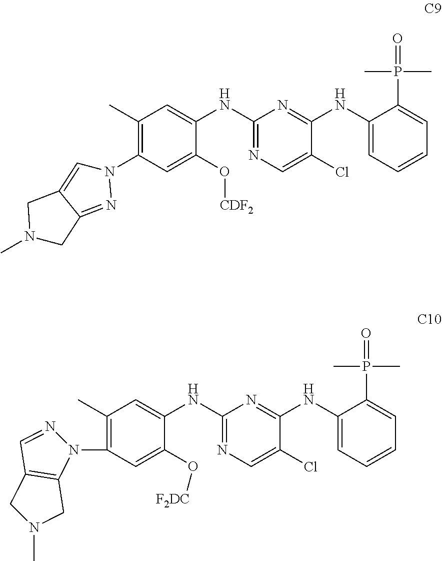

The experimental process was as follows:

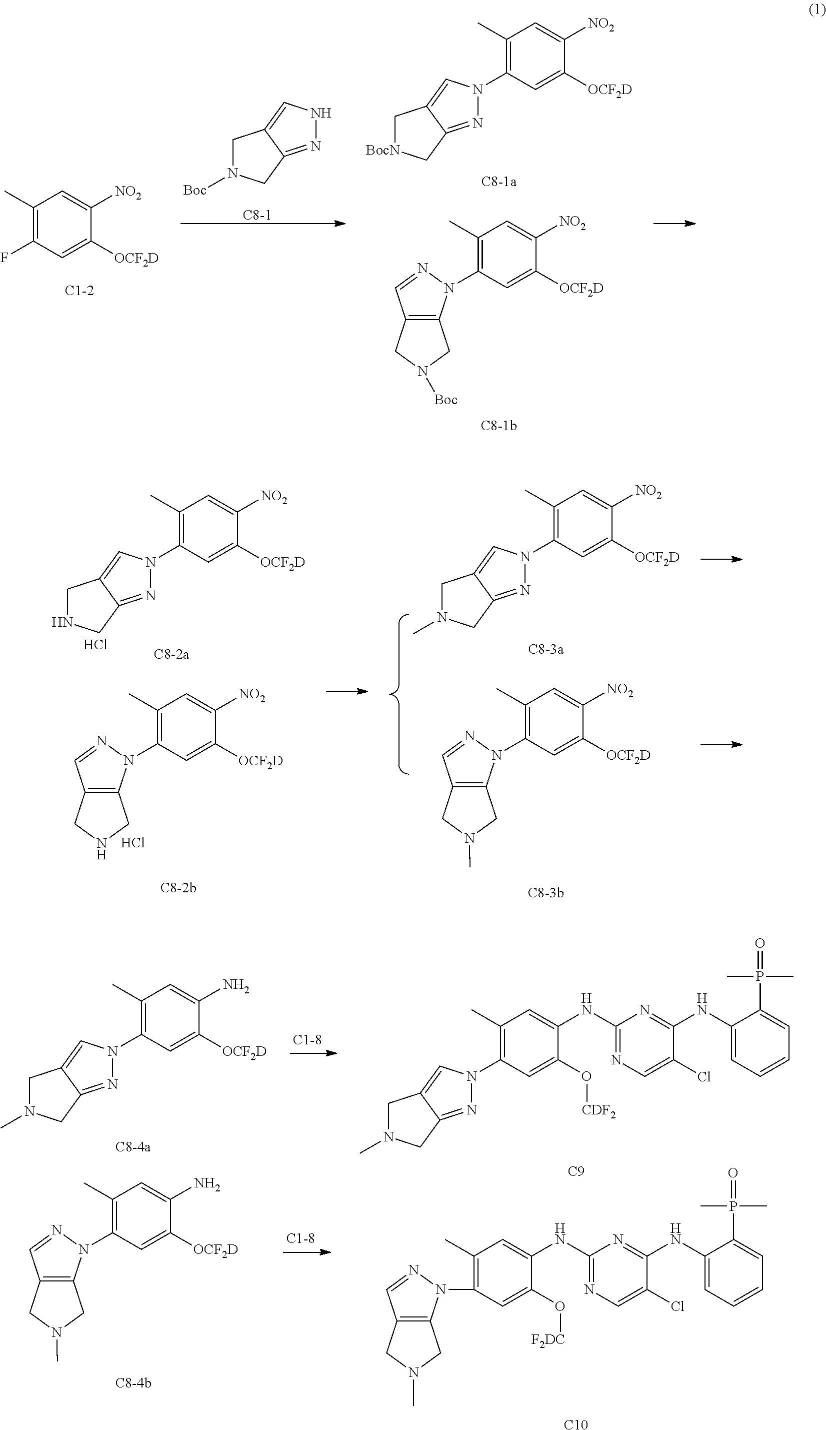

Synthesis of C8-1a and C8-1b

The Compound C1-2 (500 mg, 2.25 mmol) and Compound C8-1 (612 mg, 2.96 mmol) were dissolved in DMF (20 ml), and potassium carbonate (932.4 mg, 6.76 mmol) was added, and then stirred at 80° C. for 4h. The solvent DMF was removed by evaporation under reduced pressure, the residue was diluted with water, extracted with ethyl acetate, and the combined organic phases were washed with saturated salt water, dried over anhydrous sodium sulfate, concentrated to obtain crude product which was purified by column chromatography to obtain 531 mg of mixture of Compound C8-1a and Compound C8-1b. Ms [M+H] 412.2.

(2) Synthesis of C8-2a and C8-2b

The mixture (531 mg, 1.34 mmol) of Compounds C8-1a and C8-1b was dissolved in 4M HCl/dioxane and stirred at room temperature for 2h. The reaction solution was then concentrated under reduced pressure to obtain 471 mg of crude Compounds C8-2a and C8-2b which was directly used for the next step.

(3) Synthesis of C8-3a and C₈₋₃b

The Compounds C8-2a and C8-2b (471 mg, 1.51 mmol) were dissolved in THF, then 37% aqueous formaldehyde solution (491.1 mg, 6.06 mmol) and glacial acetic acid (363.5 mg, 6.06 mmol) were added, stirred at room temperature for 1 h, then sodium triacetoxyborohydride (1.6 g, 7.57 mmol) was added in batches and stirred overnight at room temperature. The reaction solution was concentrated, alkalized to pH >7 with saturated sodium bicarbonate solution, extracted with dichloromethane, washed with saturated salt water, dried over anhydrous sodium sulfate, and purified by column chromatography to obtain 101 mg of Compound C8-3a and 256 mg of Compound C8-3b.

Compound C8-3a ¹H NMR (400 MHz, CDCl3) δ 7.93 (s, 1H), 7.48 (s, 1H), 7.25 (s, 1H), 3.86-3.84 (m, 4H), 2.65 (s, 3H), 2.43 (s, 3H).

Compound C8-3b ¹H NMR (400 MHz, CDCl₃) δ 7.94 (s, 1H), 7.43 (s, 1H), 7.36 (s, 1H), 3.85 (s, 2H), 3.81 (s, 2H), 2.67 (s, 3H), 2.45 (s, 3H).

(4) Synthesis of C8-4a

The Compound C8-3a (70 mg, 0.22 mmol) was dissolved in methanol (5 ml), Pd/C (7 mg) was added, then stirred overnight under hydrogen atmosphere. The filtrate was filtered through diatomite, washed with methanol, and concentrated to obtain 44.7 mg of Compound C8-4a.

¹H NMR (400 MHz, CDCl₃) δ 7.36 (s, 1H), 6.96 (s, 1H), 6.66 (s, 2H), 3.97 (br, s, 2H), 3.86 (s, 2H). 3.76 (s, 2H). 2.64 (s, 3H). 2.09 (s, 3H).

(5) Synthesis of Compound C9

The Compound C8-4a (44.7 mg, 0.15 mmol) and Compound C1-8 (62.25 mg, 0.20 mmol) were dissolved in ethylene glycol monomethyl ether (2 ml), then 4M HCl/dioxane (0.06 ml, 0.23 mmol) was added, stirred overnight at 100° C., concentrated under reduced pressure, alkalized with saturated sodium bicarbonate solution, extracted with ethyl acetate. The combined organic phases were washed with saturated salt water, dried over anhydrous sodium sulfate, and concentrated to obtain crude product which was purified by Prep-TLC to obtain 19 mg Compound C9. Ms [M+H] 576.1.

¹H NMR (400 MHz, CD₃OD) δ 8.18-8.15 (m, 1H), 8.07 (s, 1H), 8.01 (s, 1H), 7.59-7.54 (m, 1H), 7.51-7.47 (m, 1H), 7.35 (s, 1H), 7.23-7.18 (m, 1H), 7.05 (s, 1H), 3.80 (s, 2H), 3.72 (s, 2H), 2.55 (s, 3H), 1.91 (s, 3H), 1.77 (s, 3H), 1.74 (s, 3H).

(6) Synthesis of C8-4b

The Compound C8-3b (92 mg, 0.28 mmol) was dissolved in methanol (5 ml), Pd/C (9 mg) was added, then stirred for 2h under hydrogen atmosphere. The filtrate was filtered through diatomite, washed with methanol, and concentrated to obtain 70 mg of Compound C8-4b.

¹H NMR (400 MHz, CDCl₃) δ 7.14 (s, 1H), 7.01 (s, 1H), 6.65 (s, 2H), 3.94 (br, s, 2H), 3.83 (s, 2H). 3.80 (s, 2H). 2.65 (s, 3H). 2.09 (s, 3H).

(7) Synthesis of Compound C10

The Compound C8-4b (83 mg, 0.28 mmol) and Compound C1-8 (97.8 mg, 0.31 mmol) were dissolved in ethylene glycol monomethyl ether (2 ml), then 4M HCl/dioxane (0.18 ml, 0.7 mmol) was added, stirred overnight at 100° C., concentrated under reduced pressure, alkalized with saturated sodium bicarbonate solution, extracted with ethyl acetate. The combined organic phases were washed with saturated salt water, dried over anhydrous sodium sulfate, and concentrated to obtain crude product which was purified by Prep-TLC to obtain 6.8 mg Compound C10. Ms [M+H] 576.1.

¹H NMR (400 MHz, CD₃OD) δ 8.18-8.15 (m, 1H), 8.07 (s, 1H), 7.98 (s, 1H), 7.59-7.54 (m, 1H), 7.51-7.47 (m, 1H), 7.44 (s, 1H), 7.22-7.18 (m, 1H), 7.08 (s, 1H), 3.82 (s, 4H), 2.62 (s, 3H), 1.94 (s, 3H), 1.77 (s, 3H), 1.74 (s, 3H).

### Example 9 Synthesis of Compound C11

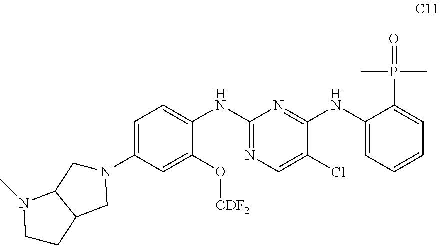

The experimental process was as follows:

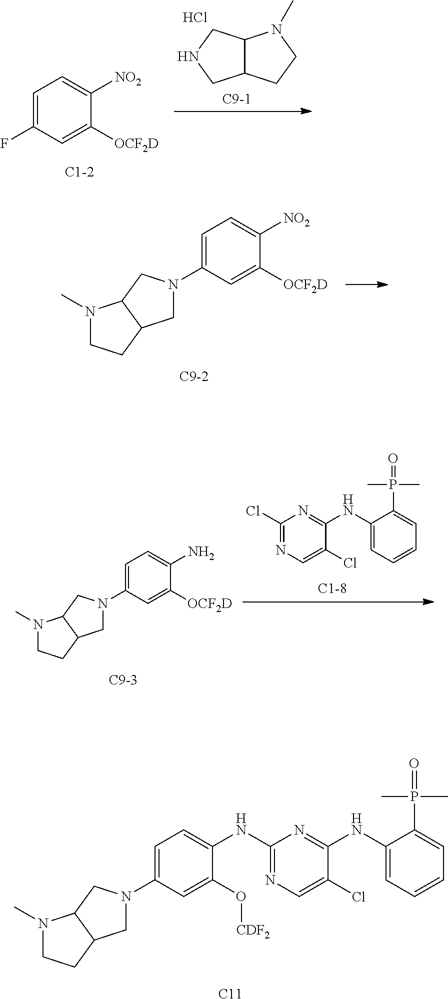

(1) Synthesis of C9-2

The Compound C1-2 (107 mg, 0.52 mmol) and Compound C9-1 (109 mg, 0.67 mmol) were dissolved in DMF (2 ml), and potassium carbonate (227 mg, 1.65 mmol) was added, and then stirred overnight at 85° C. The solvent DMF was removed by evaporation under reduced pressure, the residue was diluted with water, extracted with ethyl acetate, and the combined organic phases were washed with saturated salt water, dried over anhydrous sodium sulfate, concentrated to obtain crude product which was purified by column chromatography to obtain 91 mg of Compound C9-2.

¹H NMR (400 MHz, CDCl₃) δ 8.04-8.02 (d, 1H, J=9.55 Hz), 6.39-636 (dd, 1H, J=9.10 Hz, 2.39 Hz), 6.32-6.31 (d, 1H, J=2.50 Hz), 3.66-3.61 (m, 1H), 3.52-3.49 (m, 1H), 3.43-3.40 (m, 1H), 3.31-3.27 (m, 1H), 3.23-3.18 (m, 1H), 3.00-3.93 (m, 2H), 2.43-2.36 (m, 4H), 2.20-2.12 (m, 1H), 1.84-1.75 (m, 1H).

(2) Synthesis of C9-3

The Compound C9-2 (91 mg, 0.29 mmol) was dissolved in methanol (10 ml), then Pd (OH) 2 (10 mg) was added, and stirred under hydrogen atmosphere for 4h. TLC detection showed the reaction was completed. The reaction solution was filtered through diatomite, washed with methanol, and the filtrate was concentrated to obtain 41 mg Compound C9-3. Ms [M+H] 285.2.

(3) Synthesis of Compound C11

The Compound C9-3 (41 mg, 0.14 mmol) and Compound C1-8 (48 mg, 0.15 mmol) were dissolved in ethylene glycol monomethyl ether (5 ml), then 4M HCl/dioxane (0.11 ml, 0.43 mmol) was added, stirred overnight at 120° C., concentrated under reduced pressure, alkalized with saturated sodium bicarbonate solution, extracted with ethyl acetate. The combined organic phases were washed with saturated salt water, dried over anhydrous sodium sulfate, and concentrated to obtain crude product. Certain amount of the crude compound was purified by Prep-TLC to obtain 47 mg Compound C11. Ms [M+H] 564.2.

¹H NMR (400 MHz, CDCl₃) δ 10.89 (s, 1H), 8.59-8.56 (q, 1H), 8.07 (s, 1H), 7.93-7.91 (d, 1H, J=9.05 Hz), 7.46-7.42 (t, 1H), 7.30-7.24 (m, 1H), 7.13-7.08 (m, 1H), 6.88 (s, 1H), 6.51-6.46 (m, 2H), 3.61-3.58 (m, 1H), 3.32-3.15 (m, 5H), 3.02 (br, 1H), 2.64-2.45 (m, 4H), 2.23 (br, 1H), 1.85-1.82 (d, 6H, J=13.78 Hz).

### Example 10. Synthesis of Compound C12

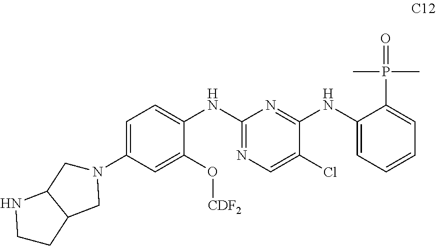

The experimental process was as follows:

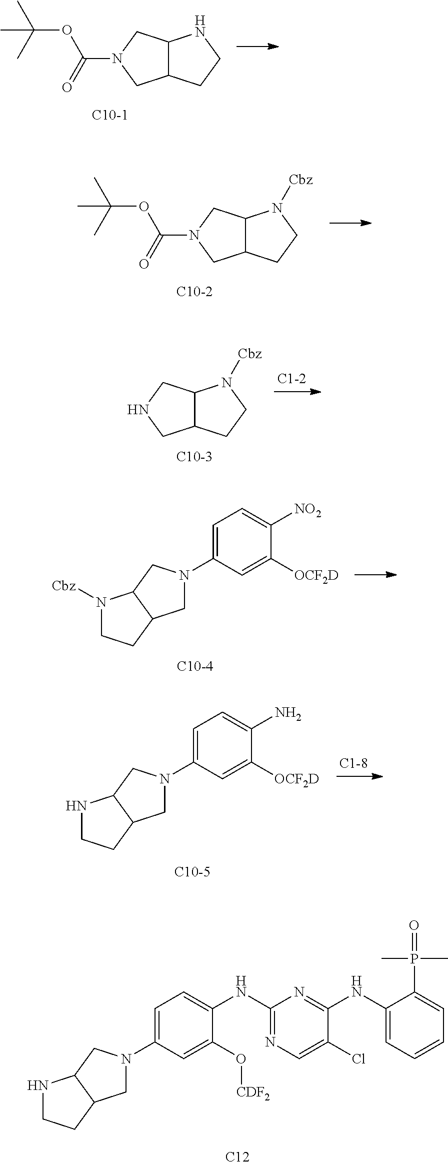

(1) Synthesis of C10-2

The Compound C10-1 (100 mg, 0.47 mmol) and benzyl chloroformate (0.07 ml, 0.51 mmol) were dissolved in dichloromethane (5 ml), then triethylamine (0.07 ml, 0.50 mmol) was added and stirred overnight at room temperature. The reaction solution was diluted with dichloromethane, washed with saturated salt water, dried over anhydrous sodium sulfate, concentrated to obtain a crude product which was purified by column chromatography to obtain 158 mg of Compound C10-2.

¹H NMR (400 MHz, CDCl₃) δ 7.37-7.30 (m, 5H), 5.18-5.09 (m, 2H), 4.33-4.26 (m, 1H), 3.61-3.47 (m, 5H), 3.29-3.15 (m, 1H), 2.95-2.85 (m, 1H), 2.05-1.96 (m, 1H), 1.82-1.73 (m, 1H), 1.45 (m, 9H).

(2) Synthesis of C10-3

The Compound C10-2 (158 mg, 0.70 mmol) was dissolved in trifluoroacetic acid (5 ml), stirred at room temperature for 4 hours, and concentrated under reduced pressure. The residue was diluted with saturated sodium bicarbonate solution and then extracted with dichloromethane. The combined organic phases were washed with saturated salt water, dried over anhydrous sodium sulfate, and concentrated to obtain 62 mg crude product, which was directly used for the next step.

¹H NMR (400 MHz, CDCl₃) δ 7.32-7.21 (m, 5H), 5.05 (s, 2H), 4.43-4.33 (m, 1H), 3.60-3.55 (m, 1H), 3.47-3.36 (m, 4H), 3.08-3.05 (m, 2H), 2.07-2.05 (m, 1H), 1.79 (br, 1H).

(3) Synthesis of C10-4

The Compound C1-2 (40 mg, 0.19 mmol) and Compound C10-3 (62 mg, 0.25 mmol) were dissolved in DMF (2 ml), and potassium carbonate (59 mg, 0.43 mmol) was added, and then stirred overnight at 85° C. The solvent DMF was removed by evaporation under reduced pressure, the residue was diluted with water, extracted with ethyl acetate, and the combined organic phases were washed with saturated salt water, dried over anhydrous sodium sulfate, concentrated to obtain crude product which was purified by column chromatography to obtain 87 mg of Compound C10-4.

¹H NMR (400 MHz, CDCl₃) δ 8.04-8.02 (d, 1H, J=9.43 Hz), 7.39-7.30 (m, 5H), 6.39-6.20 (m, 2H), 5.22-5.10 (m, 2H), 4.51-4.45 (m, 1H), 3.72-3.49 (m, 5H), 3.26-3.14 (m, 2H), 2.16-2.11 (m, 1H), 1.94-1.85 (m, 1H).

(4) Synthesis of C10-5

The Compound C10-4 (45 mg, 0.10 mmol) was dissolved in methanol (10 ml), then palladium hydroxide (10 mg) was added and stirred at room temperature under hydrogen atmosphere for 2h. The reaction solution was filtered through diatomite, washed with methanol, and the filtrate was concentrated to obtain 28 mg Compound C10-5. MS[M+H] 271.

(5) Synthesis of Compound C12

The Compound C10-5 (28 mg, 0.11 mmol) and Compound C1-8 (43 mg, 0.14 mmol) were dissolved in ethylene glycol monomethyl ether (2 ml), then 4M HCl/dioxane (0.078 ml, 0.31 mmol) was added, stirred overnight at 120° C., concentrated under reduced pressure, alkalized with saturated sodium bicarbonate solution, extracted with ethyl acetate. The combined organic phases were washed with saturated salt water, dried over anhydrous sodium sulfate, and concentrated to obtain crude product. Certain amount of the crude compound was purified by Prep-TLC to obtain 15 mg Compound C12. Ms [M+H] 550.

¹H NMR (400 MHz, CD₃OD) δ, 8.23-8.20 (q, 1H), 7.92 (s, 1H), 7.51-7.45 (m, 2H), 7.35-7.31 (t, 1H), 7.14-7.10 (m, 1H), 6.52-6.49 (m, 2H), 4.30-4.27 (t, 1H), 3.73-3.70 (m, 2H), 3.37-3.17 (m, 1H), 2.29-2.24 (m, 1H), 1.98-1.94 (m, 1H), 1.75-1.72 (d, 6h, J=13.29 Hz).

### Example 11. Synthesis of Compound C13

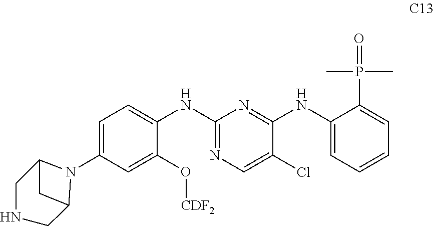

The experimental process was as follows:

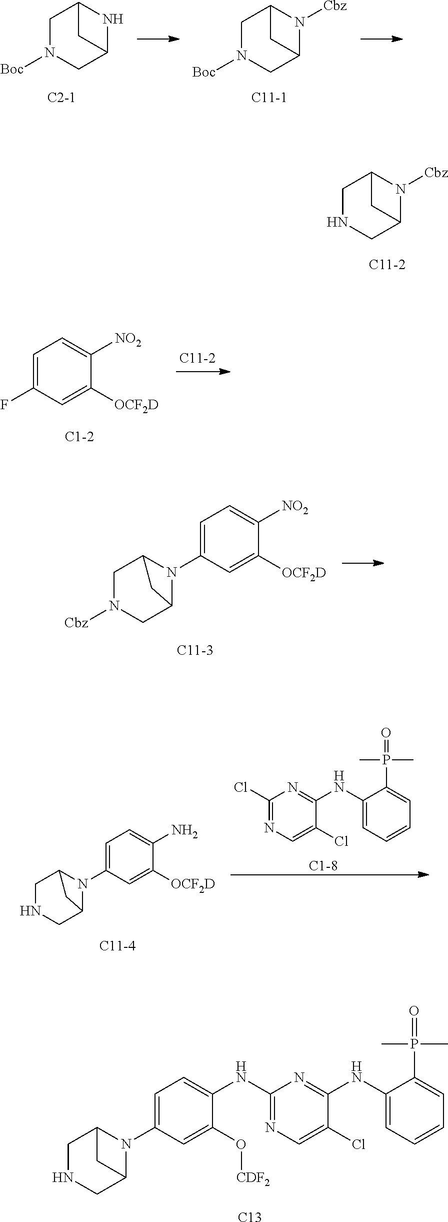

(1) Synthesis of C11-1

The Compound C2-1 (300 mg, 1.52 mmol) and benzyl chloroformate (291 mg, 1.71 mmol) were dissolved in dichloromethane (20 ml), and triethylamine (174 mg, 1.71 mmol) was added. The reaction was stirred overnight at room temperature. The reaction solution was diluted with dichloromethane, washed with saturated salt water, dried over anhydrous sodium sulfate and purified by concentrated column chromatography to obtain 447 mg of Compound C11-1.

(2) Synthesis of C11-2

The Compound C11-1 (447 mg, 1.35 mmol) was dissolved in trifluoroacetic acid (10 ml), stirred at room temperature for 4 hours, and concentrated under reduced pressure. The residue was diluted with saturated sodium bicarbonate solution and then extracted with dichloromethane. The combined organic phases were washed with saturated salt water, dried over anhydrous sodium sulfate, and concentrated to obtain 292 mg crude product, which was directly used for the next step.

(3) Synthesis of C11-3

The Compound C1-2 (201 mg, 0.97 mmol) and Compound C11-2 (292 mg, 1.26 mmol) were dissolved in DMF (10 ml), and potassium carbonate (293 mg, 2.13 mmol) was added, and then stirred overnight at 85° C. The solvent DMF was removed by evaporation under reduced pressure, the residue was diluted with water, extracted with ethyl acetate, and the combined organic phases were washed with saturated salt water, dried over anhydrous sodium sulfate, concentrated to obtain crude product which was purified by column chromatography to obtain 406 mg of Compound C11-3.

(4) Synthesis of C11-4

The Compound C11-3 (406 mg, 0.97 mmol) was dissolved in methanol (20 ml), and palladium hydroxide (41 mg) was added and stirred for 16h at room temperature under hydrogen atmosphere. The reaction solution was filtered by diatomite and washed with methanol. The filtrate was concentrated to obtain a crude product, which was purified by Prep-TLC to obtain 191 mg of Compound C11-4. MS [M+H] 256.2.

(5) Synthesis of Compound C13

The Compound C11-4 (191 mg, 0.75 mmol) and Compound C1-8 (248 mg, 0.78 mmol) were dissolved in ethylene glycol monomethyl ether (3 ml), methanesulfonic acid (0.12 ml, 1.86 mmol) was added, and stirred overnight at 90° C. under nitrogen protection. The reaction solution was concentrated under reduced pressure, and pH was adjusted to >7 with saturated sodium bicarbonate solution, extracted with ethyl acetate. The combined organic phases were washed with saturated salt water, dried over anhydrous sodium sulfate, and purified by Prep-HPLC to obtain 30 mg of Compound C13.

¹H NMR (400 MHz, CDCl₃) δ 10.85 (s, 1H), 8.55-8.52 (q, 1H), 8.06 (s, 1H), 7.83-7.81 (d, 1H, J=9.30 Hz), 7.43-7.39 (t, 1H), 7.29-7.24 (m, 1H), 7.12-7.08 (m, 1H), 6.92 (s, 1H), 6.47-6.43 (m, 2H), 4.29 (br, 1H), 3.77-3.74 (m, 1H), 3.19-3.16 (m, 2H), 2.97-2.92 (m, 1H), 2.72-2.67 (m, 1H), 2.20-2.08 (m, 2H), 1.85-1.81 (d, 6H, J=12.79 Hz).

### Example 12. Synthesis of Compound C14

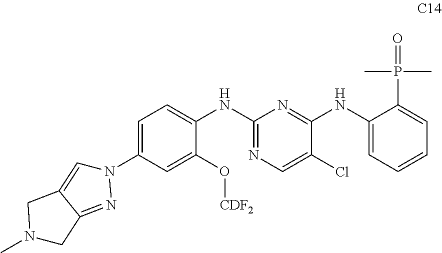

The experimental process was as follows:

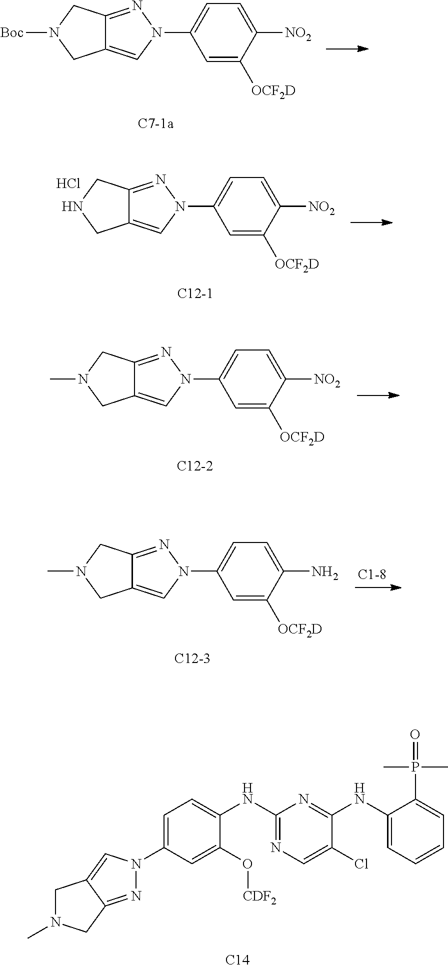

(1) Synthesis of C12-1

The Compound C7-1a (200 mg, 0.55 mmol) was dissolved in 4M HCl/dioxane (10 ml), stirred at room temperature for 4 hours, and concentrated under reduced pressure to obtain 187 mg crude Compound C12-1 which was directly used for the next step without purification.

(2) Synthesis of C12-2

The crude Compound C12-1 (187 mg, 0.55 mmol) was dissolved in tetrahydrofuran (10 ml), then 37% aqueous formaldehyde solution (177 mg, 2.18 mmol) and acetic acid (131 mg, 2.18 mmol) were successively added and stirred at room temperature for 1 hour. Then sodium triacetoxyborohydride (578 mg, 2.73 mmol) was added and stirred overnight at room temperature. The reaction solution was concentrated, and pH was adjusted to 8 with saturated sodium bicarbonate solution, then extracted with dichloromethane. The organic phase was washed with saturated salt water, dried over anhydrous sodium sulfate, concentrated to obtain a crude product which was purified by column chromatography to obtain 138 mg of Compound C12-2.

(3) Synthesis of C12-3

The Compound C12-2 (138 mg, 0.44 mmol) was dissolved in methanol (5 ml), 10% wet Pd/C (13.8 mg) was added, and stirred for 2 hours under hydrogen atmosphere at room temperature. The reaction solution was filtered through diatomite, washed with methanol, and the filtrate was concentrated to obtain 97 mg Compound C12-3.

(4) Synthesis of Compound C14

The Compound C12-3 (69 mg, 0.26 mmol) and Compound C1-8 (85 mg, 0.27 mmol) were dissolved in ethylene glycol monomethyl ether (3 ml), then methanesulfonic acid (62 mg, 0.64 mmol) was added, heated to 90° C. and stirred overnight under nitrogen protection, then cooled, concentrated under reduced pressure to remove the solvent. The pH was adjusted to 8 with saturated sodium bicarbonate solution, extracted with dichloromethane, washed with saturated salt water, dried over anhydrous sodium sulfate, concentrated to obtain crude product. Certain amount of the crude product was purified by Prep-HPLC to obtain 22 mg C14. MS [M+H] 561.2.

¹H NMR (400 MHz, CDCl₃) δ 8.53-8.45 (m, 2H), 8.14 (s, 1H), 7.55-7.49 (m, 3H), 7.38-7.28 (m, 2H), 7.19-7.15 (m, 1H), 3.88 (s, 2H), 3.83 (s, 2H), 2.68 (s, 3H), 1.87-1.83 (d, 6H, J=13.12 Hz).

### Example 13 Synthesis of Compound C19

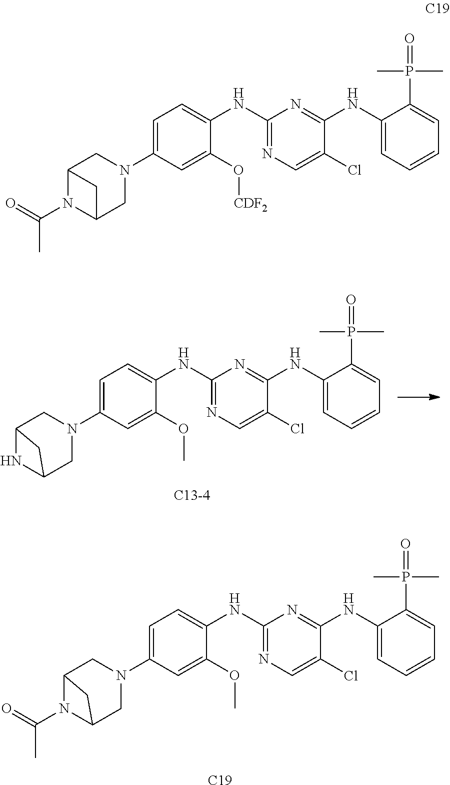

The Compound C13-4 (103 mg, 0.21 mmol) and acetyl chloride (21 mg, 0.27 mmol) were dissolved in DMF (5 ml), TEA (169 mg, 21 mmol) was added and stirred overnight at room temperature. The reaction solution was evaporated to dryness and purified by Prep-HPLC to obtain 30 mg Compound C19.

¹H NMR (400 MHz, CD₃OD) δ 8.35-8.32 (m, 1H), 7.99 (s, 1H), 7.58-7.51 (m, 1H), 7.41-7.37 (t, 1H), 7.22-7.18 (m, 1H), 6.40-6.39 (d, 1H, J=2.69 Hz), 6.25-6.23 (m, 1H), 4.69-4.68 (m, 1H), 4.59-4.56 (m, 1H), 3.84 (s, 3H), 3.81-3.79 (d, 1H, J=10.97 Hz), 3.71-3.61 (m, 1H), 3.52-3.42 (d, 1H, J=10.51 Hz), 2.80-2.75 (m, 1H), 1.95 (s, 3H), 1.85 (s, 3H), 1.81 (s, 3H), 1.75-1.73 (d, 1H, J=9.14 Hz).

### Example 14 Synthesis of Compound C20

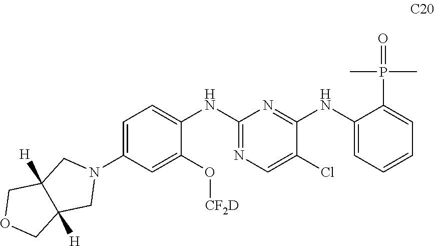

The experimental process was as follows:

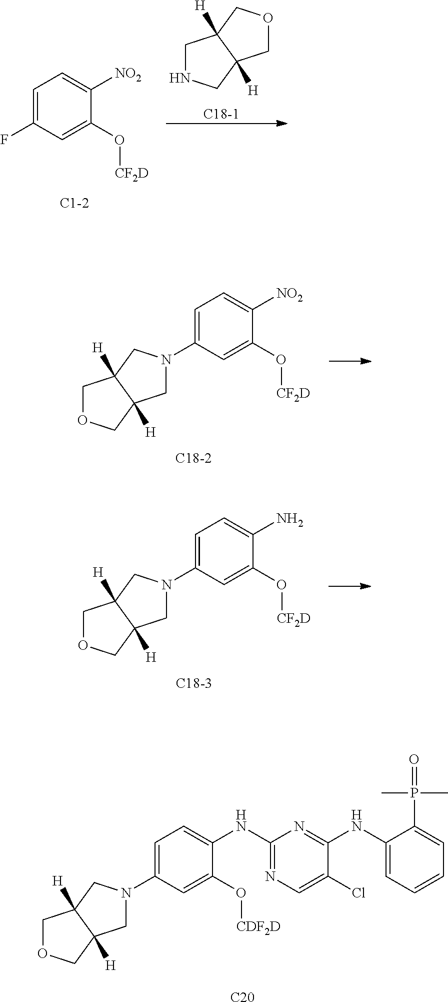

(1) Synthesis of C18-2

The Compound C1-2 (150 mg, 0.72 mmol) and Compound C18-1 (106 mg, 0.94 mmol) were dissolved in acetonitrile (5 ml), and potassium carbonate (219 mg, 1.59 mmol) was added, and then stirred overnight at 85° C. The solvent acetonitrile was removed by evaporation under reduced pressure, the residue was diluted with water, extracted with ethyl acetate, and the combined organic phases were washed with saturated salt water, dried over anhydrous sodium sulfate, concentrated to obtain crude product which was purified by column chromatography to obtain 325 mg of Compound C18-2.

¹H NMR (400 MHz, CDCl₃) δ 7.78-7.74 (q, 1H), 6.62-6.58 (dd, 1H, J=11.59 Hz, 2.59 Hz), 6.54-6.49 (m, 1H), 3.98-3.94 (m, 3H), 3.71-3.68 (dd, 2H, J=8.99 Hz, 3.81 Hz), 3.47-3.42 (m, 2H), 3.13-3.10 (dd, 2H, J=10.03 Hz, 3.03 Hz), 3.08-3.00 (m, 2H).

(2) Synthesis of C18-3

The Compound C18-2 (135 mg, 0.45 mmol) was dissolved in methanol (10 ml), then palladium hydroxide (20 mg) was added and stirred at room temperature under hydrogen atmosphere for 2h. The reaction solution was filtered through diatomite, washed with methanol, and the filtrate was concentrated to obtain 123 mg crude Compound C18-3 which was directly used for the next step without purification. MS [M+H] 317.2.

(3) Synthesis of Compound C20

The Compound C18-3 (146 mg, 0.54 mmol) and Compound C1-8 (175 mg, 0.56 mmol) were dissolved in ethylene glycol monomethyl ether (4 ml), then 4N HCl/dioxane (0.4 ml, 1.62 mmol) was added, stirred overnight at 120° C., concentrated under reduced pressure. pH was adjusted to 8 with saturated sodium bicarbonate solution, extracted with dichloromethane. The combined organic phases were washed with saturated salt water, dried over anhydrous sodium sulfate, concentrated to obtain crude product. Certain amount of the crude product was purified by Prep-HPLC to obtain 50 mg Compound C20.

¹H NMR (400 MHz, CD₃OD) δ 8.29-8.25 (q, 1H), 8.00 (s, 1H), 7.57-7.51 (m, 1H), 7.43-7.41 (d, 1H, J=9.02 Hz), 7.36-7.32 (t, 1H), 7.19-7.15 (m, 1H), 6.53-6.46 (m, 2H), 3.99-3.96 (m, 2H), 3.70-3.67 (m, 2H), 3.45-3.41 (m, 2H), 3.25-3.22 (m, 2H), 3.11-3.08 (m, 2H), 1.85-1.81 (d, 6H, J=13.37 Hz).

### Example 15 Synthesis of Compound C22

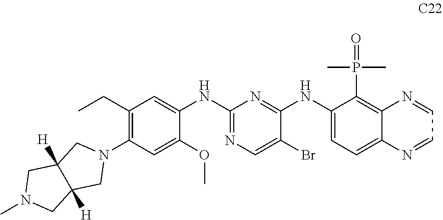

The experimental process was as follows:

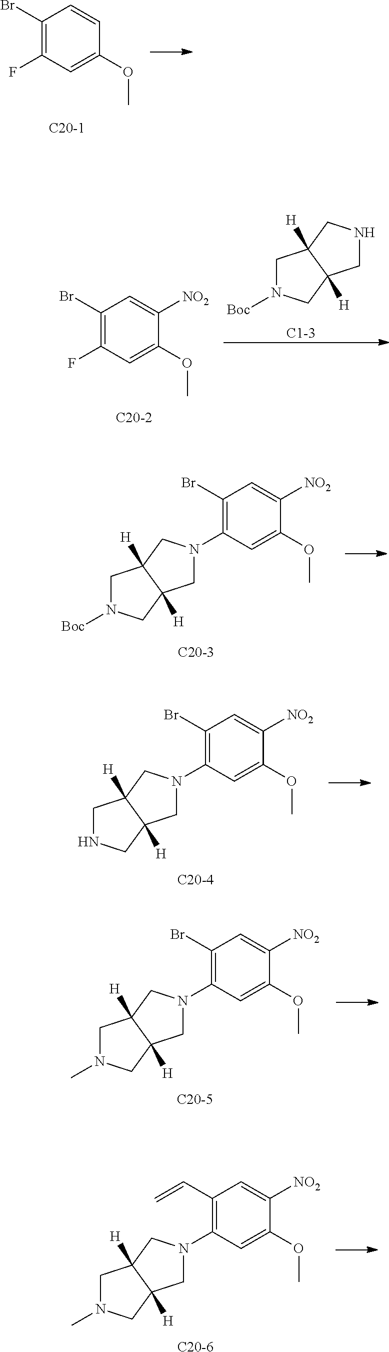

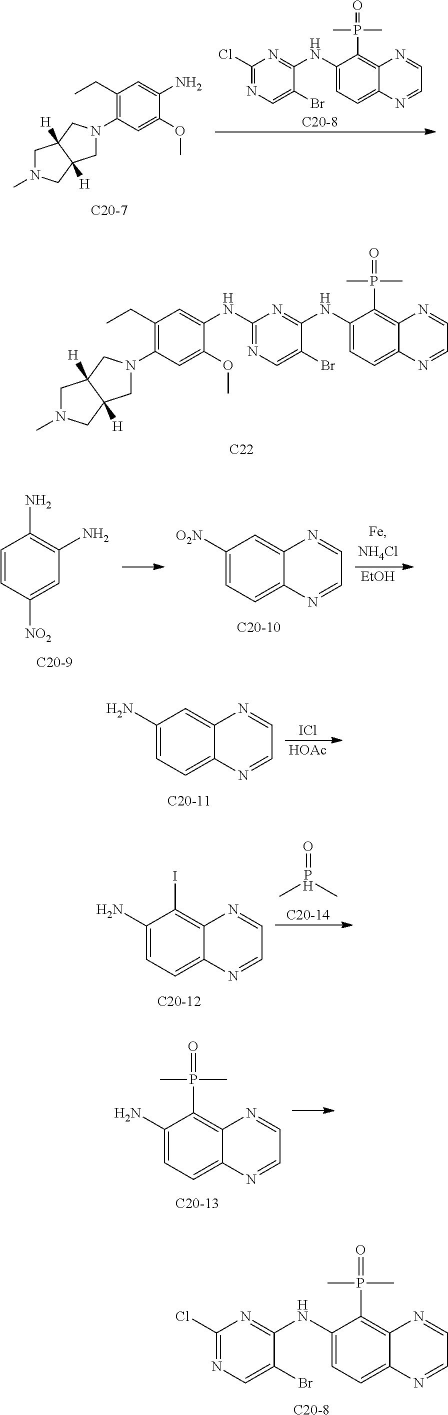

(1) Synthesis of C20-2

The Compound C20-1 (4.57 g, 22 mmol) was dissolved in concentrated H₂SO₄ (20 ml), then cooled to 0° C. Potassium nitrate (2.2 g, 22 mmol) was added in batches, and then stirred at 0° C. for 30 min. The reaction solution was quenched in crushed ice, extracted with ethyl acetate. The combined organic phases were washed with saturated salt water, dried over anhydrous sodium sulfate, concentrated to obtain a crude product which was purified by column chromatography to obtain 1.68 g of Compound C20-2.

¹H NMR (400 MHz, CD₃OD) δ 8.20-8.18 (d, 1H, J=7.30 Hz), 7.30-7.27 (d, 1H, J=10.48 Hz), 3.97 (s, 3H).

(2) Synthesis of C20-3

The Compound C20-2 (300 mg, 1.25 mmol) and Compound C1-3 (319 mg, 1.51 mmol) were dissolved in DMF (10 ml), and potassium carbonate (513 mg, 3.76 mmol) was added, and then stirred overnight at 80° C. The reaction solution was evaporated to dryness, and water was added, extracted with ethyl acetate. The organic phases were washed with saturated salt water, dried over anhydrous sodium sulfate, concentrated to obtain crude product which was purified by column chromatography to obtain 600 mg Compound C20-3.

¹H NMR (400 MHz, CDCl₃) δ 8.27 (s, 1H), 6.22 (s, 1H), 3.95 (s, 3H), 3.84 (br, 2H), 3.65 (br, 2H), 3.55-3.52 (m, 2H), 3.41-3.30 (m, 2H), 2.99 (br, 2H), 1.47 (s, 9H).

(3) Synthesis of C20-4

The Compound C20-3 (600 mg, 1.36 mmol) was dissolved in TFA (20 ml) and stirred at room temperature for 4h. The reaction solution was evaporated to dryness to obtain 545 mg of Compound C20-4.

¹H NMR (400 MHz, CD₃OD) δ 8.15 (s, 1H), 6.74 (s, 1H), 3.98 (s, 3H), 3.75-3.72 (d, 2H, J=9.94 Hz), 3.69-3.65 (m, 2H), 3.27-3.17 (m, 6H).

(4) Synthesis of C20-5

The Compound C20-4 (200 mg, 0.58 mmol) and 37% formaldehyde solution (189 mg, 2.34 mmol) were dissolved in THF (40 ml), acetic acid (140 mg, 2.34 mmol) was added and stirred at room temperature for 1 h, then sodium borohydride acetate (620 mg, 2.92 mmol) was added and stirred overnight at room temperature. The reaction solution was evaporated to dryness, saturated sodium bicarbonate solution was added to adjust pH to >7, extracted with ethyl acetate. The combined organic phases were washed with saturated salt water, dried over anhydrous sodium sulfate, concentrated to obtain crude product which was purified by column chromatography to obtain 170 mg Compound C20-5.

¹H NMR (400 MHz, CDCl₃) δ 8.23 (s, 1H), 6.44 (s, 1H), 3.94 (s, 3H), 3.60-3.55 (m, 2H), 3.32-3.29 (m, 2H), 2.96-2.92 (m, 2H), 2.72-2.68 (m, 2H), 2.52-2.49 (m, 2H), 2.36 (s, 3H).

(5) Synthesis of C20-6

The Compound C20-5 (455 mg, 1.28 mmol) and tributylvinylstannane (809 mg, 2.56 mmol) were dissolved in toluene (20 ml), then triphenylphosphine palladium chloride (89.7 mg, 0.13 mmol), cuprous bromide (55.1 mg, 0.39 mmol), and triphenylphosphine (101 mg, 0.39 mmol) were added, and stirred overnight under argon at 110° C. Then the reaction solution was quenched with potassium fluoride solution, extracted with ethyl acetate. The combined organic phases were washed with saturated salt water, dried over anhydrous sodium sulfate, concentrated to obtain crude product which was purified by column chromatography to obtain 530 mg Compound C20-6.

¹H NMR (400 MHz, CDCl3) δ 8.04 (s, 1H), 6.77-6.70 (m, 1H), 6.37 (s, 1H), 5.63-5.59 (dd, 1H, J=17.59 Hz, 1.11 Hz), 5.29-5.26 (dd, 1H, J=10.80 Hz, 0.97 Hz), 3.95 (s, 3H), 3.41-3.36 (m, 2H), 3.17-3.14 (m, 2H), 2.95-2.94 (m, 2H), 2.77-2.74 (m, 2H), 2.50-2.48 (m, 2H), 2.39 (s, 3H).

(6) Synthesis of C20-7

The Compound C20-6 (100 mg, 0.33 mmol) was dissolved in methanol (10 ml), palladium hydroxide (40 mg) was added, two drops of acetic acid were added, and stirred at 50° C. for 4 days. The reaction solution was filtered through diatomite and concentrated to obtain 80 mg of Compound C20-7. [M+H]: 276.2.

¹H NMR (400 MHz, CDCl₃) δ 6.65 (s, 1H), 6.58 (s, 1H), 3.82 (s, 3H), 3.13 (br, 2H), 2.92-2.85 (m, 4H), 2.77 (s, 3H), 2.66-2.53 (m, 4H), 1.63-1.57 (m, 2H), 1.20-1.17 (t, 3H).

(7) Synthesis of C20-10

The Compound C20-9 (10 g, 65.3 mmol) was dissolved in ethanol (100 ml), then 40% acetaldehyde aqueous solution (9 ml, 78.4 mmol) was added, and the temperature was raised to 80° C. and stirred overnight. After the reaction was completed, the reaction solution was cooled, the solids in the reaction solution were filtered out, and the filter cake was washed with a small amount of ethanol. 9.25 g of Compound C20-10 was obtained after vacuum drying.

¹H NMR (400 MHz, DMSO-d6) δ 9.17 (s, 2H), 8.93-8.92 (d, 1H, J=2.82 Hz), 8.60-8.57 (dd, 1H, J=9.17 Hz, 2.47 Hz), 8.37-8.35 (d, 1H, J=9.17 Hz).

(8) Synthesis of C20-11

The Compound C20-10 (9.25 g, 53 mmol) was dissolved in ethanol (180 ml), then reduced iron powder (17.76 g, 317 mmol) and 62 ml aqueous ammonium chloride solution (28.3 g solid ammonium chloride was dissolved in 62 ml water) were added, and stirred at 90° C. for 3 hours under the protection of nitrogen. After the reaction was completed, the reaction solution was cooled and filtered through diatomite, and the filtrate was concentrated to obtain 24.5 g of crude Compound C20-11 (Containing a large amount of ammonium chloride). Ms [M+H] 146.2.

(9) Synthesis of C20-12

The crude Compound C20-11 (24.5 g, 53 mmol) was dissolved in glacial acetic acid (200 ml), iodine chloride (9.4 g, 58 mmol) was added and stirred at room temperature for 1 hour. After the reaction was completed, the solvent was removed by evaporation under reduced pressure and diluted with ethyl acetate. The organic phase was washed with saturated salt water, dried over anhydrous sodium sulfate, concentrated, and purified by column chromatography to obtain 7.1 g of Compound C20-12.

¹H NMR (400 MHz, DMSO-d6) δ 8.72-8.71 (d, 1H, J=1.90 Hz), 8.52-8.51 (d, 1H, J=1.90 Hz), 7.78-7.76 (d, 1H, J=9.00z), 7.43-7.41 (d, 1H, J=9.00 Hz), 6.32 (br, 2H).

(10) Synthesis of C20-13

The Compound C20-12 (6.4 g, 23.58 mmol) and Compound C20-14 (2.76 g, 35.37 mmol) was dissolved in a mixed solution of DMF (300 ml) and water (60 ml), then palladium acetate (0.53 g, 2.36 mmol), Xant-phos (1.37 g, 2.36 mmol) and potassium phosphate (7.5 g, 35.37 mmol) were successively added. The reaction solution was stirred at 120° C. for 24 hours under the protection of nitrogen gas. After the reaction was completed, the reaction solution was cooled to room temperature, concentrated and diluted with ethyl acetate. The organic phase was washed with saturated salt water, dried over anhydrous sodium sulfate, concentrated, and purified by column chromatography to obtain 2.3 g of Compound C20-13.

¹H NMR (400 MHz, CDCl₃) δ 8.56-8.55 (d, 1H, J=1.98 Hz), 8.49-8.48 (d, 1h, J=1.98 Hz), 7.88-7.85 (d, 1H, J=9.35 Hz), 7.04-*7.01 (q, 1H), 2.03-1.99 (d, 6H, J=13.82 Hz).

(11) Synthesis of C20-8

The Compound C20-13 (500 mg, 2.26 mmol) was dissolved in 10 ml DMF, cooled to 0° C., 60% NaH (181 mg, 4.52 mmol) was added in batches, and stirred at 0° C. for 1 h. 5-bromo-2, 4-dichloropyrimidine (1.02 g, 4.52 mmol) was added, and the temperature was raised to room temperature, and stirred overnight. After the reaction was completed, water was added to quench and extracted with dichloromethane. The organic phase was washed with saturated salt water, dried over anhydrous sodium sulfate, concentrated, and purified by column chromatography to obtain 231 mg of Compound C20-8.

¹H NMR (400 MHz, CDCl₃) δ 13.07 (br, 1H), 9.05-9.01 (m, 1H), 8.82-8.81 (d, 1H, J=1.86 Hz), 8.75-8.74 (d, 1h, J=1.93 Hz), 8.42 (s, 1H), 8.28-8.25 (d, 1H, J=9.64 Hz), 2.15-2.12 (d, 6H, J=14.39 Hz).

(12) Synthesis of Compound C22

The Compound C20-7 (113 mg, 0.41 mmol) and Compound C20-8 (177 mg, 0.41 mmol) were dissolved in ethylene glycol monomethyl ether (10 ml), methanesulfonic acid (118 mg, 1.23 mmol) was added, and stirred overnight under argon at 90° C. The reaction solution was purified by Prep-HPLC to obtain 16 mg Compound C22.

¹H NMR (400 MHz, CD₃OD) δ 8.88-8.84 (m, 2H), 8.80-8.79 (d, 1H, J=2.03 Hz), 8.23 (s, 1H), 7.97-7.94 (d, 1H, J=9.56 Hz), 7.64 (s, 1H), 6.83 (s, 1H), 3.85 (s, 3H), 3.63-3.56 (m, 4H), 3.12-3.00 (m, 8H), 2.84 (s, 3H), 2.57-2.50 (m, 2H), 2.15 (s, 3H), 2.12 (s, 3H), 0.90-0.86 (t, 3H).

### Example 16 Synthesis of Compound C23

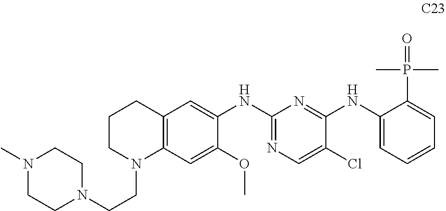

The experimental process was as follows:

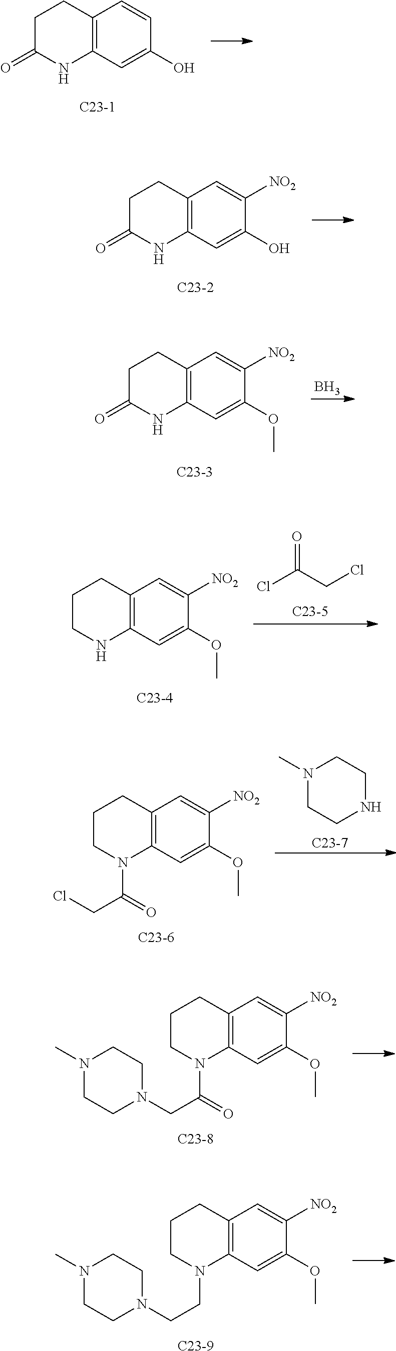

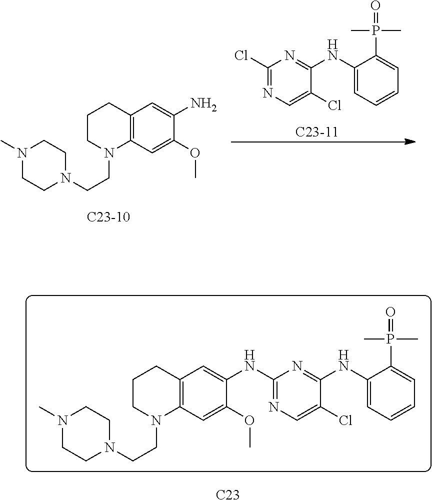

(1) Synthesis of Compound C23-2

Compound C23-1 (1.64 g, 10 mmol) was dissolved in 30 ml concentrated H₂SO₄, cooled to 0° C., and H₂O (7.6 ml) was added dropwise slowly, then HNO₃ (65%, 0.76 ml) was added dropwise slowly at 0° C. and stirred for 15 min. The reaction solution was quenched in crushed ice, extracted with ethyl acetate. The combined organic phases were washed with saturated salt water, dried over anhydrous sodium sulfate, concentrated to obtain a crude product which was purified by column chromatography to obtain 837 mg of Compound C23-2.

1H NMR (400 MHz, DMSO-d6) δ 10.36 (br, 1H), 7.76 (s, 1H), 6.37 (s, 1H), 2.82-2.78 (t, 2H), 2.47-2.43 (t, 2H).

(2) Synthesis of Compound C23-3

The Compound C23-2 (837 mg, 4.02 mmol) was dissolved in 6 ml DMF and K₂CO₃ (833 mg, 4.83 mmol) and CH₃I (0.32 ml, 6.03 mmol) were added, then stirred overnight at room temperature. Water was added to the reaction solution and pH was adjusted to <7 with HCl, extracted with ethyl acetate. The organic phases were washed with saturated salt water, dried over anhydrous sodium sulfate, concentrated to obtain crude product which was purified by column chromatography to obtain 419 mg Compound C23-3.

1H NMR (400 MHz, DMSO-d6) δ 10.49 (br, 1H), 7.88 (s, 1H), 6.72 (s, 1H), 3.86 (s, 3H), 2.91-2.88 (t, 2H), 2.53-2.49 (t, 2H).

(3) Synthesis of Compound C23-4

The Compound C23-3 (600 mg, 0.9 mmol) was dissolved in 6 ml THF and 1N BH₃/THF (20 ml, 20 mmol) was added, then stirred at 80° C. for 30 min. Cooled to 0° C., then 1N HCl (6 ml) was added, and stirred for 30 min. pH was adjusted to neutral with NaHCO₃, extracted with ethyl acetate. The combined organic phases were washed with saturated salt water, dried over anhydrous sodium sulfate, concentrated to obtain crude product which was purified by column chromatography to obtain 327 mg of Compound C23-4.

1H NMR (400 MHz, CDCl₃) δ 7.80 (s, 1H), 5.94 (s, 1H), 4.69 (br, 1H), 3.87 (s, 3H), 3.40-3.36 (t, 2H), 2.72-2.69 (t, 2H), 1.94-1.91 (m, 2H).

(4) Synthesis of Compound C23-6

The Compound C23-4 (50 mg, 0.24 mmol) was dissolved in 1 ml ethyl acetate, and Compound C23-5 (54 mg, 0.48 mmol) was added dropwise. Then the temperature was raised to 60° C. and stirred for 1 h. TLC detection showed the reaction was completed, and the reaction solution was evaporated to dryness to obtain 71 mg of Compound C23-6.

1H NMR (400 MHz, MeOD-d3) δ 7.78 (s, 1H), 7.67 (s, 1H), 4.53 (s, 2H), 3.96 (s, 3H), 3.89-3.86 (t, 2H), 2.85-2.82 (t, 2H), 2.09-2.03 (m, 2H).

(5) Synthesis of Compound C23-8

The Compound C23-6 (53 mg, 0.19 mmol) was dissolved in 1 ml THF and Compound C23-7 (37 mg, 0.37 mmol) was stirred overnight at room temperature, and purified on preparation plate to obtain 50 mg of Compound C23-8.

1H NMR (400 MHz, CDCl₃) δ 7.80 (s, 1H), 7.74 (s, 1H), 3.93 (s, 3H), 3.82-3.80 (t, 2H), 3.40 (s, 2H), 2.80-2.77 (t, 2H), 2.74-2.68 (m, 8H), 2.05-1.98 (m, 2H).

(6) Synthesis of Compound C23-9

The Compound C23-8 (158 mg, 0.45 mmol) was dissolved in 5 ml THF, then 2.7 ml 1N BH₃/THF was added and stirred overnight at room temperature. The reaction solution was quenched by adding 1N HCl dropwise. The pH was adjusted to >7 with saturated sodium bicarbonate solution. The reaction solution was extracted with ethyl acetate, washed with saturated salt water, dried over anhydrous sodium sulfate, and purified by column chromatography to obtain 64 mg of Compound C23-9.

1H NMR (400 MHz, CDCl₃) δ 7.78 (s, 1H), 6.08 (s, 1H), 3.92 (s, 3H), 3.51-3.47 (t, 2H), 3.51-3.47 (t, 2H), 2.71-2.67 (t, 2H), 2.62-2.58 (t, 2H), 2.54-2.47 (m, 8H), 2.30 (s, 3H), 1.96-1.90 (m, 2H).

(7) Synthesis of Compound C23-10

The Compound C23-9 (64 mg) was dissolved in 4 mL methanol and 2 mL ethyl acetate, Pd/C (7 mg) was added, and stirred overnight at room temperature under hydrogen atmosphere. The reaction solution was filtered through diatomite and concentrated to obtain 50 mg of Compound C23-10. [M+H]: 305.3.

(8) Synthesis of Compound C23

Compound C23-10 (50 mg, 0.16 mmol) and Compound C23-11 (67 mg, 0.21 mmol) was dissolved in 2 ml ethylene glycol monomethyl ether, and 4N HCl/Dioxnae (0.13 ml, 0.5 mmol) was added, then stirred overnight under the protection of argon at 120° C., and purified by Prep-HPLC to obtain 17 mg of Compound C23.

1H NMR (400 MHz, MeOD-d3) δ 8.34-8.31 (m, 1H), 8.01 (s, 1H), 7.61-7.56 (m, 1H), 7.45-7.41 (t, 1H), 7.24-7.19 (m, 2H), 6.31 (s, 1H), 3.80 (s, 3H), 3.64-3.40 (m, 4H), 2.88-2.69 (m, 10H), 2.56 (s, 3H), 2.01-1.86 (m, 4H), 1.84 (s, 3H), 1.80 (s, 3H).

### Example 17 Synthesis of Compound C24

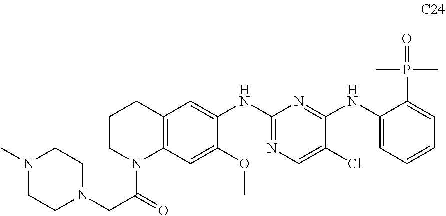

The experimental process was as follows:

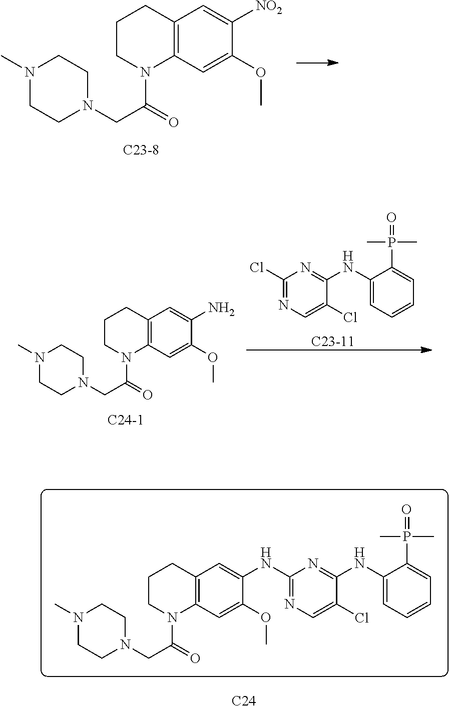

(1) Synthesis of Compound C24-1

The Compound C23-8 (100 mg) was dissolved in 5 mL methanol, Pd/C (10 mg) was added, and stirred for 2h at room temperature under hydrogen atmosphere. The reaction solution was filtered through diatomite and concentrated to obtain 90 mg of Compound C24-1. [M+H]: 319.3.

(2) Synthesis of Compound C24

The Compound C24-1 (90 mg, 0.28 mmol) and Compound C23-11 (115 mg, 0.37 mmol) was dissolved in 4 ml ethylene glycol monomethyl ether, and 4N HCl/Dioxnae (1 ml, 4 mmol) was added, then stirred overnight under the protection of argon at 120° C., and purified by Prep-HPLC to obtain 26 mg of Compound C24.

1H NMR (400 MHz, MeOD-d3) δ 8.24-8.20 (m, 1H), 8.11 (s, 1H), 7.79 (s, 1H), 7.71-7.65 (m, 1H), 7.60-7.56 (t, 1H), 7.35-7.31 (m, 2H), 3.87 (s, 3H), 3.78-3.75 (t, 2H), 3.54 (s, 2H), 2.88-2.75 (m, 8H), 2.53-2.49 (m, 5H), 1.96-1.92 (m, 2H), 1.86 (s, 3H), 1.82 (s, 3H).

### Example 18 Synthesis of Compound C25

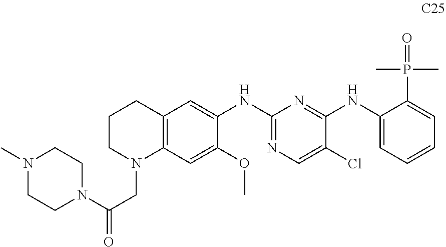

The experimental process was as follows:

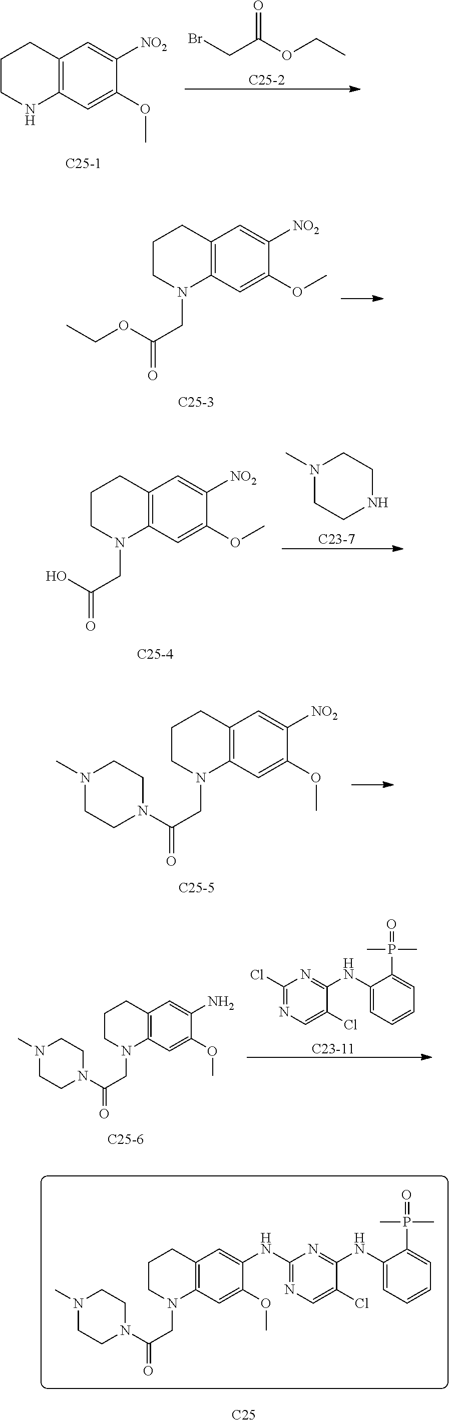

(1) Synthesis of Compound C25-3

Compound C25-1 (327 mg, 1.57 mmol) was dissolved in 30 ml DMF and Cs₂CO₃ (1.5 g, 4.71 mmol) and Compound C25-2 (0.4 ml, 3.14 mmol) were added, the temperature was raised to 80° C., stirred overnight, concentrated. Water was added, and extracted with ethyl acetate. The combined organic phases were washed with saturated salt water, dried over anhydrous sodium sulfate, and concentrated to obtain crude product which was purified by column chromatography to obtain 225 mg of Compound C25-3.

1H NMR (400 MHz, CDCl₃) δ 7.69 (s, 1H), 5.82 (s, 1H), 4.17-4.11 (m, 2H), 4.00 (s, 2H), 3.79 (s, 3H), 3.42-3.39 (t, 2H), 2.67-2.64 (m, 2H), 1.94-1.88 (m, 2H), 1.21-1.17 (t, 3H).

(2) Synthesis of Compound C25-4

The Compound C25-3 (315 mg, 1.07 mmol) was dissolved in 20 ml THF, and 1N NaOH (2.4 ml, 2.4 mmol) was added, and stirred overnight at room temperature. Then 1N HCl was added to adjust pH to <7, extracted with ethyl acetate. The combined organic phases were washed with saturated salt water, dried over anhydrous sodium sulfate, and concentrated to obtain 245 mg of Compound C25-4.

1H NMR (400 MHz, CDCl₃) δ 7.79 (s, 1H), 5.92 (s, 1H), 4.12 (s, 2H), 3.89 (s, 3H), 3.49-3.46 (t, 2H), 2.75-2.72 (t, 2H), 2.01-1.97 (m, 2H).

(3) Synthesis of Compound C25-5

The Compound C25-4 (245 mg, 0.92 mmol), Compound C23-7 (96 mg, 0.96 mmol) and TEA (186 mg, 1.84 mmol) were dissolved in 2 ml acetonitrile, and HATU (364 mg, 0.96 mmol) was added, stirred at room temperature for 30 min, washed with 1N HCl, washed with water, and then washed with saturated sodium bicarbonate solution, dried over anhydrous sodium sulfate, and filtered through diatomite and concentrated to obtain 280 mg of Compound C25-5.

1H NMR (400 MHz, MeOD-d3) δ 7.70 (s, 1H), 5.89 (s, 1H), 4.37 (S, 2H), 3.84 (s, 3H), 3.63-3.59 (m, 4H), 3.41-3.38 (t, 2H), 2.73-2.70 (t, 2H), 2.53-2.50 (t, 2H), 2.45-2.43 (t, 2H), 2.32 (s, 3H), 1.99-1.93 (m, 2H).

(4) Synthesis of Compound C25-6

Compound C25-5 (100 mg) was dissolved in 5 ml methanol and 5 ml ethyl acetate, Pd/C (10 mg) was added, and stirred overnight at room temperature under hydrogen atmosphere. The reaction solution was filtered through diatomite and concentrated to obtain 88 mg of Compound C25-6. [M+H]: 319.3.

(5) Synthesis of Compound C25

The Compound C25-6 (88 mg, 0.28 mmol) and Compound C23-11 (115 mg, 0.37 mmol) was dissolved in 4 mL ethylene glycol monomethyl ether, and 4N HCl/Dioxnae (1 mL, 1 mmol) was added, then stirred at 120° C. overnight under the protection of argon, and purified by Prep-HPLC to obtain 20 mg of Compound C25.

1H NMR (400 MHz, MeOD-d3) δ 8.35-8.32 (m, 1H), 8.11 (s, 1H), 7.98 (s, 1H), 7.61-7.56 (m, 1H), 7.50-7.46 (t, 1H), 7.25-7.20 (m, 2H), 6.12 (s, 1H), 4.27-4.25 (m, 2H), 4.11 (s, 2H), 3.75 (s, 3H), 3.58-3.55 (m, 2H), 3.38-3.31 (m, 9H), 2.58-2.55 (m, 2H), 1.95-1.92 (m, 2H), 1.86 (s, 3H), 1.82 (s, 3H).

### Example 19 Synthesis of Compound C26

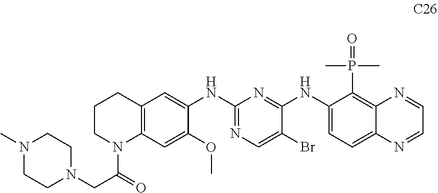 [SMILES: COc1cc2c(cc1Nc1ncc(Br)c(Nc3ccc4nccnc4c3P(C)(C)=O)n1)CCCN2C(=O)CN1CCN(C)CC1]

The experimental process was as follows:

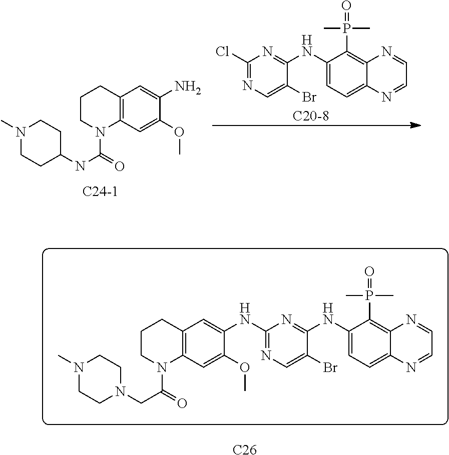

(1) Synthesis of Compound C26

The Compound C24-1 (45 mg, 0.14 mmol) and Compound C20-8 (76 mg, 0.18 mmol) was dissolved in 4 mL ethylene glycol monomethyl ether, and 4N HCl/Dioxnae (0.1 mL, 0.42 mmol) was added, then stirred at 90° C. overnight under the protection of argon, and purified by Prep-HPLC to obtain 11 mg of Compound C26.

1H NMR (400 MHz, CD₃Cl₃) 12.57 (s, 1H), 9.01-8.98 (dd, 1H, J=9.45 Hz J=3.78 Hz), 8.77-8.76 (d, 1H, J=1.89 Hz), 8.73-8.72 (d, 1H, J=1.89 Hz), 8.29 (s, 1H), 8.12-8.10 (d, 1H, J=9.56 Hz), 8.05 (br, 1H), 7.54 (s, 1H), 3.86 (s, 3H), 3.76-3.73 (t, 2H), 3.20 (s, 2H), 2.65-2.54 (m, 10H), 2.32 (s, 3H), 2.14 (s, 3H), 2.11 (s, 3H), 1.93-1.90 (t, 2H).

### Example 20 Synthesis of Compound C27

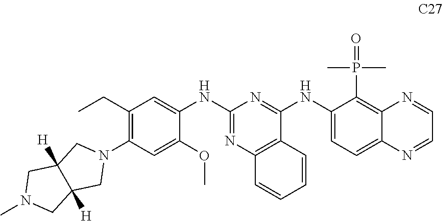 [SMILES: CCc1cc(Nc2nc(Nc3ccc4nccnc4c3P(C)(C)=O)c3ccccc3n2)c(OC)cc1N1C[C@H]2CN(C)C[C@H]2C1]

The experimental process was as follows:

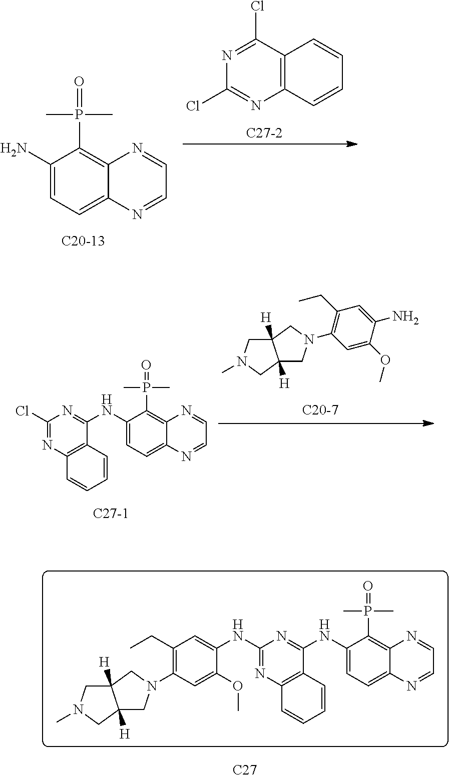

(1) Synthesis of Compound C27-1

The Compound C20-13 (100 mg, 0.452 mmol) was dissolved in 3 ml anhydrous DMF, then 60% sodium hydride (91 mg, 2.26 mmol) was added, and stirred at room temperature for 1 hour. Then Compound C27-2 (269 mg, 1.36 mmol) was added and stirred overnight at room temperature, then quenched with saturated salt water. The reaction solution was extracted with ethyl acetate, washed with saturated salt water, dried over anhydrous sodium sulfate, concentrated and purified by column chromatography to obtain 120 mg of Compound C27-1.

1H NMR (400 MHz, CDCl₃) δ 13.93 (s, 1H), 9.72-9.68 (q, 1H), 8.82-8.76 (dd, 2H, J=23.83 Hz, 1.83 Hz), 8.52-8.50 (d, 1H, J=7.94 Hz), 8.35-8.33 (d, 1H, J=9.78 Hz), 7.87-7.83 (m, 2H), 7.67-7.63 (m, 1H), 2.21 (s, 3H), 2.17 (s, 3H).

(2) Synthesis of Compound C27

The Compound C27-1 (86 mg, 0.26 mmol) and Compound C20-7 (68 mg, 0.247 mmol) were dissolved in 2 ml of ethylene glycol monomethyl ether, and then 4M hydrochloride in dioxane solution (0.15 ml, 0.618 mmol) was added. The temperature was raised to 120° C. and stirred overnight under the protection of nitrogen. After the reaction was completed, the reaction solution was concentrated, saturated sodium bicarbonate solution was added and pH was adjusted to be alkaline, and extracted with ethyl acetate. The organic phase was washed with saturated salt water, dried over anhydrous sodium sulfate, concentrated, and purified by Prep-HPLC to obtain 30 mg of the target compound.

1H NMR (400 MHz, CDCl₃) δ 13.45 (s, 1H), 9.66-9.63 (q, 1H), 8.51 (s, 1H), 8.34-8.32 (d, 1H, J=8.28 Hz), 8.21-8.18 (d, 1H, J=10.12 Hz), 7.68-7.62 (m, 2H), 7.43 (br, 1H), 7.35-7.33 (t, 1H), 6.73 (s, 1H), 3.90 (s, 3H), 3.35 (br, 1H), 3.07-2.99 (m, 5H), 2.9-74-2.68 (q, 2H), 2.61-2.53 (m, 4H), 2.19 (s, 3H), 2.15 (s, 3H), 1.27-1.24 (t, 3H).

### Example 21 Synthesis of Compound C28

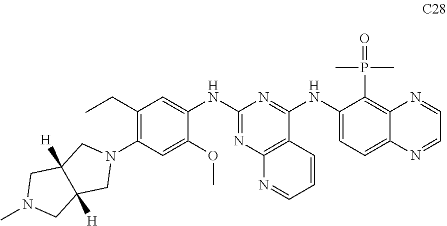 [SMILES: CCc1cc(Nc2nc(Nc3ccc4nccnc4c3P(C)(C)=O)c3cccnc3n2)c(OC)cc1N1C[C@H]2CN(C)C[C@H]2C1]

The experimental process was as follows:

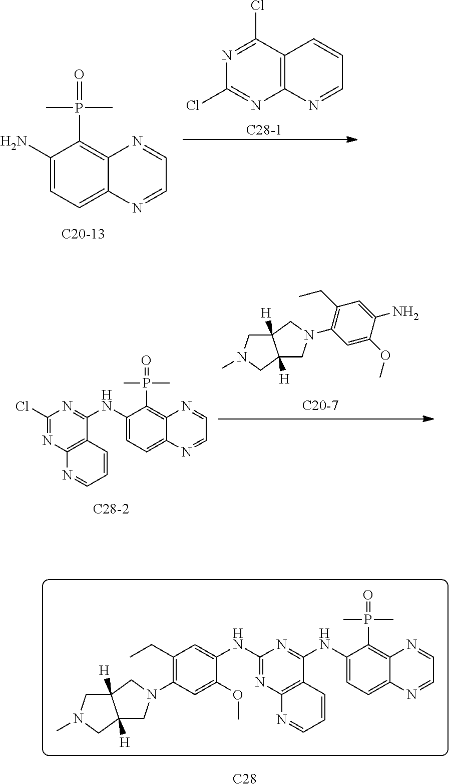

(1) Synthesis of Compound C28-2

The Compound C20-13 (80 mg, 0.362 mmol) was dissolved in 5 mL anhydrous DMF, then 60% sodium hydride (64 mg, 1.6 mmol) was added, and stirred at room temperature for 1 hour. Then Compound C28-1 (217 mg, 1.09 mmol) was added and stirred overnight at room temperature, then quenched with saturated salt water. The reaction solution was extracted with ethyl acetate, washed with saturated salt water, dried over anhydrous sodium sulfate, concentrated and purified by column chromatography to obtain 77 mg of Compound C28-2.

1H NMR (400 MHz, CDCl₃) δ 9.76-9.72 (q, 1H), 9.12-9.10 (q, 1H), 8.93-8.91 (dd, 1H, J=8.22 Hz, 1.57 Hz), 8.86-8.79 (dd, 1H, J=23.63 Hz, 1.67 Hz), 8.40-8.37 (d, 1H, J=9.45 Hz), 7.61-7.57 (q, 1H), 2.22 (s, 3H), 2.18 (s, 3H).

(2) Synthesis of Compound C28

The Compound C28-2 (91 mg, 0.24 mmol) and Compound C20-7 (62 mg, 0.23 mmol) were dissolved in 3 mL ethylene glycol monomethyl ether, then 4M hydrochloride in dioxane solution (0.14 ml, 0.565 mmol) was added. The temperature was heated to 120° C. and stirred overnight under the protection of nitrogen. After the reaction was completed, the reaction solution was concentrated. pH was adjusted to be alkaline with saturated sodium bicarbonate solution, and extracted with ethyl acetate. The organic phase was washed with saturated salt water, dried over anhydrous sodium sulfate, concentrated, and purified by Prep-HPLC to obtain 24 mg of Compound C28.

1H NMR (400 MHz, CDCl₃) δ 13.67 (s, 1H), 8.92 (s, 1H), 8.80-8.69 (m, 3H), 8.20 (br, 1H), 7.61 (s, 1H), 7.24-7.22 (m, 1H), 6.74 (s, 1H), 3.89 (s, 3H), 3.33 (d, 2H, J=7.09 Hz), 3.02 (br, 6H), 2.73-2.53 (m, 8H), 2.20 (s, 3H), 2.16 (s, 3H), 1.28-1.25 (t, 3H).

### Example 22 Synthesis of Compound C29

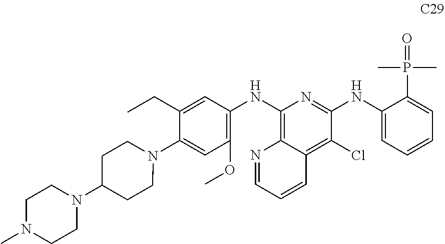

The experimental process was as follows:

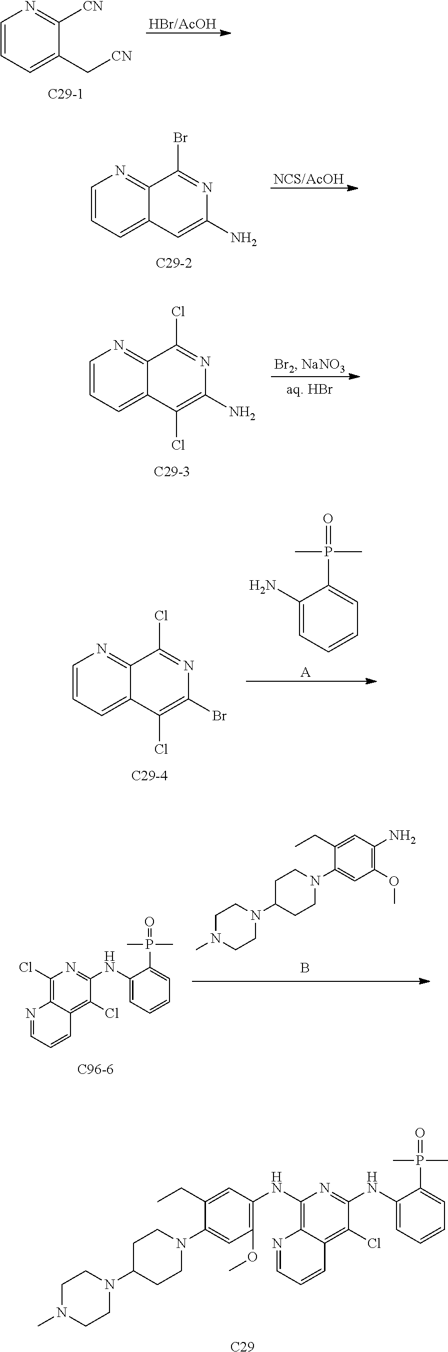

(1) Synthesis of Compound C29-2

The Compound C29-1 (5.0 g, 35 mmol) was added to 30 mL solution of hydrobromic acid and acetic acid in batches at a temperature not higher than 30° C., and then the reaction solution was stirred at room temperature for 2 hours. After the reaction was completed, the reaction solution was adjusted to be alkaline with saturated sodium bicarbonate solution, and extracted with ethyl acetate, washed with saturated salt water, dried over anhydrous sodium sulfate, and concentrated to obtain 6.2 g crude Compound C29-2 which was directly used for the next step without purification.

1H NMR (400 MHz, CDCl₃) δ 8.60-8.59 (q, 1H), 8.03-8.01 (q, 1H), 7.50-7.47 (q, 1H), 6.60 (s, 1H), 6.47 (s, 2H).

(2) Synthesis of Compound C29-3

The Compound C29-2 (3.0 g, 13.4 mmol) was dissolved in HOAc (20 ml), then NCS (2.68 g, 20.1 mmol) was added. The temperature was heated to 50° C., stirred overnight. After the reaction was completed, the reaction solution was cooled to room temperature, concentrated and evaporated under reduced pressure. Then saturated sodium bicarbonate was added to adjust the pH to be alkaline, extracted with ethyl acetate, washed with saturated salt water, dried over anhydrous sodium sulfate, concentrated, and purified by column chromatography to obtain 900 mg of Compound C29-3.

1H NMR (400 MHz, DMSO-d₃) δ 8.73-8.72 (t, 1H), 8.24-8.22 (t, 1H), 7.74-7.71 (q, 1H), 6.90 (br, 2H).

(3) Synthesis of Compound C29-4

The Compound C29-3 (463 mg, 2.16 mmol) was dissolved in 5 ml aqueous hydrobromic acid solution, and cooled to −5° C. under an ice salt bath. Liquid bromine (1.04 g, 6.49 mmol) was added and stirred for 30 min, then sodium nitrite (373 mg, 5.4 mmol) was added and stirred for 30 min. The temperature was raised to room temperature and stirred for 1 hour. After the reaction was completed, 10% sodium hydroxide solution was added to adjust pH to be neutral, extracted with ethyl acetate, dried over anhydrous sodium sulfate, concentrated, and purified by column chromatography to obtain 395 mg of Compound C29-4.

1H NMR (400 MHz, CDCl₃) δ 9.19-9.18 (q, 1H), 8.61-8.58 (q, 1H), 7.84-7.80 (q, 1H).

(4) Synthesis of Compound C29-6

The Compound C29-4 (200 mg, 0.72 mmol) and Compound A (101 mg, 0.6 mmol) were dissolved in 10 ml of anhydrous dioxane, and Pd₂(dba)₃ (55 mg, 0.06 mmol), Xantphos (70 mg, 0.12 mmol) and cesium carbonate (391 mg, 1.2 mmol) were successively added. Then the reaction solution was stirred at 120° C. overnight. After the reaction was completed, the reaction solution was concentrated under reduced pressure, diluted with water, and extracted with ethyl acetate. The combined organic phases were washed with saturated salt water, dried over anhydrous sodium sulfate, concentrated, and purified by column chromatography to obtain 65 mg of Compound C29-6.

1H NMR (400 MHz, CDCl₃) δ 10.93 (s, 1H), 8.88-8.87 (q, 1H), 8.46-8.40 (m, 2H), 7.63-7.60 (q, 1H), 7.57-7.53 (m, 1H), 3.49 (s, 4H), 1.87 (s, 3H), 1.84 (s, 3H).

(5) Synthesis of Compound C29

The Compound C29-6 (10 mg, 0.027 mmol) and Compound B (9 mg, 0.027 mmol) were dissolved in 1 ml of anhydrous dioxane, and then Pd₂(dba)₃ (3 mg, 0.0027 mmol), Xantphos (3.2 mg, 0.0054 mmol) and cesium carbonate (18 mg, 0.055 mmol) were successively added. Then the reaction solution was radiated by microwave at 150° C. for 1 h. After the reaction was completed, the reaction solution was concentrated under reduced pressure, diluted with water, extracted with ethyl acetate. The combined organic phases were washed with saturated salt water, dried over anhydrous sodium sulfate, concentrated, and purified by Prep-HPLC to obtain 2 mg of Compound C29.

1H NMR (400 MHz, CDCl₃) δ 12.00 (s, 1H), 8.71-8.70 (t, 2H), 8.30 (s, 3H), 8.14-8.12 (d, 1H, J=9.52 Hz), 8.08 (s, 1H), 7.51 (s, 1H), 7.49-7.46 (q, 1H), 6.69 (s, 1H), 3.87 (s, 3H), 3.11 (br, 2H), 3.02-2.92 (m, 6H), 2.71-2.65 (q, 2H), 2.48 (s, 3H), 2.45-2.43 (m, 2H), 2.17 (s, 3H), 2.11 (s, 3H).

Biological Activity Test

The experimental process was as follows:

The activity of the compounds prepared in Examples against EGFR(L858R/T790M/C797S) kinase was screened using the Kinase activity Assay method at ATP Km concentration, and staurosporine was used as a reference substance. The biological activity screening of the compounds will be determined repeatedly at 10 concentrations.

1. Sample to be tested

Each sample was prepared into a solution with a concentration of 10 mM.

2. Experimental method

a. Preparing basic buffer solution and quenching buffer solution for experimental kinase

20 mM Hepes (pH 7.5), 10 mM MgCl₂, 1 mM EGTA, 0.02% Brij35, 0.02 mg/ml BSA, 0.1 mM Na₃VO₄, 2 mM DTT, 1% DMSO.

b. Preparing compounds for experimental kinase

The test compound was dissolved in 100% dimethyl sulfoxide to a specific concentration. Integra Viaflo Assist was used to assist DMSO for (continuous) dilution.

c. Reaction steps:

Kinase was added to the newly prepared basic reaction buffer

Adding any desired cofactors to the substrate solution.

Adding EGFR (L858R/T790M/C797S) kinase into the substrate solution and gently mixing;

Acoustic technology (Echo 550; Nanoliter range) was used to feed the compounds in 100% of dimethyl sulfoxide into the kinase reaction mixture and incubated at room temperature for 20 minutes.

33P-ATP (specific activity 10 Ci/l) was added to the reaction mixture to start the reaction.

Incubated at room temperature for 2 hours

Radioactivity was detected by filter-binding method.

Kinase activity data was expressed as the percentage of remaining kinase activity in the test sample compared to the vehicle (dimethyl sulfoxide) reaction. Prism (GRAPHPAD software) was used to obtain IC50 value and curve-fitting.

The obtained inhibitory activity IC50 (nM) values of the samples against EGFR (L858R/T790M/C797S) kinase were shown in Table 1.

TABLE 1

| | L858R/T790M/ |
|---|---|
| Compound | C797S IC50 (nM) |
| Staurosporine | 0.527 |
| C1 | 12.4 |
| C2 | 16.6 |
| C3 | 130 |
| C4 | 107 |
| C5 | 321 |
| C6 | 178 |
| C7 | 22.3 |
| C8 | ND |
| C9 | 181 |
| C10 | 1850 |
| C11 | 17.6 |
| C12 | 74.7 |
| C13 | 16.6 |
| C14 | 13.7 |
| C19 | 243 |
| C20 | 69.6 |
| C22 | 0.3 |
| C23 | 20.5 |
| C24 | 19.5 |
| C25 | 803 |
| C27 | 204 |
| C28 | 803 |
| C29 | ND |

It can be seen from the above table that the synthesized compounds have good inhibitory ability against EGFR (L858R/T790M/C797S) kinase through in vitro biological activity screening compared to the reference substance Staurosporine, which can overcome the clinical drug resistance of non-small cell lung cancer patients induced by the existing third generation selective EGFRT790M small molecule inhibitors, and the compounds are expected to be further developed into drugs for regulating EGFR (L858R/T790M/C797S) kinase activity or treating EGFR (L858R/T790M/C797S) related diseases.

The above description is only examples of the present invention, and does not limit the patent scope of the present invention. Any equivalent modifications made by using the contents of the present specification or directly or indirectly applied to other related technical fields are included in the scope of the present invention.

1. A compound of formula I or a pharmaceutically acceptable salt, solvate or prodrug thereof, 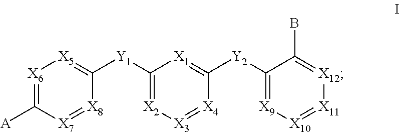
wherein,
X₁ is selected from N or CR₁;
X₂ is selected from N or CR₂;
X₃ is selected from N or CR₃;
X₄ is selected from N or CR₄;
X₅ is selected from N or CR₅;
X₆ is selected from N or CR₆;
X₇ is selected from N or CR₇;
X₈ is selected from N or CR₈;
X₉ is selected from N or CR₉;
X₁₀ is selected from N or CR₁₀;
X₁₁ is selected from N or CR₁₁;
X₁₂ is selected from N or CR₁₂;
Y₁ and Y₂ are each independently selected from the divalent group consisting of —O—, —S—, —S(O)—, —S(O)₂—, 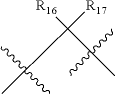
and —NR₁₈—;
A is selected from the group consisting of 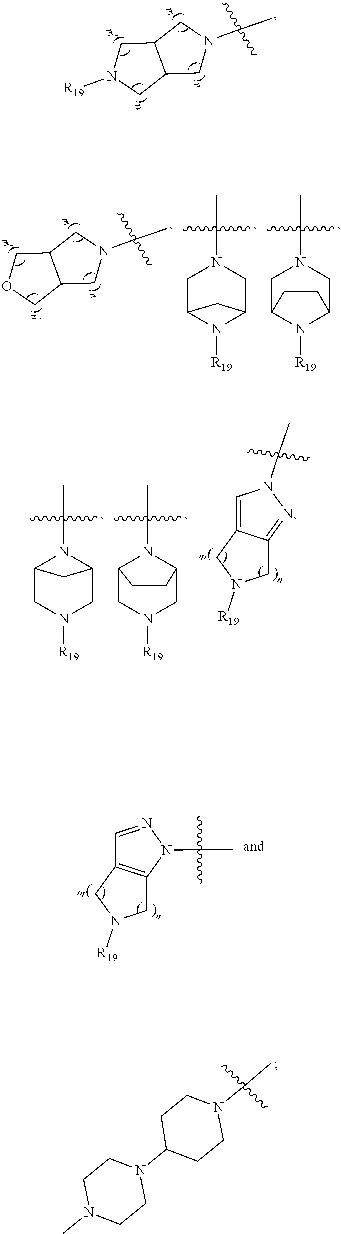
or A and X₇ or X₆ form a substituted 5-7 membered ring;
B is selected from the group consisting of 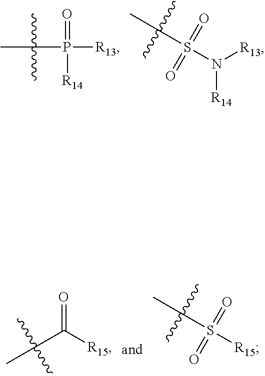
R₁, R₂, R₃, R₄, R₅, R₆, R₇, R₈, R₉, R₁₀, R₁₁ and R₁₂ are each independently selected from the substituted or unsubstituted group consisting of H, halogen, CN, NH₂, ester, carbamido, carbamate, amido, C₁₋₆ alkyl, C₁₋₆ alkoxy, C₃₋₆ cycloalkyl, C₃₋₆ cycloalkoxy, sulfonamido, amino, 3-10 membered heterocyclyl, C₆-C₁₀ aryl, 5-14 membered heteroaryl;
or R₃ and X₂ or X₄ form a substituted or unsubstituted 5-7 membered ring containing 0-3 heteroatoms selected from O, S, or N;
or R₁₁ and X₁₀ or X₁₂ form a substituted or unsubstituted 5-7 membered ring containing 0-3 heteroatoms selected from O, S, or N;
or R₁₀ and X₉ or X₁₁ form a substituted or unsubstituted 5-7 membered ring containing 0-3 heteroatoms selected from O, S, or N;
or R₅ and X₆ form a substituted or unsubstituted 5-7 membered ring containing 0-3 heteroatoms selected from O, S, or N;
or R₇ and X₈ form a substituted or unsubstituted 5-7 membered ring containing 0-3 heteroatoms selected from O, S, or N;
R₁₃, R₁₄ and R₁₅ are each independently selected from the substituted or unsubstituted group consisting of H, C₁₋₆ alkyl, C₁₋₆ alkoxy, C₃₋₈ cycloalkyl, C₃₋₈ cycloalkoxy, C₆-C₁₀ aryl, 5-14 membered heteroaryl;
or R₁₃ and R₁₄ together with the P or N atoms to which they are attached form a substituted or unsubstituted 4-8 membered heterocyclyl;
R₁₆, R₁₇ and R₁₈ are each independently selected from the substituted or unsubstituted group consisting of H, halogen, C₁₋₆ alkyl, C₁₋₆ alkoxy, C₃₋₈ cycloalkyl, C₃₋₈ cycloalkoxy, C₆-C₁₀ aryl, and 5-14 membered heteroaryl;
or R₁₆ and R₁₇ together with the C atoms to which they are attached form a substituted or unsubstituted C₄₋₈ cycloalkyl or 4-8 membered heterocyclyl;
R₁₉ is selected from the substituted or unsubstituted group consisting of H, C₁₋₆ alkyl, C₁₋₆ alkoxy, C₃₋₈ cycloalkyl, C₃₋₈ cycloalkoxy, C₆-C₁₀ aryl, 5-14 membered heteroaryl, C₁₋₆ alkoxycarbonyl, C₁₋₆ alkylcarbonyl, and C₁₋₆ alkyl-S(═O)₂—;
m, n, m′ and n′ are each independently 0, 1, 2, or 3;
with the proviso that
when A is 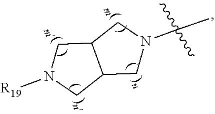
X₁ is CR₁ or X₂ is CR₂,
or X₅ is CR₅, and R₅ is selected from the substituted or unsubstituted group consisting of halogen, CN, NH₂, ester, carbamido, carbamate, amido, C₁₋₆ alkyl, C₁₋₆ alkoxy, C₃₋₆ cycloalkyl, C₃₋₆cycloalkoxy, C₆-C₁₀ aryl, and 5-14 membered heteroaryl;
or X₆ is CR₆, and R₆ is selected from the substituted or unsubstituted group consisting of halogen, CN, NH₂, ester, carbamido, carbamate, amido, C₁₋₆ alkyl, C₁₋₆ alkoxy, C₃₋₆ cycloalkyl, C₃₋₆ cycloalkoxy, C₆-C₁₀ aryl, and 5-14 membered heteroaryl;
or X₈ is selected from CR₈, and R₈ is selected from the substituted or unsubstituted group consisting of halogen, CN, NH₂, ester, carbamido, carbamate, amido, C₁₋₆ alkyl, C₁₋₆ alkoxy, C₃₋₆ cycloalkyl, C₃₋₆ cycloalkoxy, C₆-C₁₀ aryl, and 5-14 membered heteroaryl;
or with the proviso that
when A is 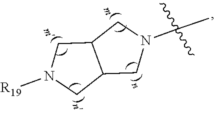
and when both X₁ and X₂ are N,
R₃ and X₄ form a substituted or unsubstituted 5-7 membered ring containing 0-3 heteroatoms selected from O, S, or N;
or R₁₁ and X₁₀ or X₁₂ form a substituted or unsubstituted 5-7 membered ring containing 0-3 heteroatoms selected from O, S, or N;
or R₁₀ and X₉ or X₁₁ form a substituted or unsubstituted 5-7 membered ring containing 0-3 heteroatoms selected from O, S, or N;
or R₅ and X₆ form a substituted or unsubstituted 5-7 membered ring containing 0-3 heteroatoms selected from O, S, or N;
or R₇ and X₈ form a substituted or unsubstituted 5-7 membered ring containing 0-3 heteroatoms selected from O, S, or N;
or R₁₃ and R₁₄ together with the P atom to which they are attached form a substituted or unsubstituted 4-8 membered heterocyclyl;
or B is selected from the group consisting of 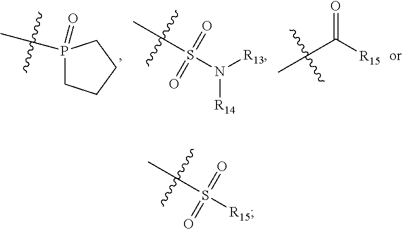
or with the proviso that
when A is 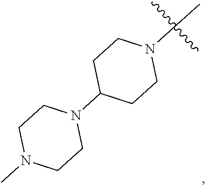 [SMILES: *N1CCC(N2CCN(C)CC2)CC1]
and
X₁ and X₂ are not N at the same time;
R₃ and X₂ or X₄ form a substituted or unsubstituted 5-7 membered ring containing 0-3 heteroatoms selected from O, S, or N;
or R₁₁ and X₁₀ or X₁₂ form a substituted or unsubstituted 5-7 membered ring containing 0-3 heteroatoms selected from O, S, or N;
or R₁₀ and X₁₀ or X₁₁ form a substituted or unsubstituted 5-7 membered ring containing 0-3 heteroatoms selected from O, S, or N;
or R₅ and X₆ form a substituted or unsubstituted 5-7 membered ring containing 0-3 heteroatoms selected from O, S, or N;
or R₇ and X₈ form a substituted or unsubstituted 5-7 membered ring containing 0-3 heteroatoms selected from O, S, or N;
or R₁₃ and R₁₄ together with the P atom to which they are attached form a substituted or unsubstituted 4-8 membered heterocyclyl;
or B is selected from the group consisting of 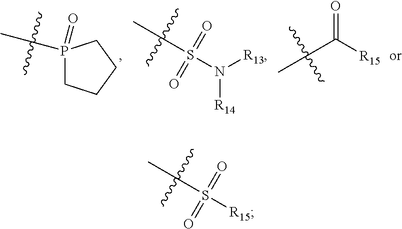
wherein, the term “substituted” means being substituted by one or more groups selected from the group consisting of deuterium, halogen, CN, OH, NH₂, ester, carbamido, carbamate, amido, C₁₋₆ alkyl, C₁₋₆ alkoxy, C₃₋₆ cycloalkyl, C₃₋₆ cycloalkoxy, C₂₋₆ alkenyl, C₂₋₆ alkynyl, C₆-C₁₀ aryl, 5-14 membered heteroaryl, and 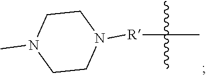
R′ is selected from the group consisting of C₁₋₆ alkylene, C₁₋₆ alkylene-CO—, and —CO—C₁₋₆ alkylene.

2. The compound of formula I of claim 1, or the pharmaceutically acceptable salt, solvate or prodrug thereof, wherein, R₈ is selected from the substituted or unsubstituted group consisting of H, halogen, CN, NH₂, ester, carbamido, carbamate, amido, C₁₋₆ alkyl and C₁₋₆ alkoxy;
wherein the term “substituted” means being substituted by one or more substituents selected from the group consisting of deuterium, halogen, CN, OH, NH₂, C₁₋₆ alkyl and C₁₋₆ alkoxy.

3. The compound of formula I of claim 1, or the pharmaceutically acceptable salt, solvate or prodrug thereof, wherein, A and X₇ or X₆ form a substituted 5-7 membered ring,
wherein, the term “substituted” means that H on the 5-7 membered ring is substituted by one or more groups selected from the group consisting of halogen, CN, OH, NH₂, ester, carbamido, carbamate, amido, C₁₋₆ alkyl, C₁₋₆ alkoxy, C₃₋₆ cycloalkyl, C₃₋₆ cycloalkoxy, C₂₋₆ alkenyl C₂₋₆ alkynyl, C₆-C₁₀ aryl, 5-14 membered heteroaryl, and 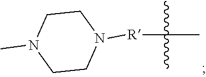
R′ is selected from the group consisting of C₁₋₆ alkylene, C₁₋₆alkylene-CO—, and —CO—C₁₋₆ alkylene.

4. The compound of formula I of claim 1, or the pharmaceutically acceptable salt solvate or prodrug thereof; wherein,
when A is 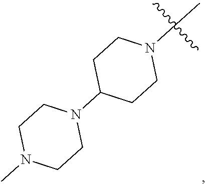 [SMILES: *N1CCC(N2CCN(C)CC2)CC1]
X₁ is CR₁ or X₂ is CR₂;
or R₃ and X₂ or X₄ form a substituted or unsubstituted 5-7 membered ring containing 0-3 heteroatoms selected from O, S, or N;
or R₁₁ and X₁₀ or X₁₂ form a substituted or unsubstituted 5-7 membered ring containing 0-3 heteroatoms selected from O, S, or N; R₁₀ and X₉ or X₁₁ form a substituted or unsubstituted 5-7 membered ring containing 0-3 heteroatoms selected from O, S, or N;
wherein, the term “substituted” means being substituted by one or more groups selected from the group consisting of halogen, CN, OH, NH₂, ester, carbamido, carbamate, amido, C₁₋₆ alkyl, C₁₋₆ alkoxy, C₃₋₆ cycloalkyl, C₃₋₆ cycloalkoxy, C₂₋₆ alkenyl, C₂₋₆ alkynyl, C₆-C₁₀ aryl, 5-14 membered heteroaryl, and 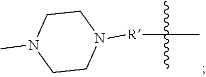
R′ is selected from the group consisting of C₁₋₆ alkylene, C₁₋₆ alkylene-CO—, and —CO—C₁₋₆ alkylene.

5. The compound of formula I of claim 1, or the pharmaceutically acceptable salt, solvate or prodrug thereof;
wherein the compound has the structure represented by formula II, formula II′, formula III, formula IV or formula V, 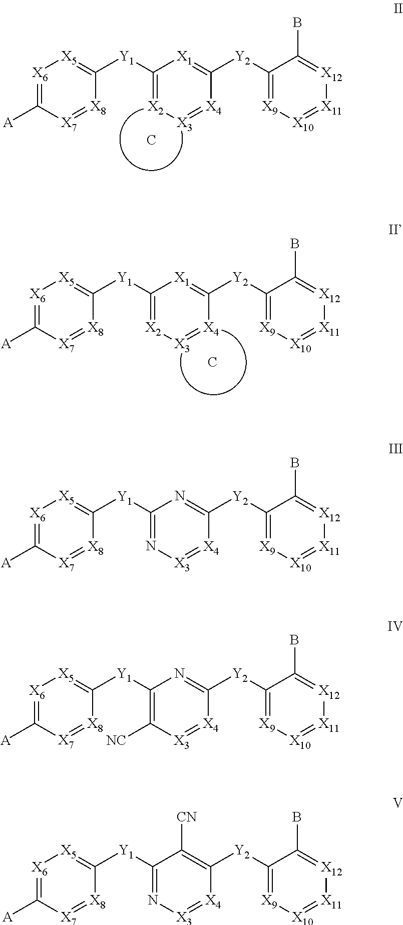
wherein,
C ring is a substituted or unsubstituted 5-7 membered ring;
X₁, X₂, X₃, X₄, X₅, X₆, X₇, X₈, X₉, X₁₀, X₁₁, X₁₂, Y₁, Y₂, A and B are as defined in claim 1, with the proviso that
in formula III, when A is 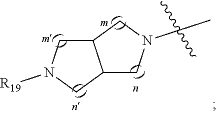
R₁₁ and X₁₀ or X₁₂ form a substituted or unsubstituted 5-7 membered ring containing 0-3 heteroatoms selected from O, S, or N; or X₃ and X₄ are each independently N;
or X₃ is CR₃, X₄ is CR₄, wherein R₃ and R₄ are each independently selected from the group consisting of halogen, CN, NH₂, ester, carbamido, carbamate, amido, C₁₋₆ alkyl, C₁₋₆ alkoxy, C₃₋₆ cycloalkyl, C₃₋₆ cycloalkoxy, sulfonamido, amino, 3-10 membered heterocyclyl, C₆-C₁₀ aryl, and 5-14 membered heteroaryl;
or R₃ and R₄ form a substituted or unsubstituted 5-7 membered ring containing 0-3 heteroatoms selected from O, S, or N;
wherein, the term “substituted” means being substituted by one or more groups selected from the group consisting of halogen, CN, OH, NH₂, ester, carbamido, carbamate, amido, C₁₋₆ alkyl, C₁₋₆ alkoxy, C₃₋₆ cycloalkyl, C₃₋₆ cycloalkoxy, C₂₋₆ alkenyl, C₂₋₆ alkynyl, C₆-C₁₀ aryl, and 5-14 membered heteroaryl;
wherein, R₁₉, m, n, m′, n′ and R₁₁ are as defined in claim 1.

6. The compound of formula I of claim 1, or the pharmaceutically acceptable salt solvate or prodrug thereof;
wherein 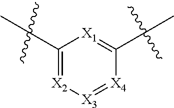
moiety is selected from the group consisting of: 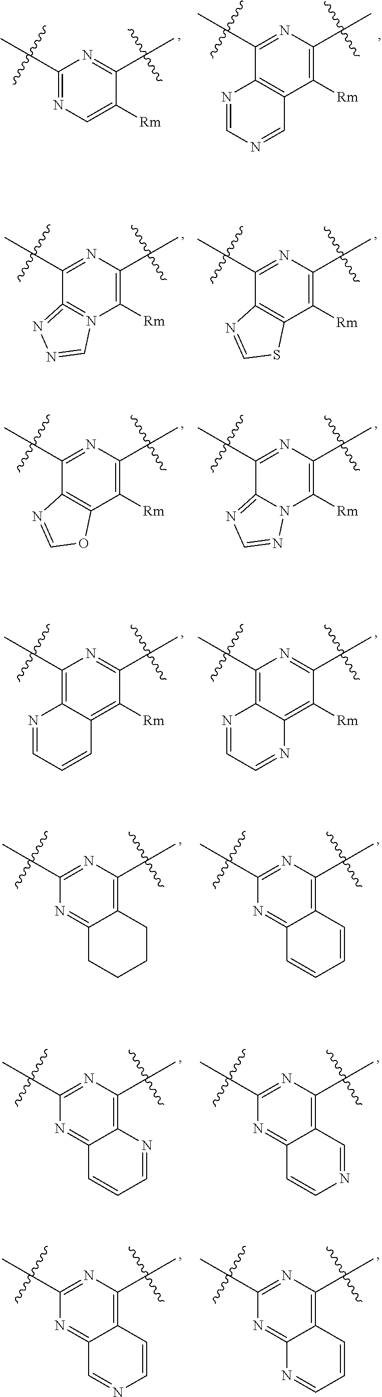 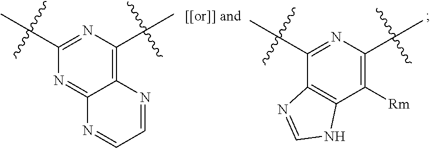
wherein Rm is a halogen.

7. The compound of formula I of claim 1, or the pharmaceutically acceptable salt, solvate or prodrug thereof;
wherein 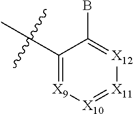
moiety is selected from the group consisting of:

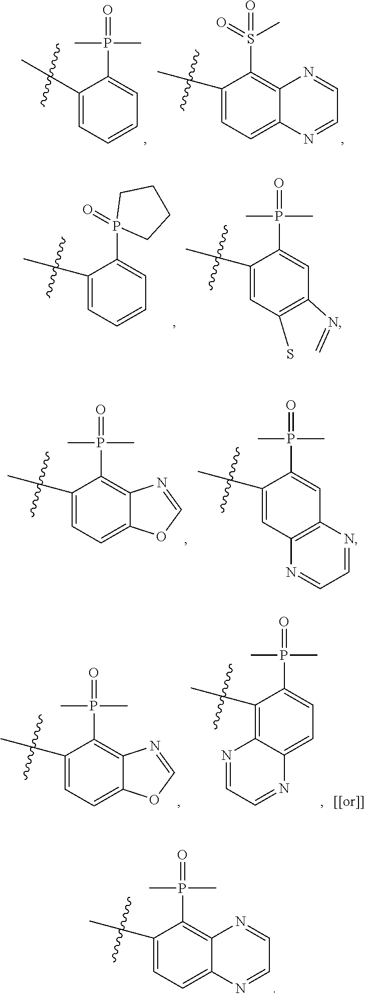

8. The compound of formula I of claim 1, or the pharmaceutically acceptable salt, solvate or prodrug thereof;
wherein the compound has the structure represented by formula VI, formula VII, formula VIII or formula IX, 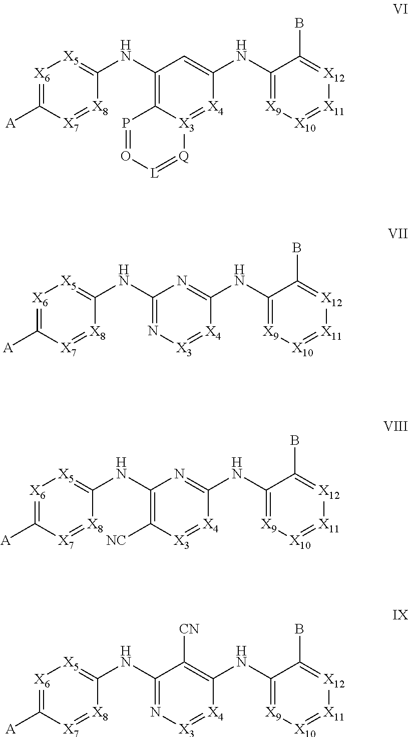
wherein O, P, Q and L are each independently selected from or CR₁;
with the proviso that in VII, when A is 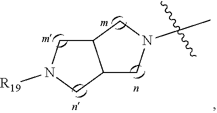
R₁₁ and X₁₀ or X₁₂ form a substituted or unsubstituted 5-7 membered ring containing 0-3 heteroatoms selected from O, S, or N;
or R₃ and X₂ or X₄ form a substituted or unsubstituted 5-7 membered ring containing 0-3 heteroatoms selected from O, S, or N;
wherein the term “substituted” means being substituted by one or more groups selected from the group consisting of halogen, CN, OH, NH₂, ester, carbamido, carbamate, amido, C₁₋₆ alkyl, C₁₋₆ alkoxy, C₃₋₆ cycloalkyl, C₃₋₆ cycloalkoxy, C₂₋₆ alkenyl, C₂₋₆ alkynyl, C₆-C₁₀ aryl, and 5-14 membered heteroaryl;
wherein X₁, X₃, X₄, X₅, X₆, X₇, X₈, X₉, X₁₀, X₁₁, X₁₂, R₁₁, R₁₉, A, B, m, n, m′ and n′ are as defined in claim 1.

9. The compound of formula I of claim 1, or the pharmaceutically acceptable salt, solvate or prodrug thereof;
wherein R₈ is deuterated C₁₋₆ alkoxy, deuterated C₁₋₆ alkyl, deuterated C₁₋₆ haloalkoxy, or deuterated C₁₋₆ haloalkyl.

10. The compound of formula I of claim 1, or the pharmaceutically acceptable salt, solvate or prodrug thereof;
Wherein R₈ is selected from the group consisting of —O—CDF₂, —O—CD₃-, —O—CD₂F, —O—CF₃, —CD₃, —CDF₂ and —CD₂F.

11. The compound of formula I of claim 1, or the pharmaceutically acceptable salt, solvate or prodrug thereof, wherein, the compound is selected from the group consisting of

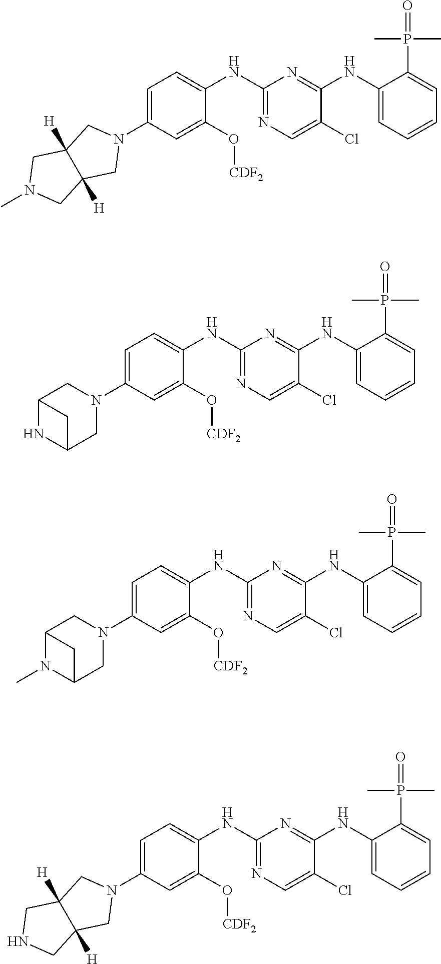

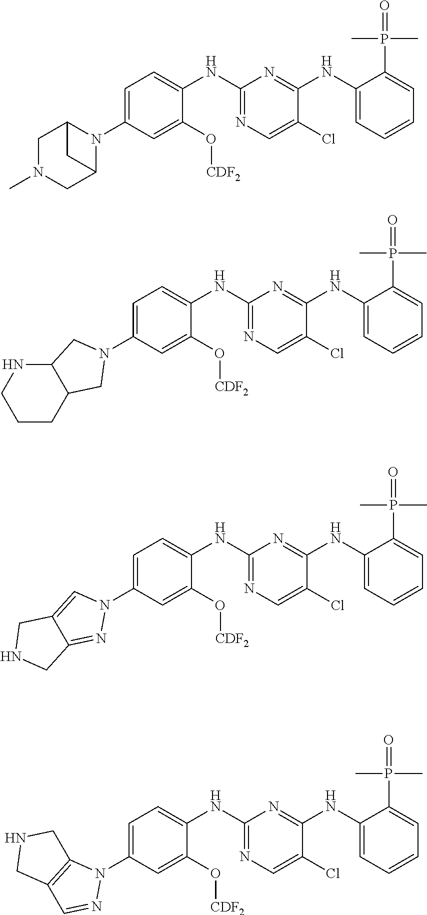

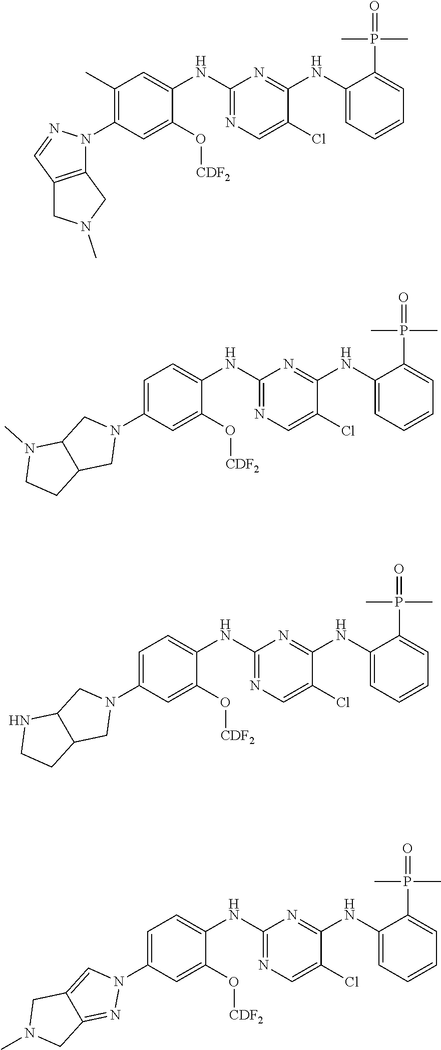

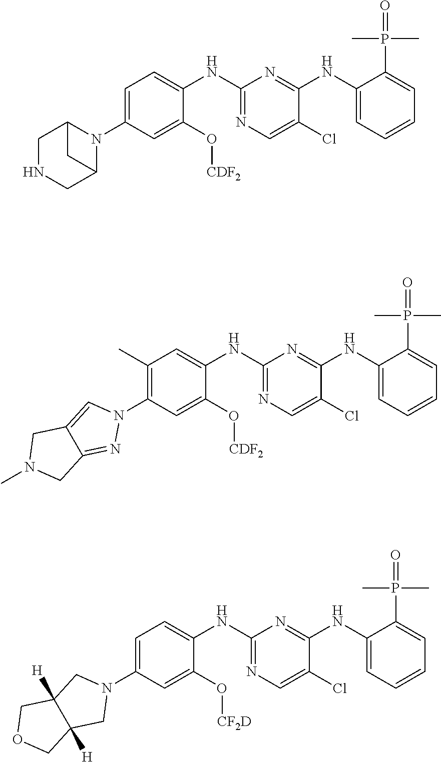

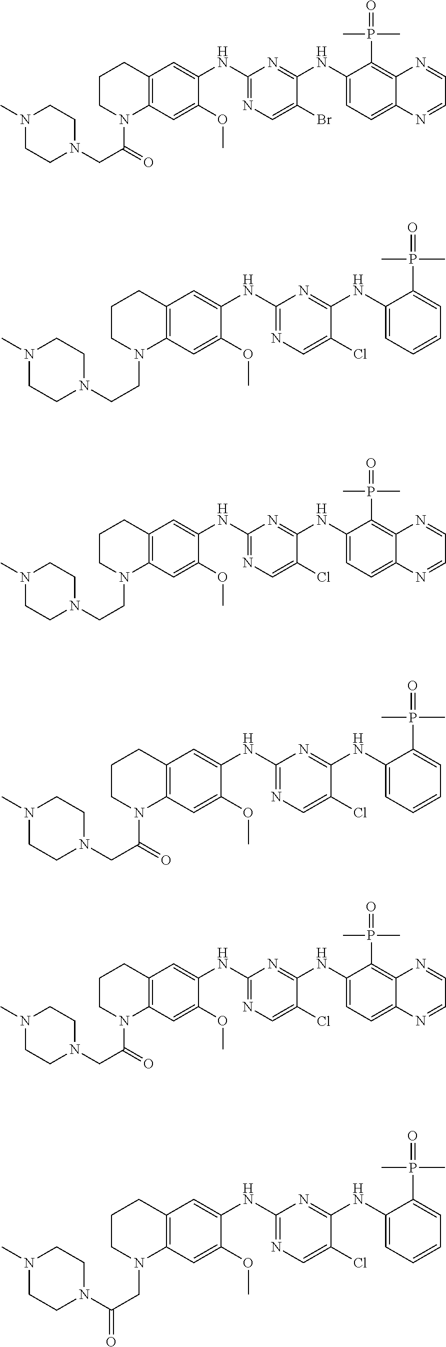

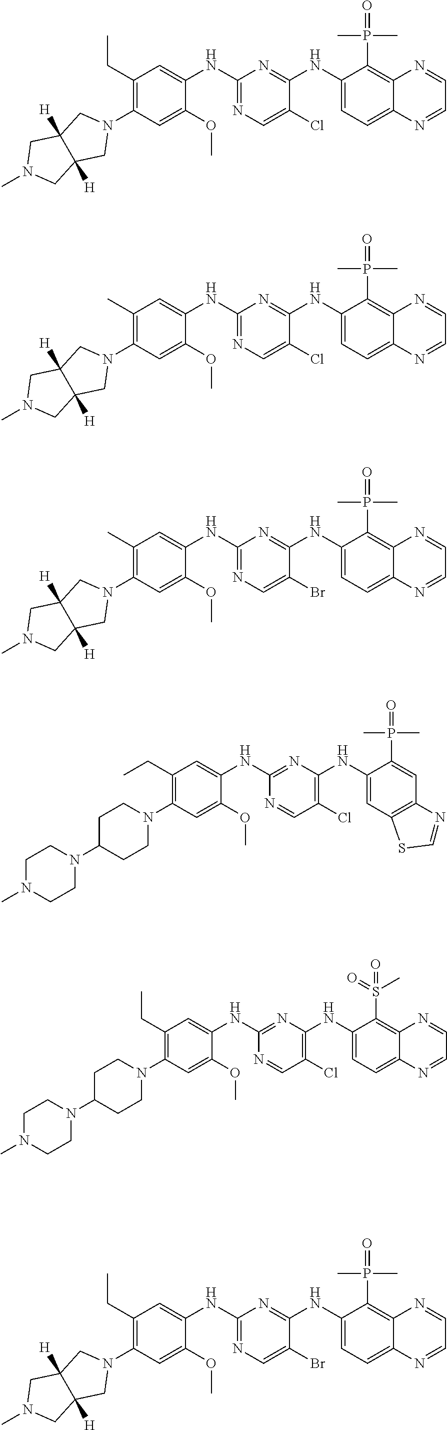 [SMILES: CCc1cc(Nc2ncc(Cl)c(Nc3ccc4nccnc4c3P(C)(C)=O)n2)c(OC)cc1N1C[C@H]2CN(C)C[C@H]2C1 ; COc1cc(N2C[C@H]3CN(C)C[C@H]3C2)c(C)cc1Nc1ncc(Cl)c(Nc2ccc3nccnc3c2P(C)(C)=O)n1 ; COc1cc(N2C[C@H]3CN(C)C[C@H]3C2)c(C)cc1Nc1ncc(Br)c(Nc2ccc3nccnc3c2P(C)(C)=O)n1 ; CCc1cc(Nc2ncc(Cl)c(Nc3cc4scnc4cc3P(C)(C)=O)n2)c(OC)cc1N1CCC(N2CCN(C)CC2)CC1 ; CCc1cc(Nc2ncc(Cl)c(Nc3ccc4nccnc4c3S(C)(=O)=O)n2)c(OC)cc1N1CCC(N2CCN(C)CC2)CC1 ; CCc1cc(Nc2ncc(Br)c(Nc3ccc4nccnc4c3P(C)(C)=O)n2)c(OC)cc1N1C[C@H]2CN(C)C[C@H]2C1]

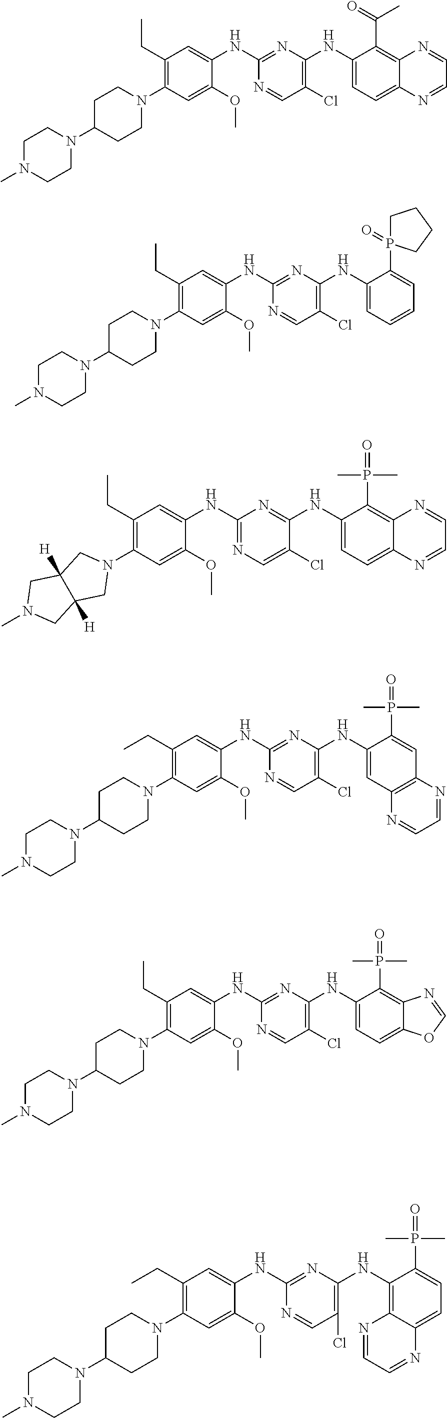

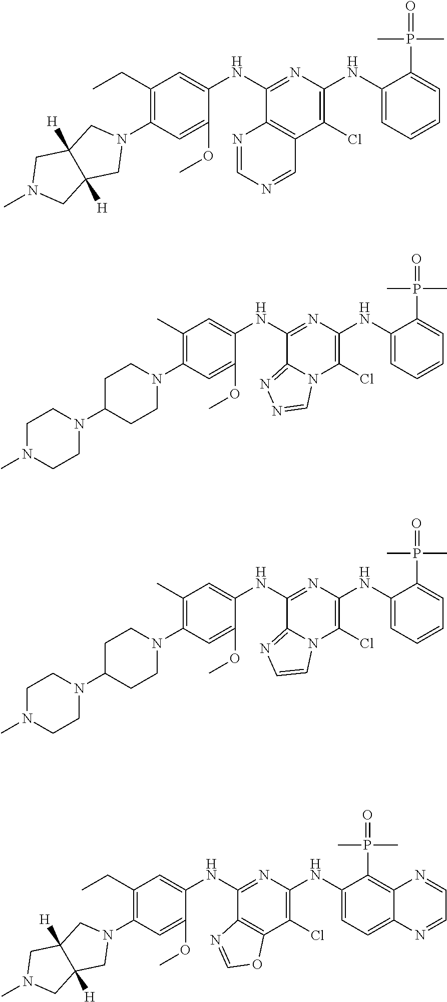

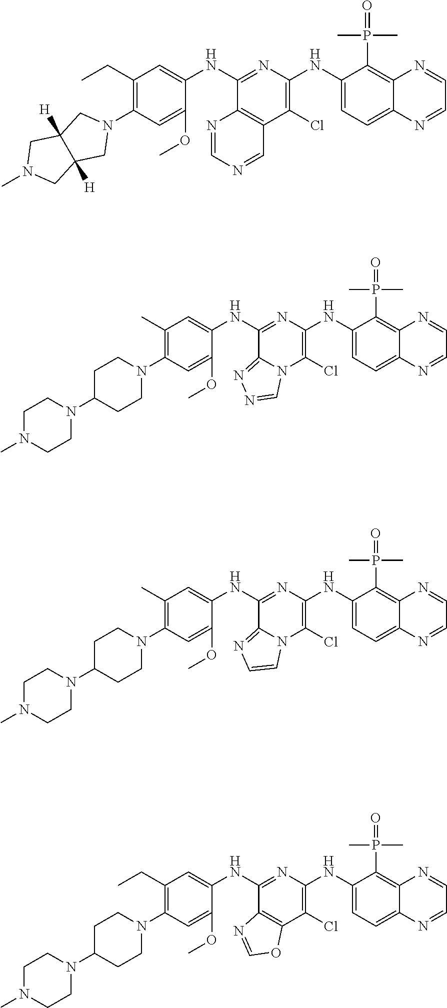 [SMILES: CCc1cc(Nc2nc(Nc3ccc4nccnc4c3P(C)(C)=O)c(Cl)c3cncnc23)c(OC)cc1N1C[C@H]2CN(C)C[C@H]2C1 ; COc1cc(N2CCC(N3CCN(C)CC3)CC2)c(C)cc1Nc1nc(Nc2ccc3nccnc3c2P(C)(C)=O)c(Cl)n2cnnc12 ; COc1cc(N2CCC(N3CCN(C)CC3)CC2)c(C)cc1Nc1nc(Nc2ccc3nccnc3c2P(C)(C)=O)c(Cl)n2ccnc12 ; CCc1cc(Nc2nc(Nc3ccc4nccnc4c3P(C)(C)=O)c(Cl)c3ocnc23)c(OC)cc1N1CCC(N2CCN(C)CC2)CC1]

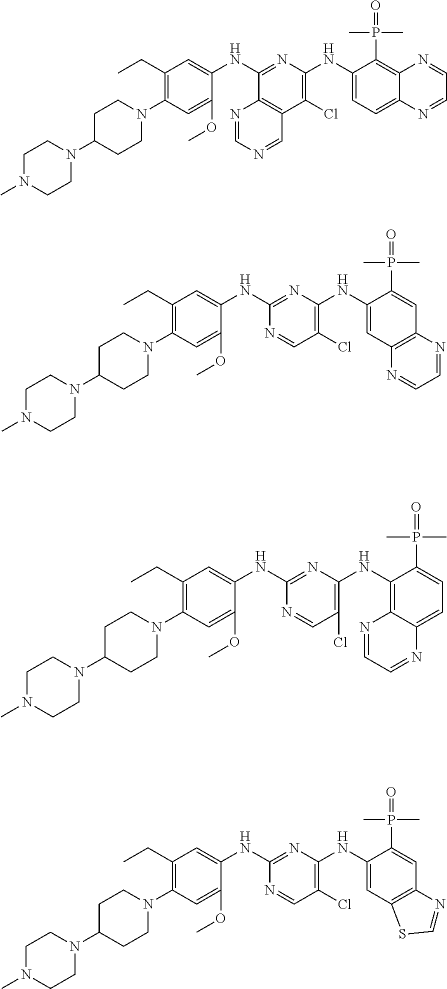

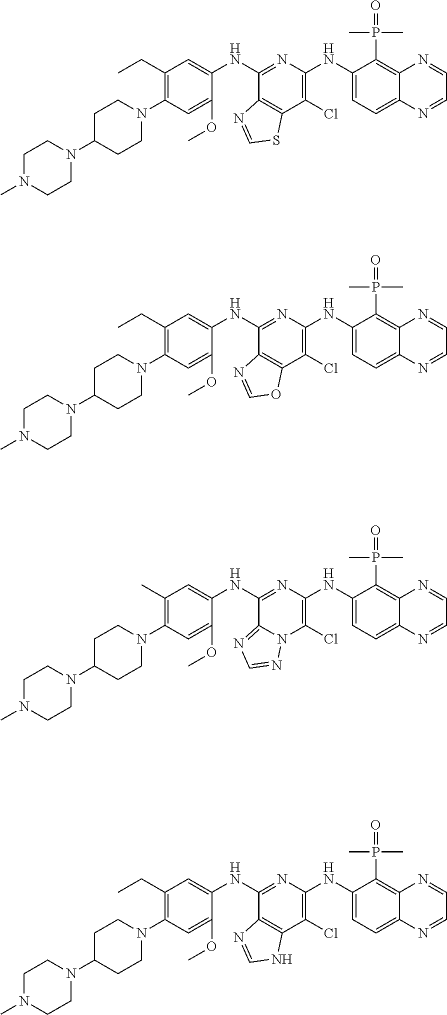 [SMILES: CCc1cc(Nc2nc(Nc3ccc4nccnc4c3P(C)(C)=O)c(Cl)c3scnc23)c(OC)cc1N1CCC(N2CCN(C)CC2)CC1 ; CCc1cc(Nc2nc(Nc3ccc4nccnc4c3P(C)(C)=O)c(Cl)c3ocnc23)c(OC)cc1N1CCC(N2CCN(C)CC2)CC1 ; COc1cc(N2CCC(N3CCN(C)CC3)CC2)c(C)cc1Nc1nc(Nc2ccc3nccnc3c2P(C)(C)=O)c(Cl)n2ncnc12 ; CCc1cc(Nc2nc(Nc3ccc4nccnc4c3P(C)(C)=O)c(Cl)c3[nH]cnc23)c(OC)cc1N1CCC(N2CCN(C)CC2)CC1]

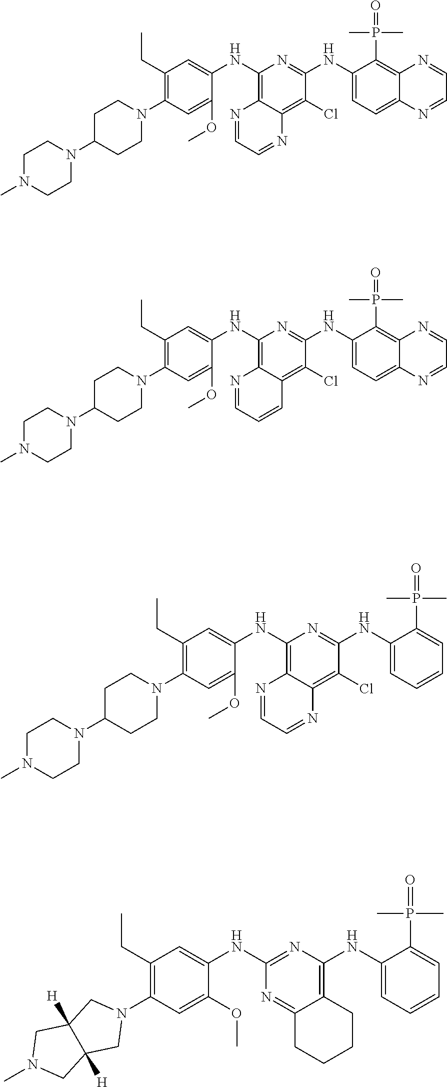

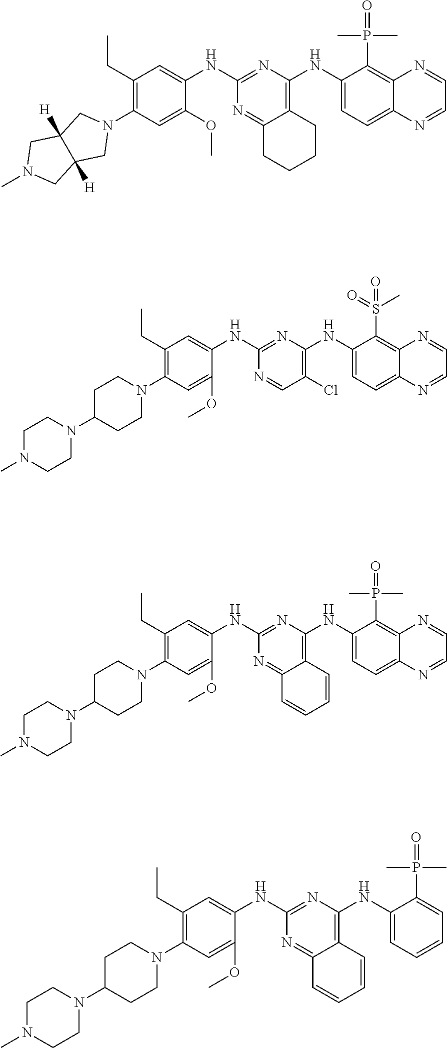

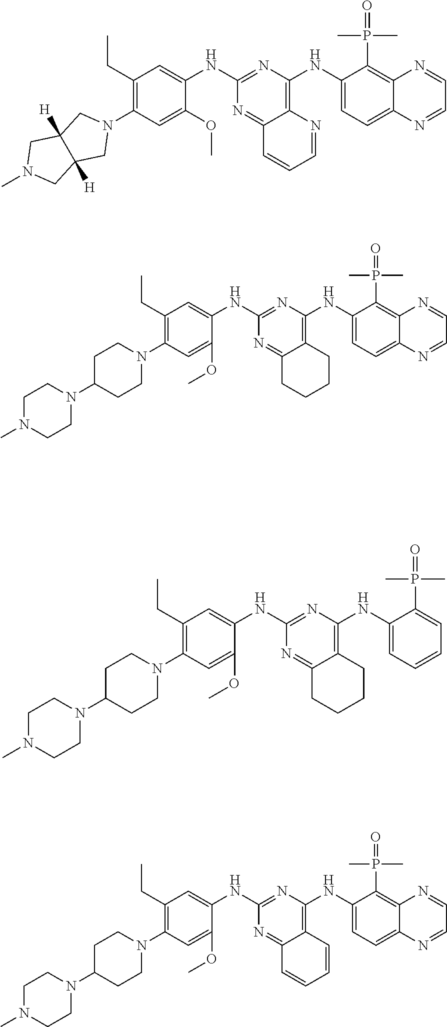

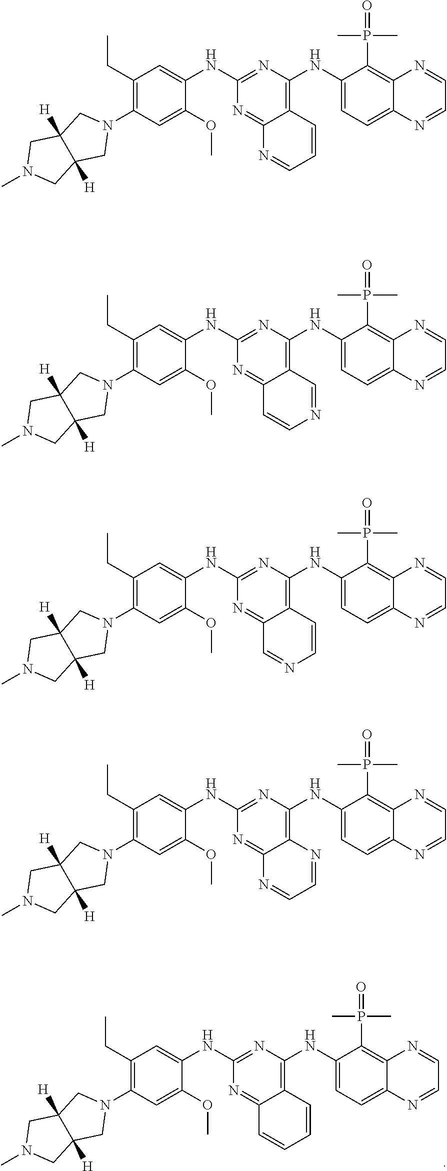 [SMILES: CCc1cc(Nc2nc(Nc3ccc4nccnc4c3P(C)(C)=O)c3cccnc3n2)c(OC)cc1N1C[C@H]2CN(C)C[C@H]2C1 ; CCc1cc(Nc2nc(Nc3ccc4nccnc4c3P(C)(C)=O)c3cnccc3n2)c(OC)cc1N1C[C@H]2CN(C)C[C@H]2C1 ; CCc1cc(Nc2nc(Nc3ccc4nccnc4c3P(C)(C)=O)c3ccncc3n2)c(OC)cc1N1C[C@H]2CN(C)C[C@H]2C1 ; CCc1cc(Nc2nc(Nc3ccc4nccnc4c3P(C)(C)=O)c3nccnc3n2)c(OC)cc1N1C[C@H]2CN(C)C[C@H]2C1 ; CCc1cc(Nc2nc(Nc3ccc4nccnc4c3P(C)(C)=O)c3ccccc3n2)c(OC)cc1N1C[C@H]2CN(C)C[C@H]2C1]

12. A pharmaceutical composition comprising the compound of claim 1, or the pharmaceutically acceptable salt, solvate or prodrug thereof; and a pharmaceutically acceptable carrier.

13. A method of treating cancer comprising administering a therapeutically effective amount of the compound of claim 1, or the pharmaceutically acceptable salt, solvate or prodrug thereof to a subject in need thereof.

14. The method of claim 13, wherein the cancer is lung cancer caused by EGFR C797S mutation.

15. The method of claim 13, wherein the cancer is selected from the group consisting of non-small cell lung cancer, small cell lung cancer, lung adenocarcinoma, lung squamous cell carcinoma, pancreatic cancer, breast cancer, prostate cancer, liver cancer, skin cancer, epithelial cell carcinoma, gastrointestinal stromal tumor, leukemia, histiocytic lymphoma, nasopharyngeal carcinoma, head and neck cancer, colon cancer, rectal cancer, glioma and a combination thereof.

16. The method of claim 13, wherein the cancer is small cell lung cancer or non-small cell lung cancer.

17. The method of claim 13, wherein the cancer is lung cancer caused by EGFR L858R/T790M/C797S mutations.

18. The compound of claim 1, with the proviso that
when A is, 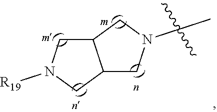
X₁ is CR₁ and X₂ is CR₂,
or X₅ is CR₅, and R₅ is selected from the substituted or unsubstituted group consisting of halogen, CN, NH₂, ester, carbamido, carbamate, amido, C₁₋₆ alkyl, C₁₋₆ alkoxy, C₃₋₆ cycloalkyl, C₃₋₆cycloalkoxy, C₆-C₁₀ aryl, and 5-14 membered heteroaryl;
or X₆ is CR₆, and R₆ is selected from the substituted or unsubstituted group consisting of halogen, CN, NH₂, ester, carbamido, carbamate, amido, C₁₋₆ alkyl, C₁₋₆ alkoxy, C₃₋₆ cycloalkyl, C₃₋₆ cycloalkoxy, C₆-C₁₀ aryl, and 5-14 membered heteroaryl;
or X₈ is selected from CR₈, and R₈ is selected from the substituted or unsubstituted group consisting of halogen, CN, NH₂, ester, carbamido, carbamate, amido, C₁₋₆ alkyl, C₁₋₆ alkoxy, C₃₋₆ cycloalkyl, C₃₋₆ cycloalkoxy, C₆-C₁₀ aryl, and 5-14 membered heteroaryl.

19. The compound of formula I of claim 1, or the pharmaceutically acceptable salt, solvate or prodrug thereof; wherein
when A is 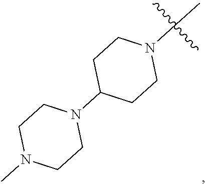 [SMILES: *N1CCC(N2CCN(C)CC2)CC1]
X₁ is CR₁ and X₂ is CR₂;
or R₃ and X₂ or X₄ form a substituted or unsubstituted 5-7 membered ring containing 0-3 heteroatoms selected from O, S, or N;
or R₁₁ and X₁₀ or X₁₂ form a substituted or unsubstituted 5-7 membered ring containing 0-3 heteroatoms selected from O, S, or N; R₁₀ and X₉ or X₁₁ form a substituted or unsubstituted 5-7 membered ring containing 0-3 heteroatoms selected from O, S, or N;
wherein, the term “substituted” means being substituted by one or more groups selected from the group consisting of halogen, CN, OH, NH₂, ester, carbamido, carbamate, amido, C₁₋₆ alkyl, C₁₋₆ alkoxy, C₃₋₆ cycloalkyl, C₃₋₆ cycloalkoxy, C₂₋₆ alkenyl, C₂₋₆ alkynyl, C₆-C₁₀ aryl, 5-14 membered heteroaryl, and 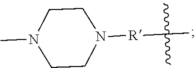
R′ is selected from the group consisting of C₁₋₆ alkylene, C₁₋₆ alkylene-CO—, and —CO—C₁₋₆ alkylene.
